(19) United States
(12) Patent Application Publication
(10) Pub. No.: US 2008/0027226 A1
(43) Pub. Date: Jan. 31, 2008

(54) Electroluminescent Device

(76) Inventors: Jonathan Rogers, White Plains, NY (US); Francois Maike, Geispitzen (FR); Norihisa Dan, Kyoto (JP)

Correspondence Address: CIBA SPECIALTY CHEMICALS CORPORATION;PATENT DEPARTMENT, 540 WHITE PLAINS RD, P O BOX 2005, TARRYTOWN, NY, 10591-9005, US

(21) Appl. No.: 10/581,398

(22) PCT Filed: Nov. 26, 2004
(86) PCT No.: PCT/EP04/53111

§ 371 Date: Mar. 22, 2007

(30) Foreign Application Priority Data
Dec. 5, 2003 (EP) 03104562.8
Sep. 6, 2004 (EP) 04104279.7

(51) Int. Cl. C07D 249/16; C07D 401/04; C07D 493/00

(52) U.S. Cl. 546/199; 548/257; 548/260

(57) ABSTRACT

Disclosed are electroluminescent devices that comprise organic layers that contain certain 2H-benzotriazole compounds. The 2H-benzotriazole compounds are suitable components of blue-emitting, durable, organo-electroluminescent layers. The electroluminescent devices may be employed for full color display panels in, for example, mobile phones, televisions and personal computer screens.

The present invention relates to organo-electroluminescent (EL) devices, in particular EL devices that comprise durable, blue-emitting organo-electroluminescent layers. The organo-electroluminescent layers comprise certain 2H-benzotriazoles.

Progress has been made towards developing organic-based electroluminescent devices suitable for full color displays. Generally, an EL device is comprised of a light-emitting layer or layers and a pair of facing electrodes sandwiching the light-emitting layer(s). Application of an electric field between the electrodes results in the injection of electrons and holes to the system, resulting in the release of energy as light.

However, organo EL devices have not been developed that have suitable stability under continuous operation. In particular, there remains a need for blue-emitting, stable organo EL devices.

U.S. Pat. No. 5,104,740 teaches an electroluminescent element that comprises a fluorescent layer containing a coumarinic or azacoumarinic derivative and a hole transport layer, both made of organic compounds and laminated on top of the other. Certain of the coumarinic compounds disclosed have 2H-benzotriazole substitutents.

U.S. Pat. No. 6,280,859 discloses certain polyaromatic organic compounds for use as a light-emitting material in organo-electroluminescent devices. A 2H-benzotriazole moiety is listed among a long list of possible divalent aromatic linking groups.

U.S. Pat. No. 5,116,708 is aimed at a hole transport material for EL devices.

U.S. Pat. No. 5,518,824 teaches an EL device comprising one or more organic layers, wherein at least one of the layers is obtained by thermal or radiation-induce crosslinking. Certain benzotriazoles are disclosed as suitable charge transport compounds.

U.S. Pat. No. 4,533,612 discloses electrophotographic recording materials that comprise certain 2H-benzotriazoles as charge carrier-transporting compounds.

JP58009151 discloses the use of certain polyaromatic benzotriazole systems in a charge transport layer of an electrophotographic photoreceptor.

U.S. Pat. No. 5,629,389 discloses an electroluminescent device having a layer that comprises 2-(2H-benzotriaol-2-yl)-4,6-bis(1-methyl-1-phenylethyl)phenol.

EP764712 discloses ortho hydroxyphenyl-2H-benzotriazoles as stabilizers in EL devices. Tsutsui, et al., in Synthetic Metals, 1997 (85) 1201-1204, discloses 2-(2-hydroxy-5-methylphenyl)-2H-benzotriazole as a fluorescence quencher in an electron transport layer.

U.S. Pat. Nos. 2,784,183, 2,713,056, 2,784,197, 3,288,786, 3,341,530, 5,006,662, GB-A-1150408, DE-A-1052405, and DE-A-1919181 disclose naphthobenzotriazoles used as optical brighteners.

U.S. Pat. No. 3,793,315 teaches stilbenyl benzotriazole derivatives as optical brighteners. Woessner, et al., in J. Phys. Chem., 1985 (89), 3629-3636 studied the emission of 2-(2-hydroxy-5-methylphenyl)-2H-benzotriazole, and the methoxy analogue thereof.

U.S. Pat. No. 5,486,406 teaches the use of metal complexes of ortho hydroxyphenyl-2H-benzotriazoles in organic light emitting devices.

JP00256667 and JP98140145 disclose metal complexes of ortho hydroxyphenyl-2H-benzotriazoles for use in electroluminescent devices.

Certain 2H-benzotriazole derivatives are found to be suitable for use in organo-electroluminescent devices. In particular, certain 2H-benzotriazole derivatives are suitable blue emitters with good durability.

Accordingly, the present invention relates to 2H-benzotriazole compounds of the formula

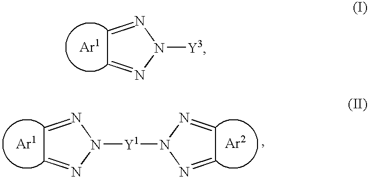

Y¹ is a divalent linking group, and

Y³ is C₁-C₂₆alkyl, especially C₁-C₄alkyl, aryl or heteroaryl, which can optionally be substituted, especially C₆-C₃₀aryl, or C₂-C₂₆heteroaryl, which can optionally be substituted,

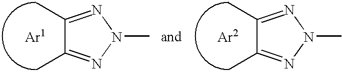

are independently of each other a group of formula

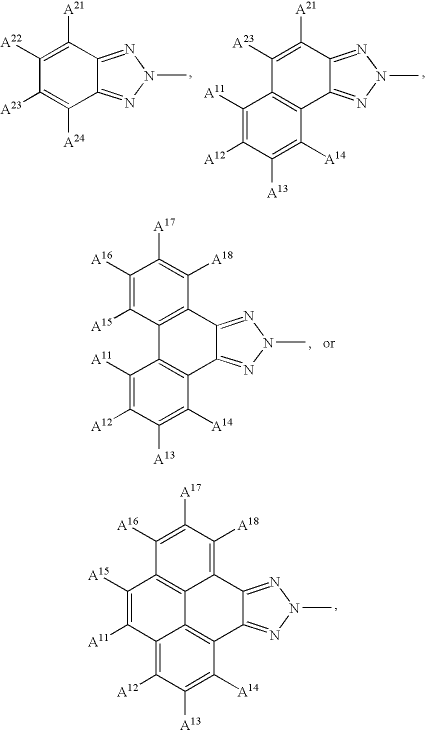

wherein

A²¹, A²², A²³, A²⁴, A¹¹, A¹², A¹³, A¹⁴, A¹⁵, A¹⁶, A¹⁷ and A¹⁸ are independently of each other H, halogen, especially fluorine, hydroxy, C₁-C₂₄alkyl, C₁-C₂₄alkyl which is substituted by E and/or interrupted by D, C₁-C₂₄perfluoroalkyl, C₆-C₁₄perfluoroaryl, especially pentafluorophenyl, C₅-C₁₂cycloalkyl, C₆-C₁₂cycloalkyl which is substituted by G and/or interrupted by S—, —O—, or —NR²⁵—, —NR²⁵R²⁶, C₁-C₂₄alkylthio, —PR³²R³², C₅-C₁₂cycloalkoxy, C₅-C₁₂cycloalkoxy which is substituted by G, C₆-C₂₄aryl, C₆-C₂₄aryl which is substituted by G, C₁-C₂₄alkyl, C₅-C₁₂cycloalkyl, C₇-C₂₅aralkyl, C₁-C₂₄perfluoroalkyl, C₆-C₁₄perfluoroaryl, especially pentafluorophenyl, or C₁-C₂₄haloalkyl; C₂-C₂₀heteroaryl, C₂-C₂₀heteroaryl which is substituted by G, fluorine, C₁-C₂₄alkyl, C₅-C₁₂cycloalkyl, C₇-C₂₅aralkyl, C₁-C₂₄perfluoroalkyl, C₆-C₁₄perfluoroaryl, especially pentafluorophenyl, or C₁-C₂₄haloalkyl; C₂-C₂₄alkenyl, C₂-C₂₄alkynyl, C₁-C₂₄alkoxy, C₁-C₂₄alkoxy which is substituted by E and/or interrupted by D, C₇-C₂₅aralkyl, C₇-C₂₅aralkyl, which is substituted by G, C₇-C₂₅aralkoxy, C₇-C₂₅aralkoxy which is substituted by G, or —CO—R²⁸, or

A²² and A²³ or A¹¹ and A²³ are a group

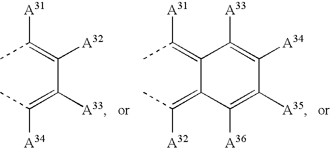

two groups A¹¹, A¹², A¹³, A¹⁴, A¹⁵, A¹⁶, A¹⁷ and A¹⁸, which are neighbouring to each other, are a group

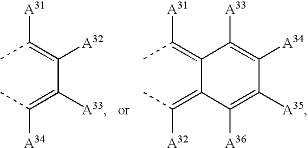

wherein A³¹, A³², A³³, A³⁴, A³⁵ and A³⁶ are independently of each other H, halogen, hydroxy, C₁-C₂₄alkyl, C₁-C₂₄alkyl which is substituted by E and/or interrupted by D, C₁-C₂₄perfluoroalkyl, C₆-C₁₄perfluoroaryl, especially pentafluorophenyl, C₅-C₁₂cycloalkyl, C₅-C₁₂cycloalkyl which is substituted by G and/or interrupted by S—, —O—, or —NR²⁵—, C₅-C₁₂cycloalkoxy, C₅-C₁₂cycloalkoxy which is substituted by G, C₆-C₂₄aryl, C₆-C₂₄aryl which is substituted by G. C₂-C₂₀heteroaryl, C₂-C₂₀heteroaryl which is substituted by G, C₂-C₂₄alkenyl, C₂-C₂₄alkynyl, C₁-C₂₄alkoxy, C₁-C₂₄alkoxy which is substituted by E and/or interrupted by D, C₇-C₂₅aralkyl, C₇-C₂₅aralkyl, which is substituted by G, C₇-C₂₅aralkoxy, C₇-C₂₅aralkoxy which is substituted by G, or —CO—R²⁸, wherein preferably at least one of the substituents A²¹, A²², A₂₃, A²⁴, A¹¹, A¹², A¹³, A¹⁴, A¹⁵, A¹⁶, A¹⁷ and A¹⁸ is C₆-C₂₄aryl which is substituted by fluorine, C₁-C₂₄alkyl, C₆-C₁₂cycloalkyl, C₇-C₂₅aralkyl, C₁-C₂₄perfluoroalkyl, C₆-C₁₄perfluoroaryl, especially pentafluorophenyl, or C₁-C₂₄haloalkyl; or C₂-C₂₆heteroaryl, especially thiophenyl, pyrrolyl, furanyl, benzoxazolyl, or benzothiazolyl, which is substituted by fluorine, C₁-C₂₄alkyl, C₅-C₁₂cycloalkyl, C₇-C₂₅aralkyl, C₁-C₂₄perfluoroalkyl, C₆-C₁₄perfluoroaryl, especially pentafluorophenyl, or C₁-C₂₄haloalkyl, or a group of formula

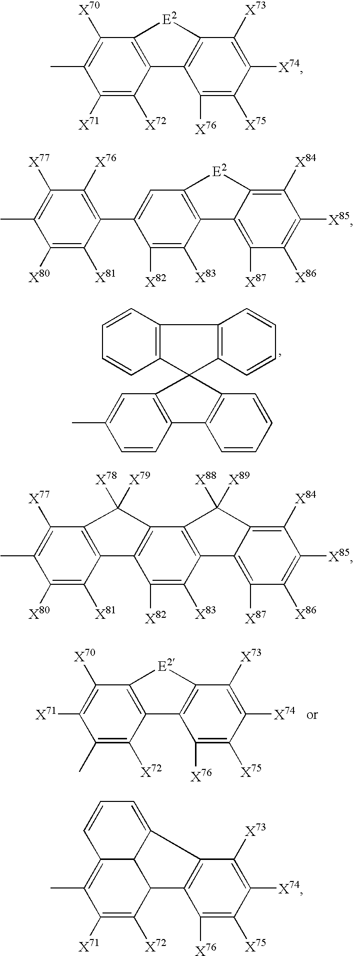

wherein X⁷⁰, X⁷¹, X⁷², X⁷³, X⁷⁴, X⁷⁵, X⁷⁶, X⁷⁷, X⁸⁰, X⁸¹, X⁸², X⁸³, X⁸⁴, X⁸⁵, X⁸⁶, and X⁸⁷ are independently of each other E and/or interrupted by D, C₁-C₂₄perfluoroalkyl, C₆-C₁₄perfluoroaryl, especially pentafluorophenyl, C₅-C₁₂cycloalkyl, C₅-C₁₂cycloalkyl which is substituted by G and/or interrupted by S—, —O—, or —NR²⁵—, —NR²⁵R²⁶, C₁-C₂₄alkylthio, —PR³² R³², C₅-C₁₂cycloalkoxy, C₅-C₁₂cycloalkoxy which is substituted by G, C₆-C₂₄aryl, C₆-C₂₄aryl which is substituted by G, C₁-C₂₄alkyl, C₅-C₁₂cycloalkyl, C₇-C₂₅aralkyl, C₁-C₂₄perfluoroalkyl, C₆-C₁₄perfluoroaryl, especially pentafluorophenyl, or C₁-C₂₄haloalkyl; C₂-C₂₀heteroaryl, C₂-C₂₀heteroaryl which is substituted by G, fluorine, C₁-C₂₄alkyl, C₅-C₁₂cycloalkyl, C₇-C₂₅aralkyl, C₁-C₂₄perfluoroalkyl, C₆-C₁₄perfluoroaryl, especially pentafluorophenyl, or C₁-C₂₄haloalkyl; C₂-C₂₄alkenyl, C₂-C₂₄alkynyl, C₁-C₂₄alkoxy, C₁-C₂₄alkoxy which is substituted by E and/or interrupted by D, C₇-C₂₅aralkyl, C₇-C₂₅aralkyl, which is substituted by G, C₇-C₂₅aralkoxy, C₇-C₂₅aralkoxy which is substituted by G, or —CO—R²⁸, or

two groups X⁷⁰, X⁷¹, X⁷², X⁷³, X⁷⁴, X⁷⁵, X⁷⁶, X⁷⁷, X⁸⁰, X⁸¹, X⁸², X⁸³, X⁸⁴, X⁸⁵, X⁸⁶, and X⁸⁷, which are neighbouring to each other, are a group

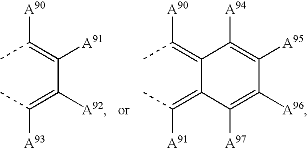

wherein A⁹⁰, A⁹¹, A⁹², A⁹³, A⁹⁴, A⁹⁵, A⁹⁶ and A⁹⁷ are independently of each other H, halogen, especially fluorine, hydroxy, C₁-C₂₄alkyl, C₁-C₂₄alkyl which is substituted by E and/or interrupted by D, C₁-C₂₄perfluoroalkyl, C₆-C₁₄perfluoroaryl, especially pentafluorophenyl, C₅-C₁₂cycloalkyl, C₅-C₁₂cycloalkyl which is substituted by G and/or interrupted by S—, —O—, or —NR²⁵—, C₅-C₁₂cycloalkoxy, C₅-C₁₂cycloalkoxy which is substituted by G, C₆-C₂₄aryl, C₆-C₂₄aryl which is substituted by G, C₂-C₂₀heteroaryl, C₂-C₂₀heteroaryl which is substituted by G, C₂-C₂₄alkenyl, C₂-C₂₄alkynyl, C₁-C₂₄alkoxy, C₁-C₂₄alkoxy which is substituted by E and/or interrupted by D, C₇-C₂₅aralkyl, C₇-C₂₅aralkyl, which is substituted by G, C₇-C₂₅aralkoxy, C₇-C₂₅aralkoxy which is substituted by G, or —CO—R²⁸,

E² is —CR²³═CR²⁴—, especially —CX⁶⁸X⁶⁹—,

E^{2′} is —SiR³⁰R³¹—; —POR³²—; especially —S—, —O—, or —NR²⁵—, wherein R^{25′} is C₁-C₂₄alkyl, or C₆-C₁₀aryl,

X⁶⁸, X⁶⁹, X⁷⁸, X⁷⁹, X⁸⁸ and X⁸⁹ are independently of each other C₁-C₁₈ alkyl, C₁-C₂₄alkyl which is substituted by E and/or interrupted by D, C₆-C₂₄aryl, C₆-C₂₄aryl which is substituted by G, C₂-C₂₀heteroaryl, C₂-C₂₀heteroaryl which is substituted by G, C₂-C₂₄alkenyl, C₂-C₂₄alkynyl, C₁-C₂₄alkoxy, C₁-C₂₄alkoxy which is substituted by E and/or interrupted by D, or C₇-C₂₅aralkyl, or

X⁷⁸ and X⁷⁹, and/or X⁸⁸ and X⁸⁹ form a ring, especially a five- or six-membered ring, or

X⁶⁸ and X⁷⁰, X⁶⁹ and X⁷³, X⁷⁷ and X⁷⁸ and/or X⁸⁴ and X⁸⁹ are a group

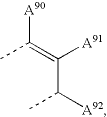

D is —CO—; —COO—; —S—; —SO—; —SO₂—; —O—; —NR²⁵—; —SiR³⁰R³¹—; —POR³²—; —CR²³═CR²⁴—; or —C≡C—; and

E is —OR²⁹; —SR; —NR²⁵R²⁶; —COR²⁸; —COOR²⁷; —CONR²⁵R²⁶; —CN; —OCOOR²⁷; or halogen;

G is E, or C₁-C₂₄alkyl, wherein

R²³, R²⁴, R²⁵ and R²⁶ are independently of each other H; C₆-C₁₈aryl; C₆-C₁₈aryl which is substituted by C₁-C₂₄alkyl, or C₁-C₂₄alkoxy; C₁-C₂₄alkyl; or C₁-C₂₄alkyl which is interrupted by —O—; or

R²⁵ and R²⁶ together form a five or six membered ring, in particular

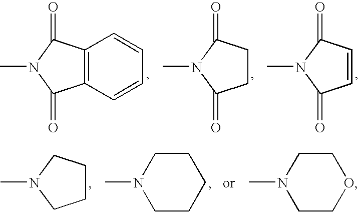

R²⁷ and R²⁸ are independently of each other H; C₆-C₁₈aryl; C₆-C₁₈aryl which is substituted by C₁-C₂₄alkyl, or C₁-C₂₄alkoxy; C₁-C₂₄alkyl; or C₁-C₂₄alkyl which is interrupted by —O—,

R²⁹ is H; C₆-C₁₈aryl; C₆-C₁₈aryl, which is substituted by C₁-C₂₄alkyl, or C₁-C₂₄alkoxy; C₁-C₂₄alkyl; or C₁-C₂₄alkyl which is interrupted by —O—,

R³⁰ and R³¹ are independently of each other C₁-C₂₄alkyl, C₆-C₁₈aryl, or C₆-C₁₈aryl, which is substituted by C₁-C₂₄alkyl, and

R³² is C₁-C₂₄alkyl, C₆-C₁₈aryl, or C₆-C₁₈aryl, which is substituted by C₁-C₂₄alkyl.

In a preferred embodiment of the present invention at least one of the substituents A²¹, A²², A²³, A²⁴, A¹¹, A¹², A¹³, A¹⁴, A¹⁵, A¹⁶, A¹⁷ and A¹⁸, especially A¹², A²¹ and/or A²³, are a group of formula

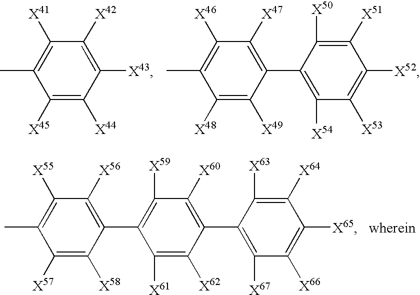

X⁴¹, X⁴², X⁴³, X⁴⁴, X⁴⁵, X⁴⁶, X⁴⁷, X⁴⁸, X⁴⁹, X⁵⁰, X⁵¹, X⁵², X⁵³, X⁵⁴, X⁵⁵, X⁵⁶, X⁵⁷, X⁵⁸, X⁵⁹, X⁶⁰, X⁶¹, X⁶², X⁶³, X⁶⁴, X⁶⁵, X⁶⁶ and X⁶⁷ are independently of each other H, fluorine, —NR²⁵R²⁶, C₁-C₂₄alkyl, C₅-C₁₂cycloalkyl, C₇-C₂₅aralkyl, C₁-C₂₄perfluoroalkyl, C₆-C₁₄perfluoroaryl, especially pentafluorophenyl, or C₁-C₂₄haloalkyl, C₁-C₂₄alkyl, which is optionally substituted by E and/or interrupted by D, C₁-C₂₄alkenyl, which is optionally substituted by E, C₅-C₁₂cycloalkyl, which is optionally substituted by G, C₅-C₁₂cycloalkoxy, which is optionally substituted by G, C₆-C₁₈aryl, which is optionally substituted by G, C₁-C₂₄alkoxy, which is optionally substituted by E and/or interrupted by D, C₆-C₁₈aryloxy, which is optionally substituted by G, C₇-C₁₈arylalkoxy, which is optionally substituted by G, C₁-C₂₄alkylthio, which is optionally substituted by E and/or interrupted by D, C₂-C₂₀heteroaryl which is substituted by G, or C₆-C₁₈aralkyl, which is optionally substituted by G, or

X⁴³, X⁶⁵ or X⁵² are a group of formula

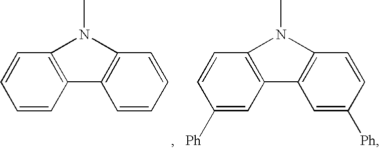

two groups X⁴¹, X⁴², X⁴³, X⁴⁴, X⁴⁵, X⁴⁶, X⁴⁷, X⁴⁸, X⁴⁹, X⁵⁰, X⁵¹, X⁵², X⁵³, X⁵⁴, X⁵⁵, X⁵⁶, X⁵⁷, X⁵⁸, X⁵⁹, X⁶⁰, X⁶¹, X⁶², X⁶³, X⁶⁴, X⁶⁵, X⁶⁶ and X⁶⁷, which are neighbouring to each other, are a group

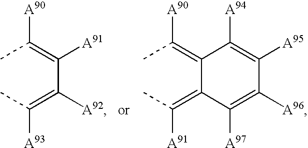

wherein A⁹⁰, A⁹¹, A⁹², A⁹³, A⁹⁴, A⁹⁵, A⁹⁶ and A⁹⁷ are independently of each other H, halogen, hydroxy, C₁-C₂₄alkyl, C₁-C₂₄alkyl which is substituted by E and/or interrupted by D, C₁-C₂₄perfluoroalkyl, C₆-C₁₄perfluoroaryl, especially pentafluorophenyl, C₅-C₁₂cycloalkyl, C₅-C₁₂cycloalkyl which is substituted by G and/or interrupted by S—, —O—, or —NR²⁵—, C₅-C₁₂cycloalkoxy, C₅-C₁₂cycloalkoxy which is substituted by G, C₆-C₂₄aryl, C₆-C₂₄aryl which is substituted by G, C₂-C₂₀heteroaryl, C₂-C₂₀heteroaryl which is substituted by G, C₂-C₂₄alkenyl, C₂-C₂₄alkynyl, C₁-C₂₄alkoxy, C₁-C₂₄alkoxy which is substituted by E and/or interrupted by D, C₇-C₂₅aralkyl, C₇-C₂₅aralkyl, which is substituted by G, C₇-C₂₅aralkoxy, C₇-C₂₅aralkoxy which is substituted by E, or —CO—R²⁸, wherein R²⁵, R²⁶ and R²⁸, D, E and G are as defined above and preferably at least one of the substituents X⁴¹, X⁴², X⁴³, X⁴⁴, X⁴⁵, X⁴⁶, X⁴⁷, X⁴⁸, X⁴⁹, X⁵⁰, X⁵¹, X⁵², X⁵³, X⁵⁴, X⁵⁵, X⁵⁶, X⁵⁷, X⁵⁸, X⁵⁹, X⁶⁰, X⁶¹, X⁶², X⁶³, X⁶⁴, X⁶⁵, X⁶⁶ and X⁶⁷ is fluorine, —NR²⁵R²⁶, C₁-C₂₄alkyl, C₅-C₁₂cycloalkyl, C₇-C₂₅aralkyl, C₁-C₂₄perfluoroalkyl, C₆-C₁₄perfluoroaryl, especially pentafluorophenyl, or C₁-C₂₄haloalkyl.

In another preferred embodiment of the present invention at least one of the substituents A²¹, A²², A²³, A²⁴, A¹¹, A¹², A¹³, A¹⁴, A¹⁵, A¹⁶, A¹⁷ and A¹⁸, especially A¹² and/or A²³ are a group of formula

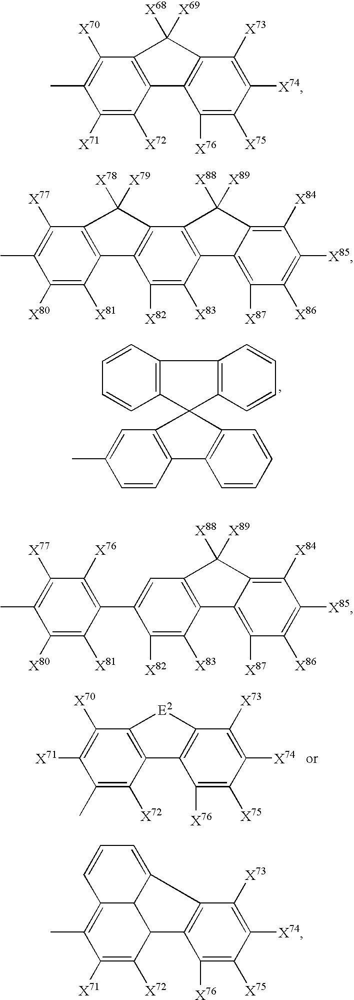

wherein

X⁶⁸, X⁶⁹, X⁷⁸, X⁷⁹, X⁸⁸ and X⁸⁹ are independently of each other C₁-C₂₄alkyl, especially C₁-C₁₂alkyl, which can be interrupted by one or two oxygen atoms,

X⁷⁰, X⁷¹, X⁷², X⁷³, X⁷⁴, X⁷⁵, X⁷⁶, X⁷⁷, X⁸⁰, X⁸¹, X⁸², X⁸³, X⁸⁴, X⁸⁵, X⁸⁶ and X⁸⁷ are independently of each other H, CN, C₁-C₂₄alkyl, C₆-C₁₀aryl, C₁-C₂₄alkoxy, C₁-C₂₄alkylthio, —NR²⁵R²⁶, —CONR²⁵R²⁶, or —COOR²⁷, wherein

R²⁵ and R²⁶ are independently of each other H, C₆-C₁₈aryl, C₇-C₁₈aralkyl, or C₁-C₂₄alkyl, and

R²⁷ is C₁-C₂₄alkyl, or

R²⁵ and R²⁶ together form a five or six membered ring, in particular

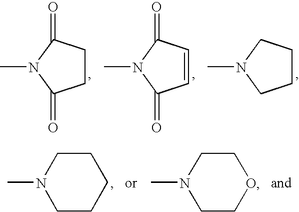

E² is —S—, —O—, or —NR^{25′}—, wherein R^{25′} is C₁-C₂₄alkyl, or C₆-C₁₀aryl.

The 2H-benzotriazole compound or compounds should emit light below about 520 nm, especially between about 380 nm and about 520 nm. The 2H-benzotriazole compound or compounds should have a NTSC coordinate of between about (0.12, 0.05) and about (0.16, 0.10), especially a NTSC coordinate of about (0.14, 0.08).

The 2H-benzotriazole compound or compounds should have a melting point above about 150° C., especially above about 200° C., more preferred above about 250° C., most preferred above about 300° C.

Preferably, Y³ is a group of formula

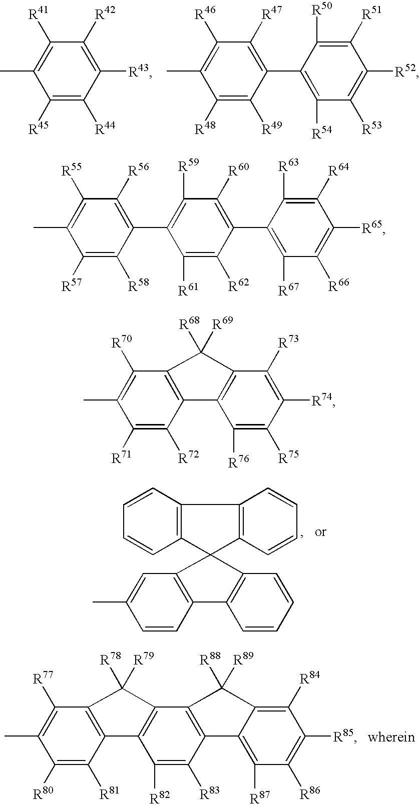

R⁴¹, R⁴², R⁴³, R⁴⁴, R⁴⁵, R⁴⁶, R⁴⁷, R⁴⁸, R⁴⁹, R⁵⁰, R⁵¹, R⁵², R⁵³, R⁵⁴, R⁵⁵, R⁵⁶, R⁵⁷, R⁵⁸, R⁵⁹, R⁶⁰, R⁶¹, R⁶², R⁶³, R⁶⁴, R⁶⁵, R⁶⁶, R⁶⁷, R⁷⁰, R⁷¹, R⁷², R⁷³, R⁷⁴, R⁷⁵, R⁷⁶, R⁷⁷, R⁸⁰, R⁸¹, R⁸², R⁸³ , R⁸⁴, R⁸⁵, R⁸⁶, and R⁸⁷ are independently of each other H, fluorine, C₁-C₂₄perfluoroalkyl, C₆-C₁₄perfluoroaryl, especially pentafluorophenyl, —NR²⁵R²⁶, C₁-C₂₄alkyl, which is optionally substituted by E and/or interrupted by D, C₁-C₂₄alkenyl, which is optionally substituted by E, C₅-C₁₂cycloalkyl, which is optionally substituted by G, C₅-C₁₂cycloalkoxy, which is optionally substituted by G, C₆-C₁₈aryl, which is optionally substituted by G, C₁-C₂₄alkoxy, which is optionally substituted by E and/or interrupted by D, C₆-C₁₈aryloxy, which is optionally substituted by G, C₇-C₁₈arylalkoxy, which is optionally substituted by G, C₁-C₂₄alkylthio, which is optionally substituted by E and/or interrupted by D, C₂-C₂₀heteroaryl which is substituted by G, or C₆-C₁₈aralkyl, which is optionally substituted by G, or

R⁴³, R⁶⁵ or R⁵² are a group of formula

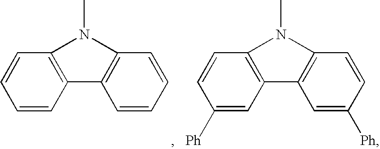

two groups R⁴¹, R⁴², R⁴³, R⁴⁴, R⁴⁵, R⁴⁶, R⁴⁷, R⁴⁸, R⁴⁹, R⁵⁰, R⁵¹, R⁵², R⁵³, R⁵⁴, R⁵⁵, R⁵⁶, R⁵⁷, R⁵⁸, R⁵⁹, R⁶⁰, R⁶¹, R⁶², R⁶³, R⁶⁴, R⁶⁵, R⁶⁶, R⁶⁷, R⁷⁰, R⁷¹, R⁷², R⁷³, R⁷⁴, R⁷⁵, R⁷⁶, R⁷⁷, R⁸⁰, R⁸¹, R⁸², R⁸³, R⁸⁴, R⁸⁵, R⁸⁶, and R⁸⁷, which are neighbouring to each other, are a group

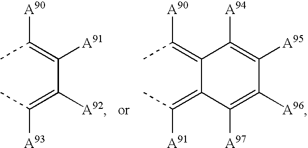

wherein A⁹⁰, A⁹¹, A⁹², A⁹³, A⁹⁴, A⁹⁵, A⁹⁶ and A⁹⁷ are independently of each other H, halogen, especially fluorine, —NR²⁵R²⁶, hydroxy, C₁-C₂₄alkyl, C₁-C₂₄alkyl which is substituted by E and/or interrupted by D, C₁-C₂₄perfluoroalkyl, C₆-C₁₄perfluoroaryl, especially pentafluorophenyl, C₅-C₁₂cycloalkyl, C₅-C₁₂cycloalkyl which is substituted by G and/or interrupted by S—, —O—, or —NR²⁵—, C₅-C₁₂cycloalkoxy, C₅-C₁₂cycloalkoxy which is substituted by G, C₆-C₂₄aryl, C₆-C₂₄aryl which is substituted by G, C₂-C₂₀heteroaryl, C₂-C₂₀heteroaryl which is substituted by G, C₂-C₂₄alkenyl, C₂-C₂₄alkynyl, C₁-C₂₄alkoxy, C₁-C₂₄alkoxy which is substituted by E and/or interrupted by D, C₇-C₂₅aralkyl, C₇-C₂₅aralkyl, which is substituted by G, C₇-C₂₅aralkoxy, C₇-C₂₅aralkoxy which is substituted by G, or —CO—R²⁸, R⁶⁸, R⁶⁹, R⁷⁸, R⁷⁹, R⁸⁸ and R⁸⁹ are independently of each other C₁-C₁₈ alkyl, C₁-C₂₄alkyl which is substituted by E and/or interrupted by D, C₆-C₂₄aryl, C₆-C₂₄aryl which is substituted by G, C₂-C₂₀heteroaryl, C₂-C₂₀heteroaryl which is substituted by G, C₂-C₂₄alkenyl, C₂-C₂₄alkynyl, C₁-C₂₄alkoxy, C₁-C₂₄alkoxy which is substituted by E and/or interrupted by D, or C₇-C₂₅aralkyl, or

R⁶⁸ and R⁶⁹, R⁷⁸ and R⁷⁹, and/or R⁸⁸ and R⁸⁹ form a ring, especially a five- or six-membered ring, or

R⁶⁸ and R⁷⁰, R⁶⁹ and R⁷³, R⁷⁷ and R⁷⁸ and/or R⁸⁴ and R⁸⁹ are a group

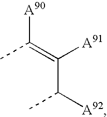

D is —CO—; —COO—; —S—; —SO—; —SO₂—; —O—; —NR²⁵—; —SiR³⁰R³¹—; —POR³²—; —CR²³═CR²⁴—; or —C≡C—; and

E is —OR²⁹; —SR²⁹; —NR²⁵R²⁶; —COR²⁸; —COOR²⁷; —CONR²⁵R²⁶; —CN; —OCOOR²⁷; or halogen; G is E, or C₁-C₂₄alkyl; wherein

R²³, R²⁴, R²⁵ and R²⁶ are independently of each other H; C₆-C₁₈aryl; C₆-C₁₈aryl which is substituted by C₁-C₂₄alkyl, or C₁-C₂₄alkoxy; C₁-C₂₄alkyl; or C₁-C₂₄alkyl which is interrupted by —O—; or

R²⁵ and R²⁶ together form a five or six membered ring, in particular

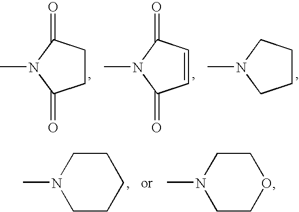

R²⁷ and R²⁸ are independently of each other H; C₆-C₁₈aryl; C₆-C₁₈aryl which is substituted by C₁-C₂₄alkyl, or C₁-C₂₄alkoxy; C₁-C₂₄alkyl; or C₁-C₂₄alkyl which is interrupted by —O—,

R²⁹ is H; C₆-C₁₈aryl; C₆-C₁₈aryl, which is substituted by C₁-C₂₄alkyl, or C₁-C₂₄alkoxy; C₁-C₂₄alkyl; or C₁-C₂₄alkyl which is interrupted by —O—,

R³⁰ and R³¹ are independently of each other C₁-C₂₄alkyl, C₆-C₁₈aryl, or C₆-C₁₈aryl, which is substituted by C₁-C₂₄alkyl, and

R³² is C₁-C₂₄alkyl, C₆-C₁₈aryl, or C₆-C₁₈aryl, which is substituted by C₁-C₂₄alkyl, or

R⁴³, or R⁵² are a group of formula

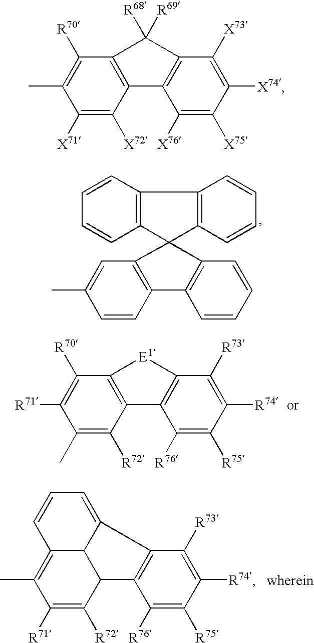

R^{68′} and R^{69′} are independently of each other C₁-C₂₄alkyl, especially C₁-C₁₂alkyl, which can be interrupted by one or two oxygen atoms,

R^{70′}, R^{71′}, R^{72′}, R^{73′}, R^{74′}, R^{75′} and R^{76′} are independently of each other H, CN, C₁-C₂₄alkyl, C₆-C₁₀aryl, C₁-C₂₄alkoxy, C₁-C₂₄alkylthio, —NR^{25′}R^{26′}, —CONR^{25′}R^{26′}, or —COOR^{27′,}

R^{25′} and R^{26′} are independently of each other H, C₆-C₁₈aryl, C₇-C₁₈aralkyl, or C₁-C₂₄alkyl, and

R^{27′} is C₁-C₂₄alkyl; and

E^{1′} is —S—, —O—, or —NR²⁵—, wherein R^{25′} is C₁-C₂₄alkyl, or C₆-C₁₀aryl.

Y¹ is preferably a group of formula

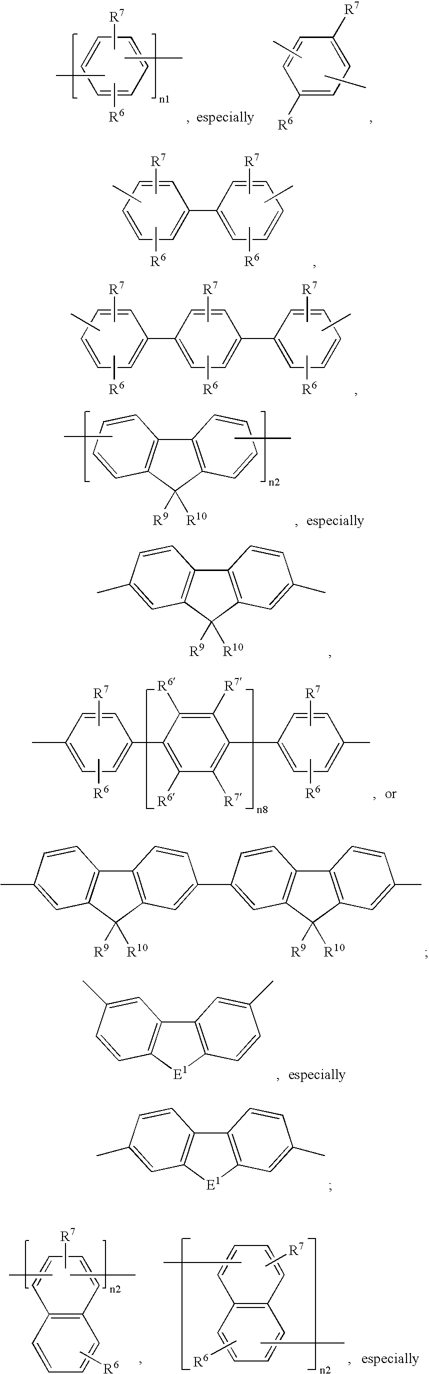

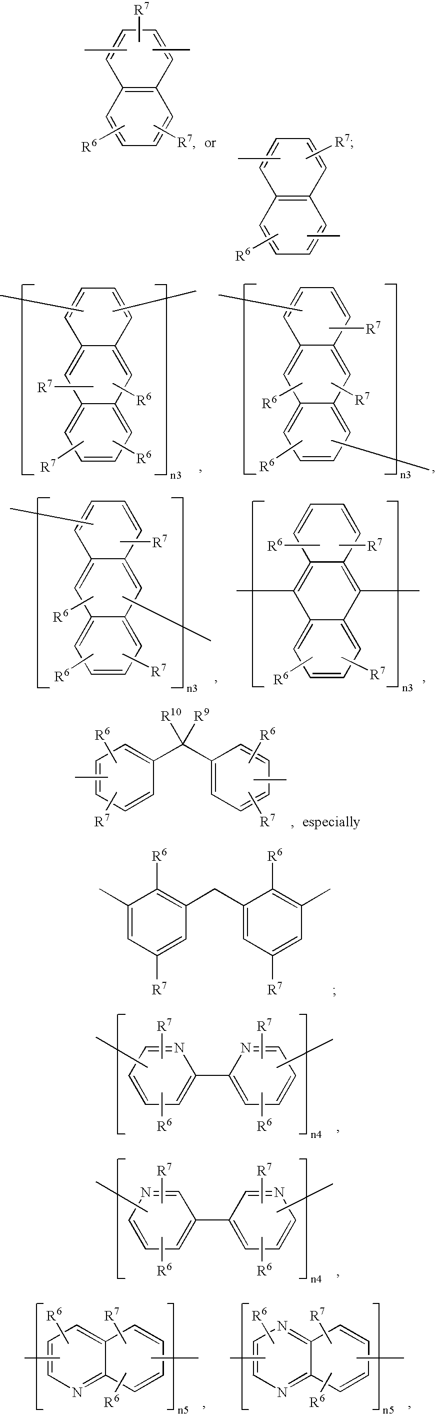

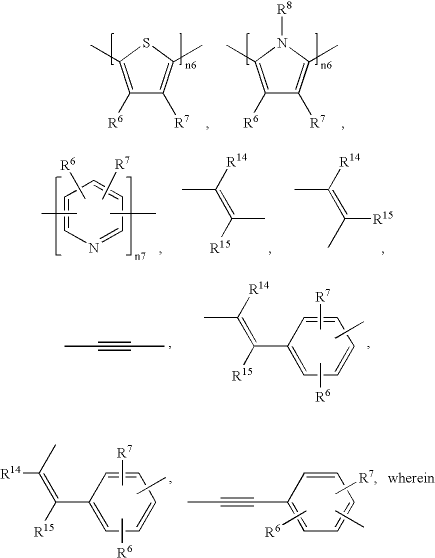

or

n1, n2, n3, n4, n5, n6, n7 and n8 are 1, 2, or 3, in particular 1,

E¹ is —S—, —O—, or —NR^{25′}—, wherein R^{25′} is C₁-C₂₄alkyl, or C₆-C₁₀aryl,

R⁶ and R⁷ are independently of each other H, halogen, especially fluorine, —NR²⁵R²⁶, hydroxy, C₁-C₂₄alkyl, C₁-C₂₄alkyl which is substituted by E and/or interrupted by D, C₁-C₂₄perfluoroalkyl, C₆-C₁₄perfluoroaryl, especially pentafluorophenyl, C₅-C₁₂cycloalkyl, C₅-C₁₂cycloalkyl which is substituted by G and/or interrupted by S—, —O—, or —NR²⁵—, C₅-C₁₂cycloalkoxy, C₅-C₁₂cycloalkoxy which is substituted by G, C₆-C₂₄aryl, C₆-C₂₄aryl which is substituted by G, C₂-C₂₀heteroaryl, C₂-C₂₀heteroaryl which is substituted by G, C₂-C₂₄alkenyl, C₂-C₂₄alkynyl, C₁-C₂₄alkoxy, C₁-C₂₄alkoxy which is substituted by E and/or interrupted by D, C₇-C₂₅aralkyl, C₇-C₂₅aralkyl, which is substituted by G, C₇-C₂₅aralkoxy, C₇-C₂₅aralkoxy which is substituted by G, or —CO—R²⁸,

R^{6′} and R^{7′} have the meaning of R⁶, or together form a group

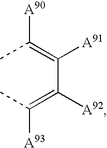

wherein A⁹⁰, A⁹¹, A⁹², and A⁹³ are independently of each other H, halogen, hydroxy, C₁-C₂₄alkyl, C₁-C₂₄alkyl which is substituted by E and/or interrupted by D, C₁-C₂₄perfluoroalkyl, C₆-C₁₄perfluoroaryl, especially pentafluorophenyl, C₅-C₁₂cycloalkyl, C₅-C₁₂cycloalkyl which is substituted by G and/or interrupted by S—, —O—, or —NR²⁵—, C₅-C₁₂cycloalkoxy, C₆-C₁₂cycloalkoxy which is substituted by G, C₆-C₂₄aryl, C₆-C₂₄aryl which is substituted by G, C₂-C₂₀heteroaryl, C₂-C₂₀heteroaryl which is substituted by G, C₂-C₂₄alkenyl, C₂-C₂₄alkynyl, C₁-C₂₄alkoxy, C₁-C₂₄alkoxy which is substituted by E and/or interrupted by D, C₇-C₂₅aralkyl, C₇-C₂₅aralkyl, which is substituted by G, C₇-C₂₅aralkoxy, C₇-C₂₅aralkoxy which is substituted by E, or —CO—R²⁸,

R⁸ is C₁-C₂₄alkyl, C₁-C₂₄alkyl which is substituted by E and/or interrupted by D, C₆-C₂₄ aryl, or C₇-C₂₅aralkyl,

R⁹ and R¹⁰ are independently of each other C₁-C₂₄alkyl, C₁-C₂₄alkyl which is substituted by E and/or interrupted by D, C₆-C₂₄aryl, C₆-C₂₄aryl which is substituted by G, C₂-C₂₀heteroaryl, C₂-C₂₀heteroaryl which is substituted by G, C₂-C₂₄alkenyl, C₂-C₂₄alkynyl, C₁-C₂₄alkoxy, C₁-C₂₄alkoxy which is substituted by E and/or interrupted by D, or C₇-C₂₅aralkyl, or

R⁹ and R¹⁰ form a ring, especially a five- or six-membered ring,

R¹⁴ and R¹⁵ are independently of each other H, C₁-C₂₄alkyl, C₁-C₂₄alkyl which is substituted by E and/or interrupted by D, C₆-C₂₄aryl, C₆-C₂₄aryl which is substituted by G, C₂-C₂₀heteroaryl, or C₂-C₂₀heteroaryl which is substituted by G,

D is —CO—, —COO—, —S—, —SO—, —SO₂—, —O—, —NR²⁵—, —SiR³⁰R³¹—, —POR³²—, —CR²³═CR²⁴—, or —C≡C—,

G is E, or C₁-C₂₄alkyl, and

E is —OR²⁹, —SR²⁹, —NR²⁵R²⁶, —COR²⁸, —COOR²⁷, —CONR²⁵R²⁶, —CN, —OCOOR²⁷, or halogen, wherein

R²³, R²⁴, R²⁵ and R²⁶ are independently of each other H, C₆-C₁₈aryl, C₆-C₁₈aryl which is substituted by C₁-C₂₄alkyl, C₁-C₂₄alkoxy, C₁-C₂₄alkyl, or C₁-C₂₄alkyl which is interrupted by —O—, or

R²⁵ and R²⁶ together form a five or six membered ring, in particular

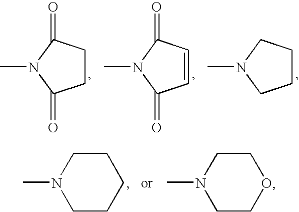

R²⁷ and R²⁸ are independently of each other H, C₆-C₁₈aryl, C₆-C₁₈aryl which is substituted by C₁-C₂₄alkyl, or C₁-C₂₄alkoxy, C₁-C₂₄alkyl, or C₁-C₂₄alkyl which is interrupted by —O—,

R²⁹ is H, C₆-C₁₈aryl, C₆-C₁₈aryl, which is substituted by C₁-C₂₄alkyl, C₁-C₂₄alkoxy, C₁-C₂₄alkyl, or C₁-C₂₄alkyl which is interrupted by —O—,

R³⁰ and R³¹ are independently of each other C₁-C₂₄alkyl, C₆-C₁₈aryl, or C₆-C₁₈aryl, which is substituted by C₁-C₂₄alkyl, and

R³² is C₁-C₂₄alkyl, C₆-C₁₈aryl, or C₆-C₁₈aryl, which is substituted by C₁-C₂₄alkyl.

In a preferred embodiment the present invention is directed to 2H-benzotriazole compounds of formula

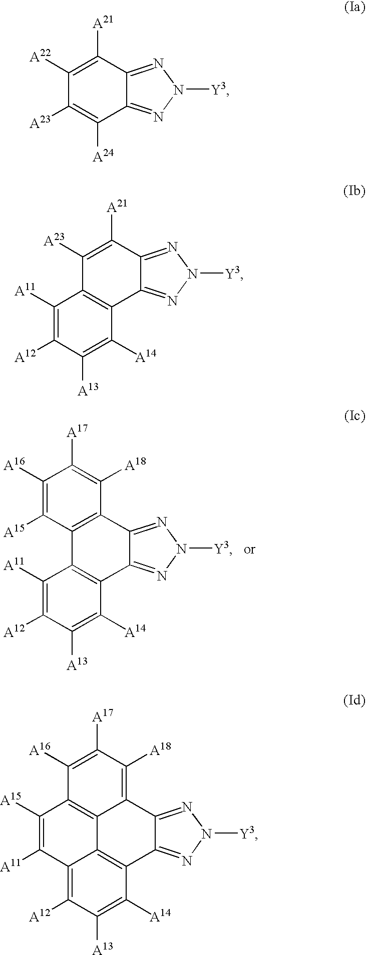

wherein A¹² or A²³ are a group of formula

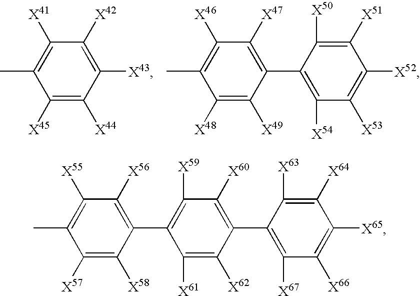

wherein X⁴¹, X⁴², X⁴³, X⁴⁴, X⁴⁵, X⁴⁶, X⁴⁷, X⁴⁸, X⁴⁹, X⁵⁰, X⁵¹, X⁵², X⁵³, X⁵⁴, X⁵⁵, X⁵⁶, X⁵⁷, X⁵⁸, X⁵⁹, X⁶⁰, X⁶¹, X⁶², X⁶³, X⁶⁴, X⁶⁵, X⁶⁶, and X⁶⁷ are independently of each other are independently of each other H, fluorine, CN, C₁-C₂₄alkyl, C₅-C₁₂cycloalkyl, C₇-C₂₅aralkyl, C₁-C₂₄perfluoroalkyl, C₆-C₁₄perfluoroaryl, especially pentafluorophenyl, C₁-C₂₄haloalkyl, C₆-C₁₀aryl, which can optionally be substituted by one, or more C₁-C₈alkyl, or C₁-C₈alkoxy groups; C₁-C₂₄alkoxy, C₁-C₂₄alkylthio, —NR²⁵R²⁶, —CONR²⁵R²⁶, or —COOR²⁷, or

X⁴³, X⁶⁵ or X⁵² are a group of formula

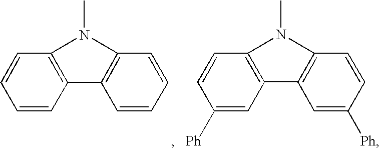

or

two groups X⁴¹, X⁴², X⁴³, X⁴⁴, X⁴⁵, X⁴⁶, X⁴⁷, X⁴⁸, X⁴⁹, X⁵⁰, X⁵¹, X⁵², X⁵³, X⁵⁴, X⁵⁵, X⁵⁶, X⁵⁷, X⁵⁸, X⁵⁹, X⁶⁰, X⁶¹, X⁶², X⁶³, X⁶⁴, X⁶⁵, X⁶⁶ and X⁶⁷, which are neighbouring to each other, are a group

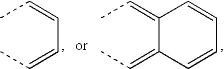

wherein preferably at least one of the substituents X⁴¹, X⁴², X⁴³, X⁴⁴, X⁴⁵, X⁴⁶, X⁴⁷, X⁴⁸, X⁴⁹, X⁵⁰, X⁵¹, X⁵², X⁵³, X⁵⁴, X⁵⁵, X⁵⁶, X⁵⁷, X⁵⁸, X⁵⁹, X⁶⁰, X⁶¹, X⁶², X⁶³, X⁶⁴, X⁶⁵, X⁶⁶ and X⁶⁷ is fluorine, —NR²⁵R²⁶, C₁-C₂₄alkyl, C₅-C₁₂cycloalkyl, C₇-C₂₅aralkyl, C₁-C₂₄perfluoroalkyl, C₆-C₁₄perfluoroaryl, especially pentafluorophenyl, or C₁-C₂₄haloalkyl, or A¹² and A²³ are a group of formula

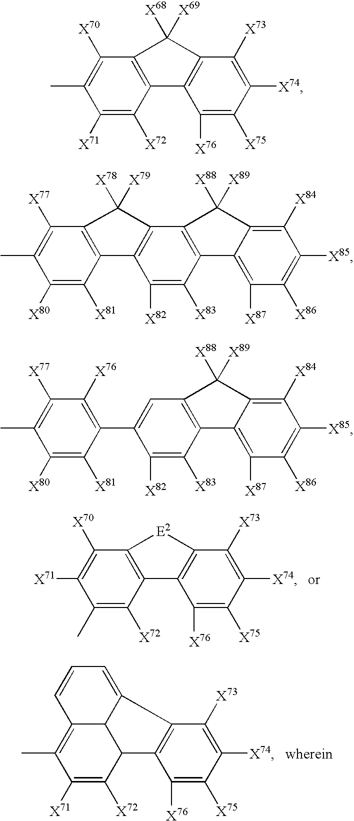

X⁶⁸, X⁶⁹, X⁷⁸, X⁷⁹, X⁸⁸ and X⁸⁹ are independently of each other C₁-C₂₄alkyl, especially C₁-C₁₂alkyl, which can be interrupted by one or two oxygen atoms,

X⁷⁰, X⁷¹, X⁷², X⁷³, X⁷⁴, X⁷⁵, X⁷⁶, X⁷⁷, X⁸⁰, X⁸¹, X⁸², X⁸³, X⁸⁴, X⁸⁵, X⁸⁶ and X⁸⁷ are independently of each other H, CN, C₁-C₂₄alkyl, C₆-C₁₀aryl, which can optionally be substituted by one, or more C₁-C₈alkyl, or C₁-C₈alkoxy groups; C₁-C₂₄alkoxy, C₁-C₂₄alkylthio, —NR²⁵R²⁶, —CONR²⁵R²⁶, or —COOR²⁷,

E² is —S—, —O—, or —NR^{25′}—, wherein R^{25′} is C₁-C₂₄alkyl, or C₆-C₁₀aryl,

A²¹, A²² and A²⁴ are independently of each other hydrogen, halogen, especially fluorine, C₁-C₂₄alkyl, C₁-C₂₄perfluoroalkyl, C₆-C₁₄perfluoroaryl, especially pentafluorophenyl, C₅-C₁₂cycloalkyl, C₇-C₂₅aralkyl, C₁-C₂₄haloalkyl, C₆-C₁₈aryl, which can optionally be substituted by one, or more C₁-C₈alkyl, or C₁-C₈alkoxy groups; —NR²⁵R²⁶, —CONR²⁵R²⁶, or —COOR²⁷, or C₂-C₁₀heteroaryl, especially a group of formula

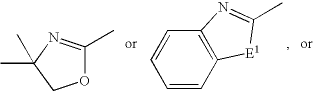

A²² and A²³ or A¹¹ and A²³ are a group of formula

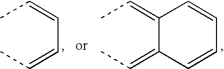

A¹¹, A¹³, A¹⁴, A¹⁵, A¹⁶, A¹⁷, and A¹⁸ are independently of each other H, CN, C₁-C₂₄alkyl, C₅-C₁₂cycloalkyl, C₇-C₂₅aralkyl, C₁-C₂₄perfluoroalkyl, C₆-C₁₄perfluoroaryl, especially pentafluorophenyl, C₁-C₂₄haloalkyl, C₁-C₂₄alkoxy, C₁-C₂₄alkylthio, C₆-C₁₈aryl, —NR²⁵R²⁶, —CONR²⁵R²⁶, or —COOR²⁷, or C₂-C₁₀heteroaryl, wherein

R²⁵ and R²⁶ are independently of each other H, C₆-C₁₈aryl, C₇-C₁₈aralkyl, or C₁-C₂₄alkyl, R²⁷ is C₁-C₂₄alkyl, and

Y³ is a group of formula

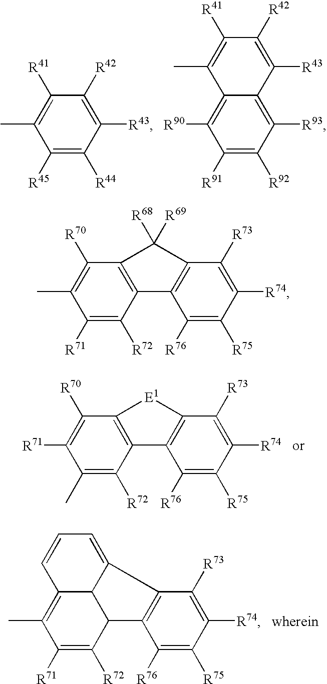

R⁴¹ is hydrogen, C₁-C₂₄alkoxy, or —OC₇-C₁₈aralkyl,

R⁴² is hydrogen, or C₁-C₂₄alkyl,

R⁴³ is hydrogen, halogen, —CONR²⁵R²⁶, —COOR²⁷,

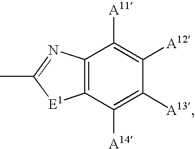

especially

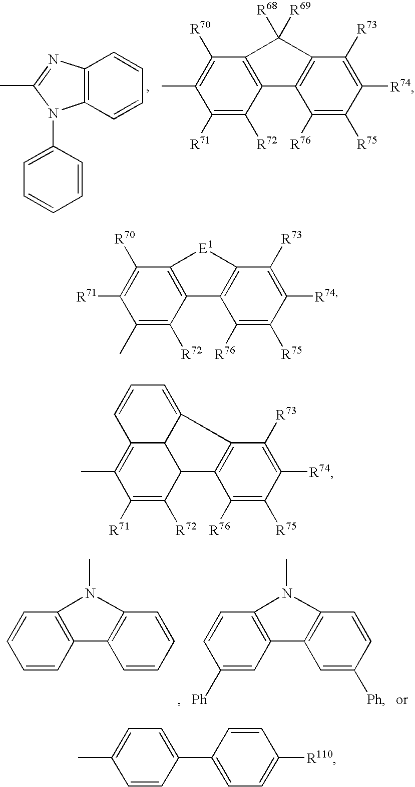

wherein

A^{11′}, A^{12′}, A^{13′}, and A^{14′} are independently of each other H, CN, C₁-C₂₄alkyl, C₁-C₂₄alkoxy, C₁-C₂₄alkylthio, —NR²⁵R²⁶, CONR²⁵R²⁶, or —COOR²⁷,

E¹ is —S—, —O—, or —NR^{25′}—, wherein R^{25′} is C₁-C₂₄alkyl, or C₆-C₁₀aryl,

R¹¹⁰ is H, CN, C₁-C₂₄alkyl, C₁-C₂₄alkoxy, C₁-C₂₄alkylthio, —NR²⁵R²⁶, —CONR²⁵R²⁶, or —COOR²⁷, or

R⁴² and R⁴³ are a group of formula

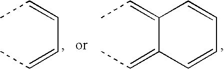

R⁴⁴ is hydrogen, or C₁-C₂₄alkyl,

R⁴⁵ is hydrogen, or C₁-C₂₄alkyl,

R⁶⁸ and R⁶⁹ are independently of each other C₁-C₂₄alkyl, especially C₁-C₁₂alkyl, which can be interrupted by one or two oxygen atoms,

R⁷⁰, R⁷¹, R⁷², R⁷³, R⁷⁴, R⁷⁵, R⁷⁶, R⁹⁰, R⁹¹, R⁹², and R⁹³ are independently of each other H, CN, C₁-C₂₄alkyl, C₆-C₁₀aryl, C₁-C₂₄alkoxy, C₁-C₂₄alkylthio, —NR²⁵R²⁶, —CONR²⁵R²⁶, or —COOR²⁷,

R²⁵ and R²⁶ are independently of each other H, C₆-C₁₈aryl, C₇-C₁₈aralkyl, or C₁-C₂₄alkyl, and

R²⁷ is C₁-C₂₄alkyl.

In a preferred embodiment the present invention is directed to 2H-benzotriazole compounds of formula

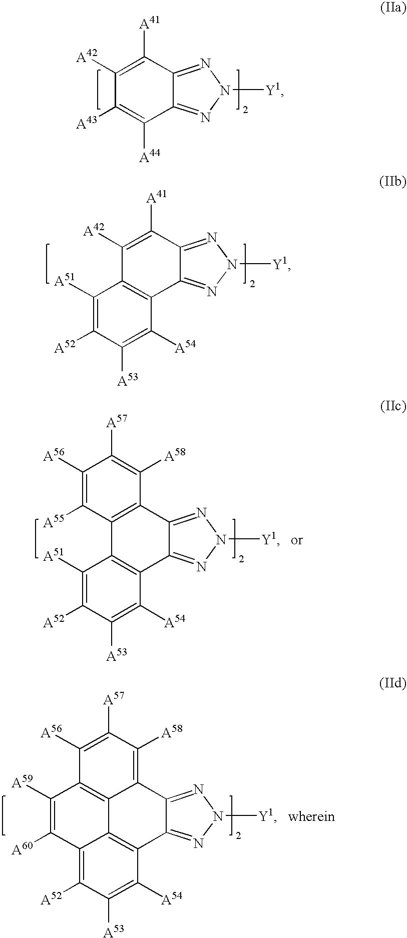

wherein A⁵² and A⁴³ are a group of formula

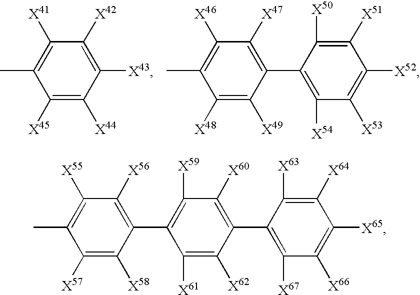

wherein X⁴¹, X⁴², X⁴³, X⁴⁴, X⁴⁵, X⁴⁶, X⁴⁷, X⁴⁸, X⁴⁹, X⁵⁰, X⁵¹, X⁵², X⁵³, X⁵⁴, X⁵⁵, X⁵⁶, X⁵⁷, X⁵⁸, X⁵⁹, X⁶⁰, X⁶¹, X⁶², X⁶³, X⁶⁴, X⁶⁵, X⁶⁶ and X⁶⁷ are independently of each other are independently of each other H, fluorine, CN, C₁-C₂₄alkyl, C₅-C₁₂cycloalkyl, C₇-C₂₅aralkyl, C₁-C₂₄perfluoroalkyl, C₆-C₁₄perfluoroaryl, especially pentafluorophenyl, or C₁-C₂₄haloalkyl, C₆-C₁₀aryl, which can optionally be substituted by one, or more C₁-C₈alkyl, or

C₁-C₈alkoxy groups;

C₁-C₂₄alkoxy, C₁-C₂₄alkylthio, —NR²⁵R²⁶, —CONR²⁵R²⁶, or —COOR²⁷, or

two groups X⁴¹, X⁴², X⁴³, X⁴⁴, X⁴⁵, X⁴⁶, X⁴⁷, X⁴⁸, X⁴⁹, X⁵⁰, X⁵¹, X⁵², X⁵³, X⁵⁴, X⁵⁵, X⁵⁶, X⁵⁷, X⁵⁸, X⁵⁹, X⁶⁰, X⁶¹ X⁶², X⁶³, X⁶⁴, X⁶⁵, X⁶⁶ and X⁶⁷, which are neighbouring to each other, are a group

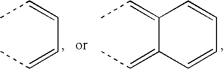

wherein preferably at least one of the substituents X⁴¹, X⁴², X⁴³, X⁴⁴, X⁴⁵, X⁴⁶, X⁴⁷, X⁴⁸, X⁴⁹, X⁵⁰, X⁵¹, X⁵², X⁵³, X⁵⁴, X⁵⁵, X⁵⁶, X⁵⁷, X⁵⁸, X⁵⁹, X⁶⁰, X⁶¹, X⁶², X⁶³, X⁶⁴, X⁶⁵, X⁶⁶ and X⁶⁷ is fluorine, —NR²⁵R²⁶, C₁-C₂₄alkyl, C₅-C₁₂cycloalkyl, C₇-C₂₅aralkyl, C₁-C₂₄perfluoroalkyl, C₆-C₁₄perfluoroaryl, especially pentafluorophenyl, or C₁-C₂₄haloalkyl, or A⁴³ or A⁵² are a group of formula

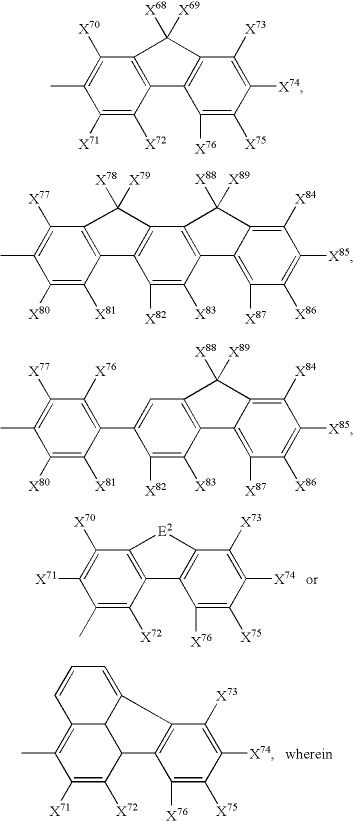

X⁶⁸, X⁶⁹, X⁷⁸, X⁷⁹, X⁸⁸ and X⁸⁹ are independently of each other C₁-C₂₄alkyl, especially C₁-C₁₂alkyl, which can be interrupted by one or two oxygen atoms,

X⁷⁰, X⁷¹, X⁷², X⁷³, X⁷⁴, X⁷⁵, X⁷⁶, X⁷⁷, X⁸⁰, X⁸¹, X⁸², X⁸³, X⁸⁴, X⁸⁵, X⁸⁶ and X⁸⁷ are independently of each other H, CN, C₁-C₂₄alkyl, C₆-C₁₀aryl, C₁-C₂₄alkoxy, C₁-C₂₄alkylthio, —NR²⁵R²⁶, —CONR²⁵R²⁶, or —COOR²⁷,

E² is —S—, —O—, or —NR^{25′}—,

A⁴¹, A⁴² and A⁴⁴ are independently of each other hydrogen, halogen, C₁-C₂₄alkyl, C₁-C₂₄perfluoroalkyl, C₆-C₁₄perfluoroaryl, especially pentafluorophenyl, C₅-C₁₂cycloalkyl, C₇-C₂₅aralkyl, C₁-C₂₄haloalkyl, C₆-C₁₈aryl, —NR²⁵R²⁶, —CONR²⁵R²⁶, or —COOR²⁷, or C₂-C₁₀heteroaryl, especially a group of formula

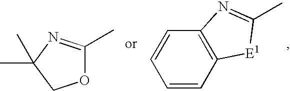

or

A⁵¹, A⁵³, A⁵⁴, A⁵⁵, A⁵⁶, A⁵⁷, A⁵⁸, A⁵⁹ and A⁶⁰ are independently of each other H, fluorine, CN, C₁-C₂₄alkyl, C₁-C₂₄alkoxy, C₁-C₂₄alkylthio, C₅-C₁₂cycloalkyl, C₇-C₂₅aralkyl, C₁-C₂₄perfluoroalkyl, C₆-C₁₄perfluoroaryl, especially pentafluorophenyl, C₁-C₂₄haloalkyl, C₆-C₁₈aryl, —NR²⁵R²⁶, —CONR²⁵R²⁶, or —COOR²⁷, or C₂-C₁₀heteroaryl, wherein E¹ is O, S, or —NR^{25′}—,

R²⁵ and R²⁶ are independently of each other H, C₆-C₁₈aryl, C₇-C₁₈aralkyl, or C₁-C₂₄alkyl, or

R²⁵ and R²⁶ together form a five or six membered ring, in particular

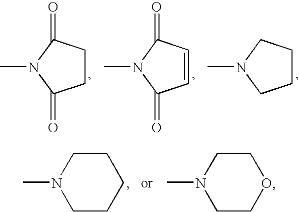

R²⁷ is C₁-C₂₄alkyl, and

Y¹ is a group of formula

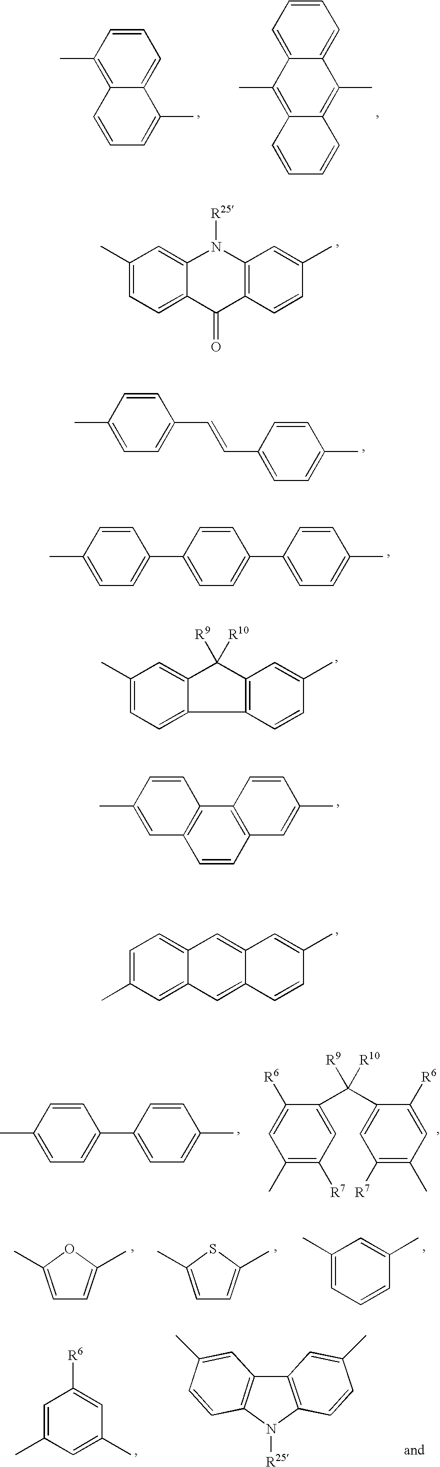

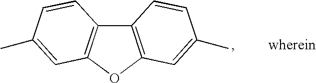

R⁶ is C₁-C₂₄alkoxy, or —O—C₇-C₂₅aralkyl, R⁷ is H, or C₁-C₂₄alkyl, R⁹ and R¹⁰ are independently of each other C₁-C₂₄alkyl, especially C₄-C₁₂alkyl, which can be interrupted by one or two oxygen atoms, and

R^{25′} is C₁-C₂₄alkyl, or C₆-C₁₀aryl.

In a particular preferred embodiment the 2H-benzotriazole compound is a compound of formula

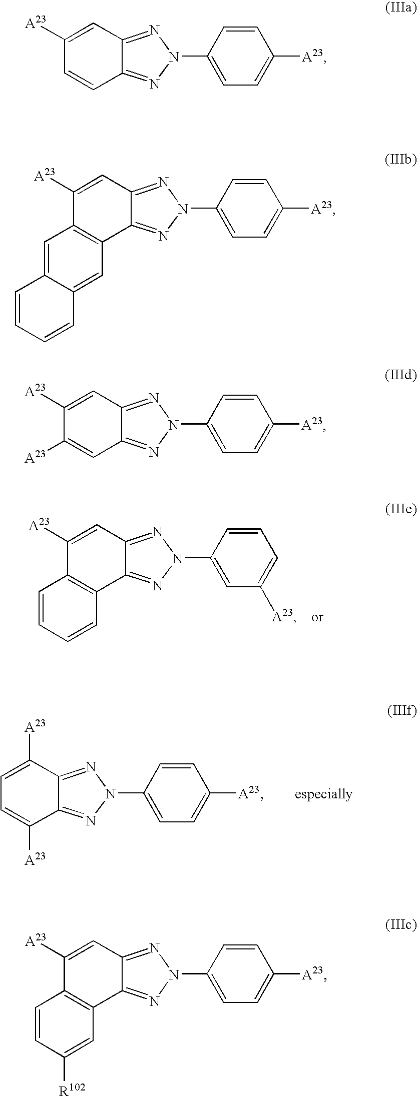

wherein R¹⁰² is C₁-C₂₄alkyl, especially C₁-C₁₂alkyl, in particular H, A²³ is a group of formula

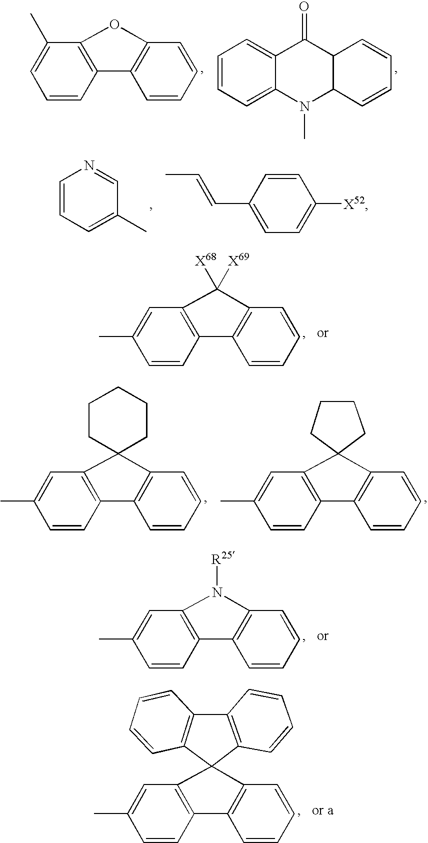

group of formula

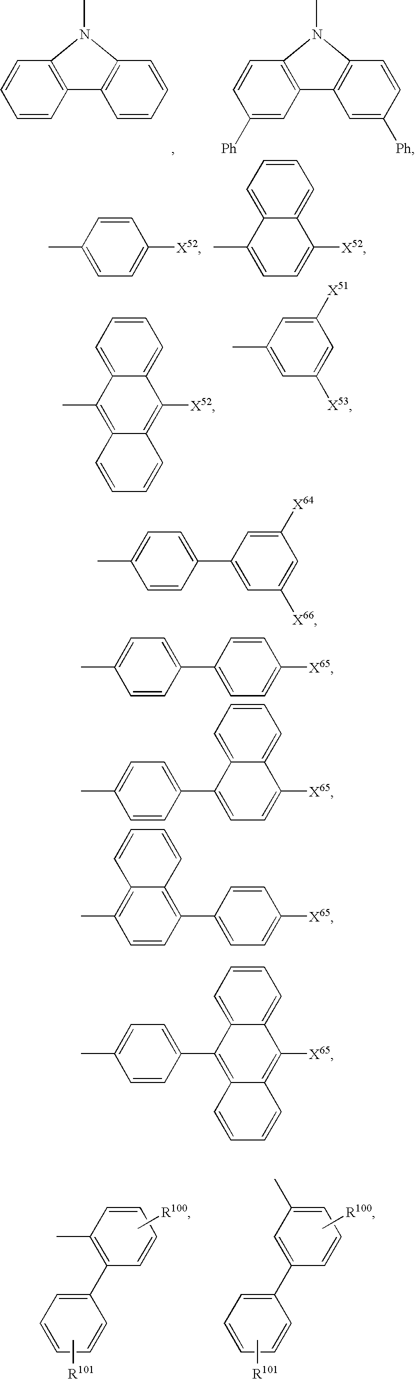

especially

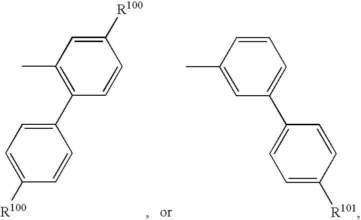

wherein R¹⁰⁰ and R¹⁰¹ are independently of each other H, C₁-C₂₄alkyl, especially C₁-C₁₂alkyl, very especially tert-butyl,

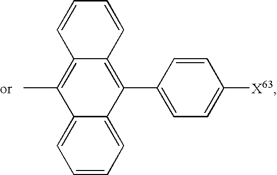

wherein X⁵¹, X⁵², X⁵³, X⁶³, X⁶⁴, X⁶⁵ and X⁶⁶ are independently of each other fluorine, C₁-C₂₄alkyl, especially C₁-C₁₂alkyl, very especially tert-butyl, C₅-C₁₂cycloalkyl, especially cyclohexyl, which can optionally be substituted by one, or two C₁-C₈alkyl groups, or 1-adamantyl, C₁-C₂₄perfluoroalkyl, especially C₁-C₁₂perfluoroalkyl, such as CF₃, C₆-C₁₄perfluoroaryl, especially pentafluorophenyl, NR²⁵R²⁶, wherein R²⁵ and R²⁶ are C₆-C₁₄aryl, especially phenyl, which can be substituted by one, or two C₁-C₂₄alkyl groups, or R²⁵ and R²⁶ together form a five or six membered heterocyclic ring, especially

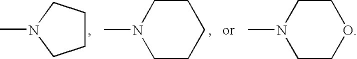

Examples of especially preferred 2H-benzotriazole compounds are shown below:

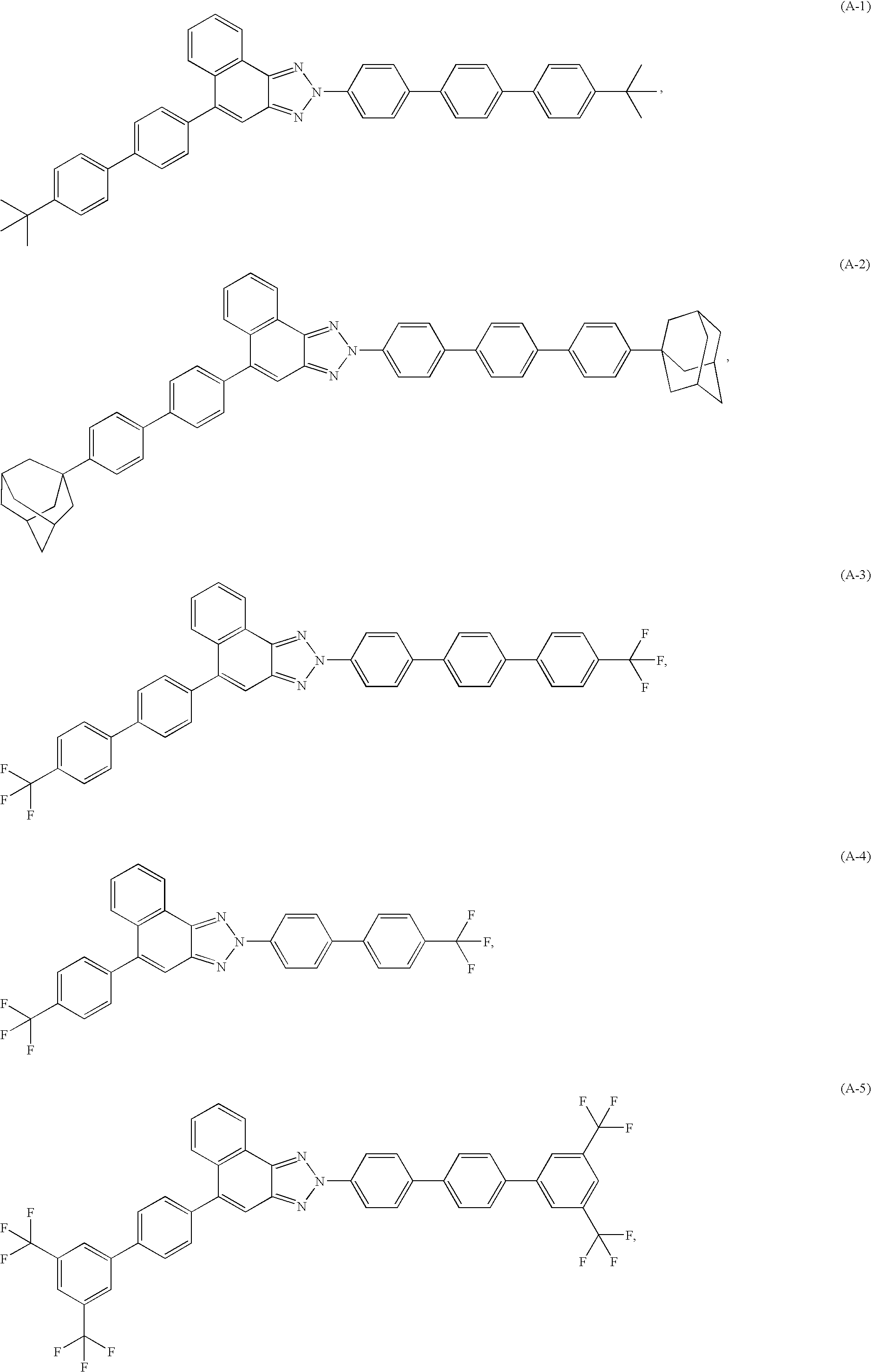

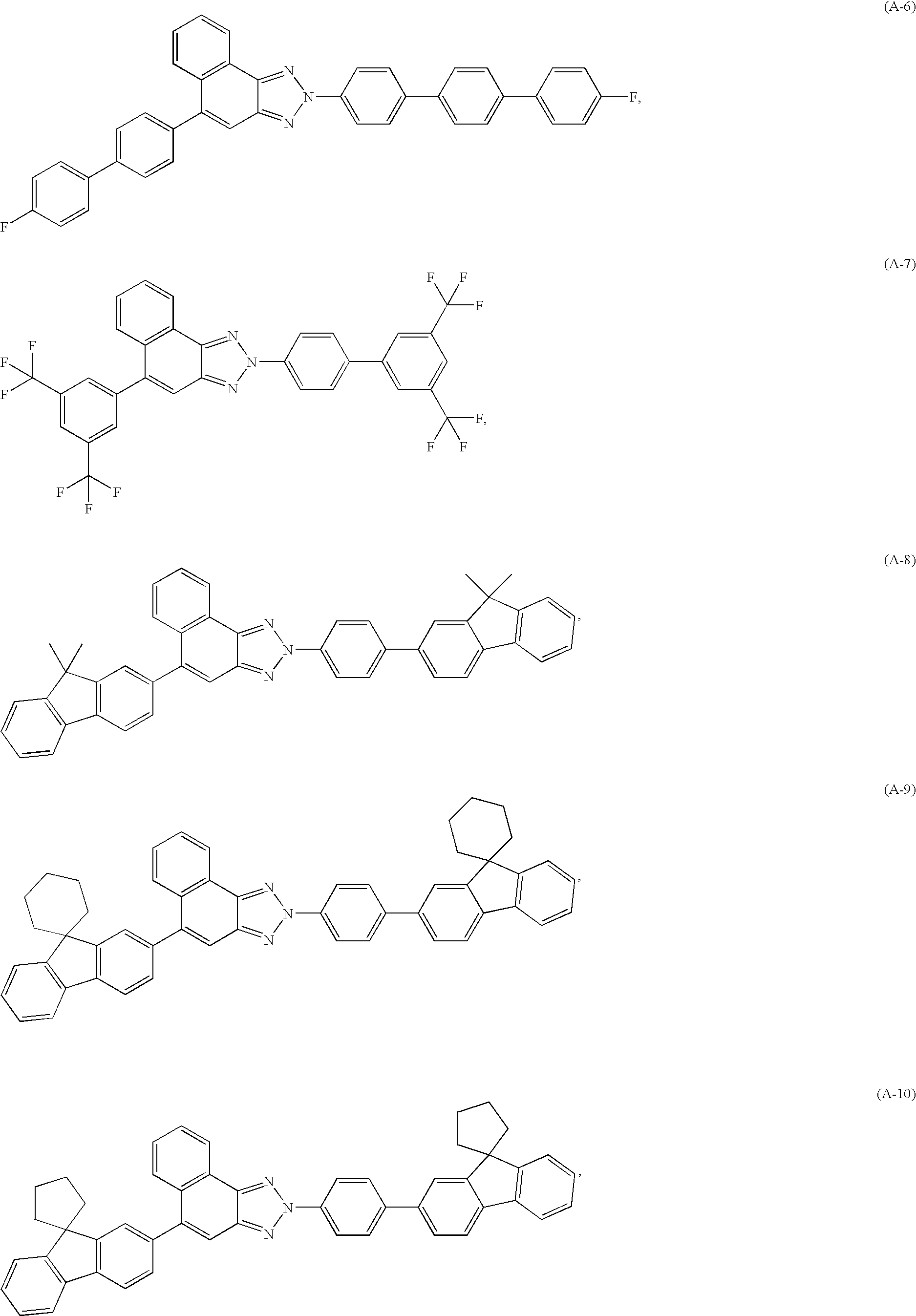

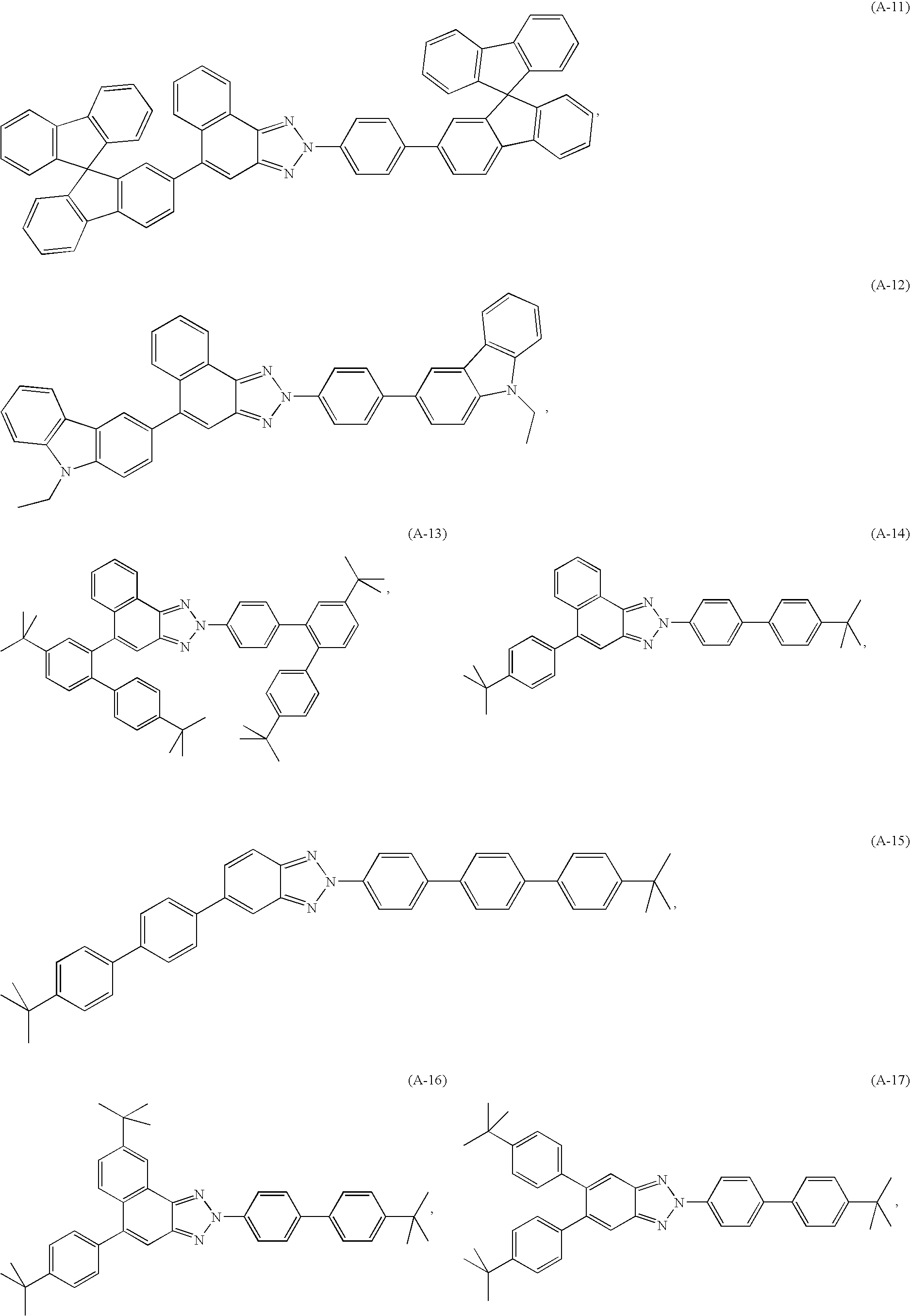

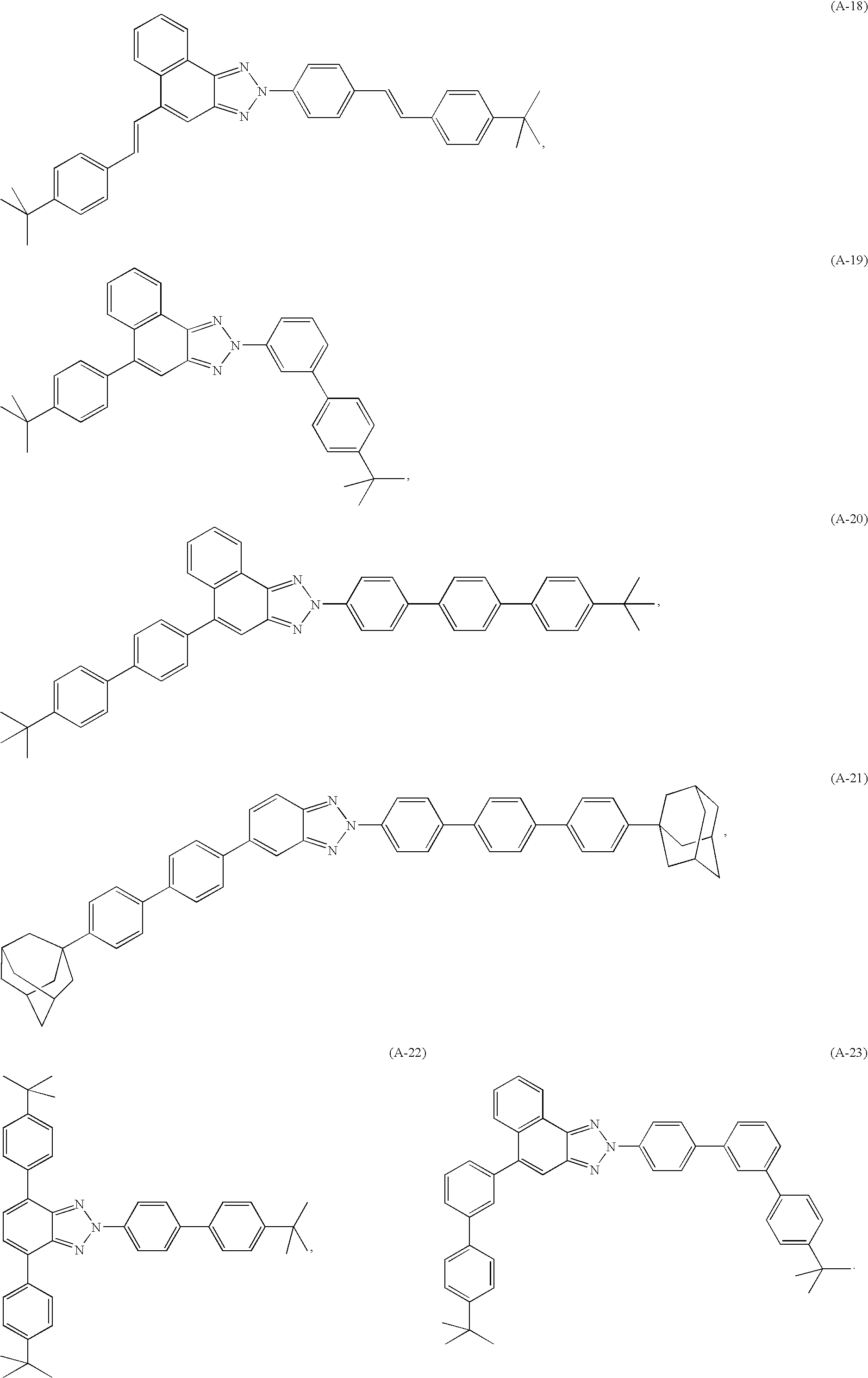

In a further preferred embodiment the present invention relates to compounds of formula

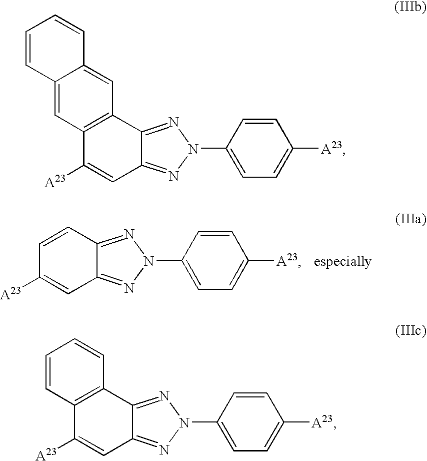

wherein one group A²³ per molecule is as defined above,
and the other group A²³ is a group of formula

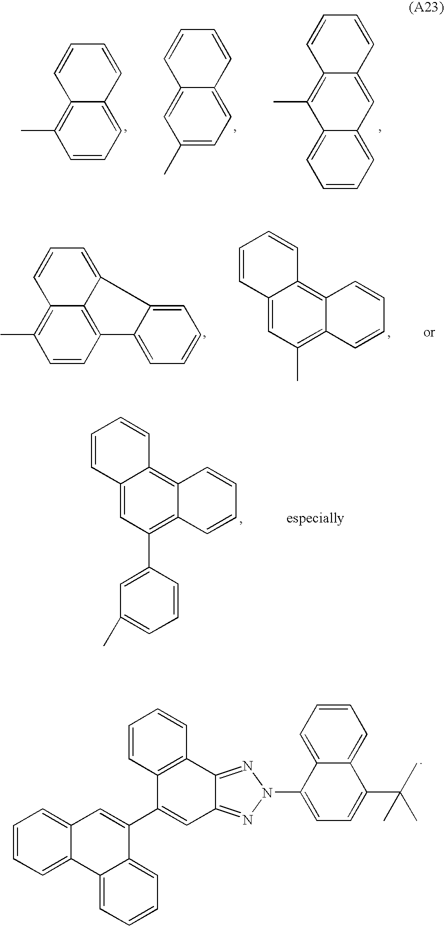

In a further preferred embodiment the present invention relates to compounds of formula

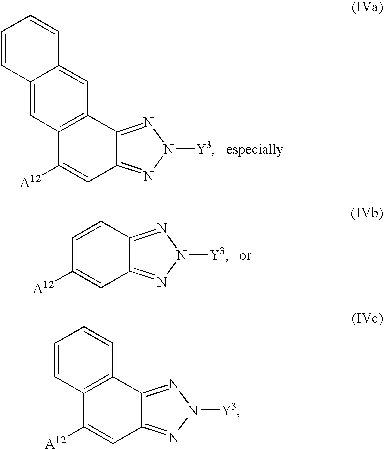

wherein

Y³ is as defined above, or is

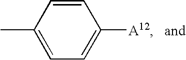

A¹² is NR²⁶R²⁸,

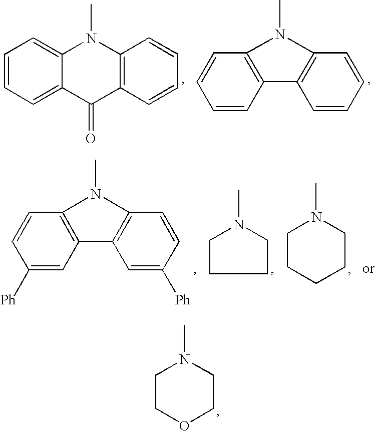

wherein R²⁵ and R²⁶ are C₆-C₁₄aryl, especially phenyl, 1-naphthyl, 2-naphthyl, which can optionally be substituted by one, or two C₁-C₈alkyl groups, or C₁-C₈alkoxy groups.

If A¹² is a group of formula

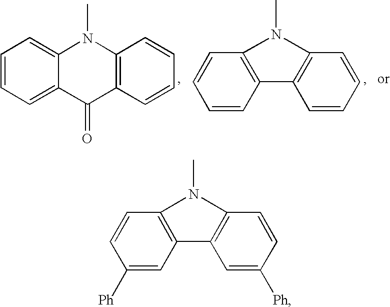

Y³ is preferably a group of formula

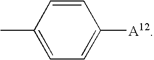

### If A¹² is a group of formula NR²⁵R²⁶,

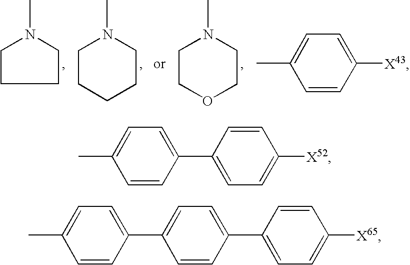

wherein X⁴³, X⁵² and X⁶⁵ are independently of each other fluorine, C₁-C₂₄alkyl, especially C₁-C₁₂alkyl, very especially tert-butyl, C₅-C₁₂cycloalkyl, especially cyclohexyl, which can optionally be substituted by one, or two C₁-C₈alkyl groups, or 1-adamantyl, C₁-C₂₄perfluoroalkyl, especially C₁-C₁₂perfluoroalkyl, such as CF₃.

In a further preferred embodiment the present invention relates to compounds of formula IVa, IVb, or IVc, wherein A¹² is

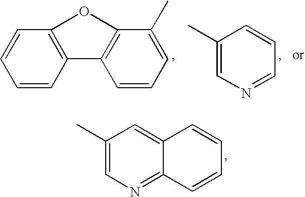

and Y³ is is

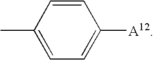

Examples of especially preferred 2H-benzotriazole compounds are shown below:

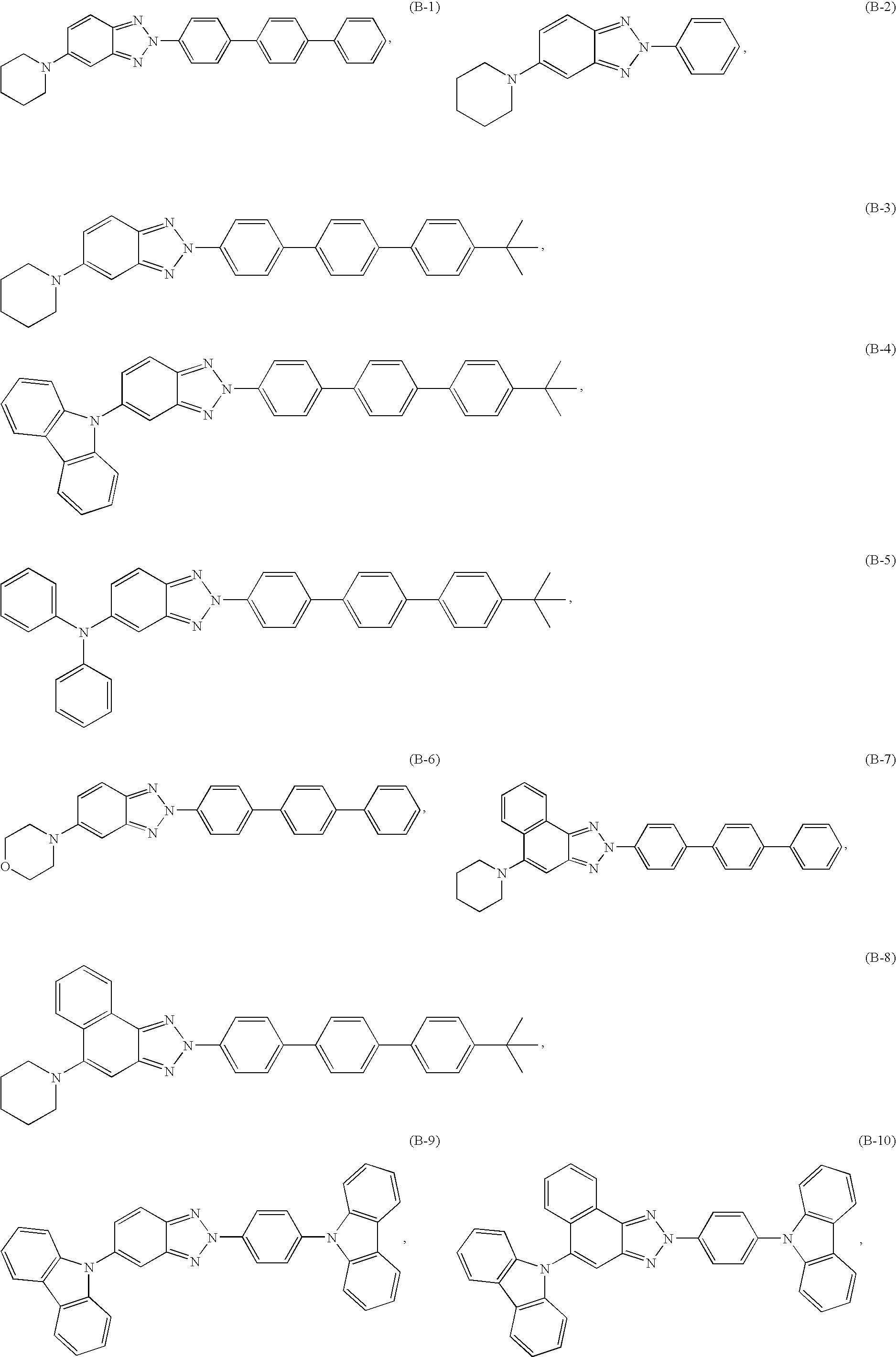

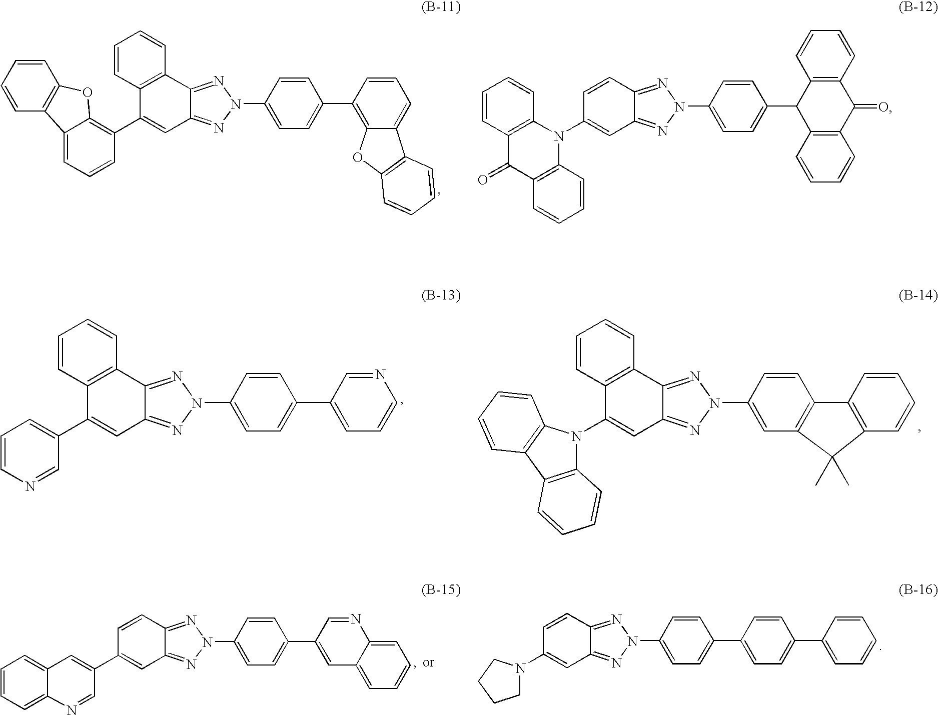

In a further preferred embodiment the present invention relates to compounds of formula

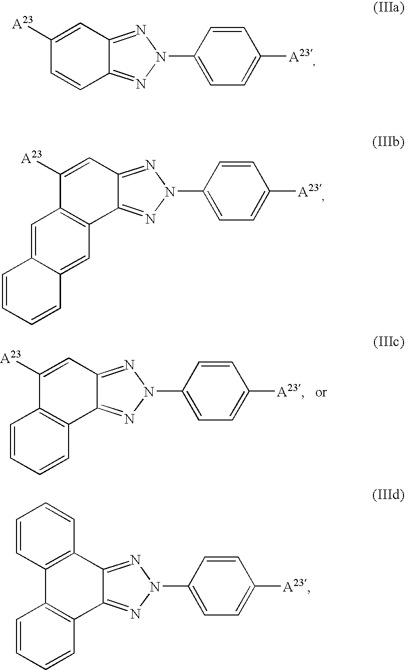

wherein A²³ and A^{23′} are independently of each other a group of formula

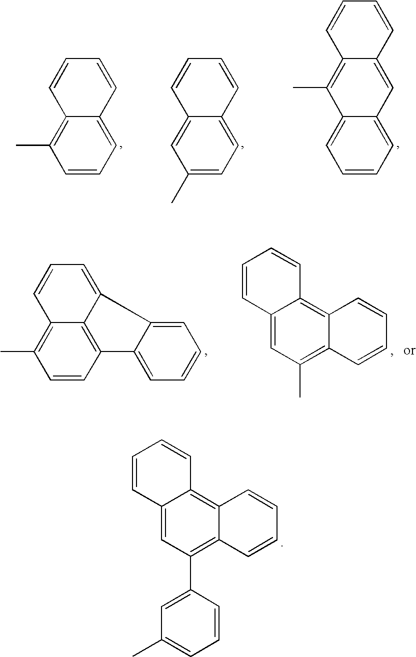

A²³ and A^{23′} have preferably the same meaning. Examples of especially preferred 2H-benzotriazole compounds are shown below:

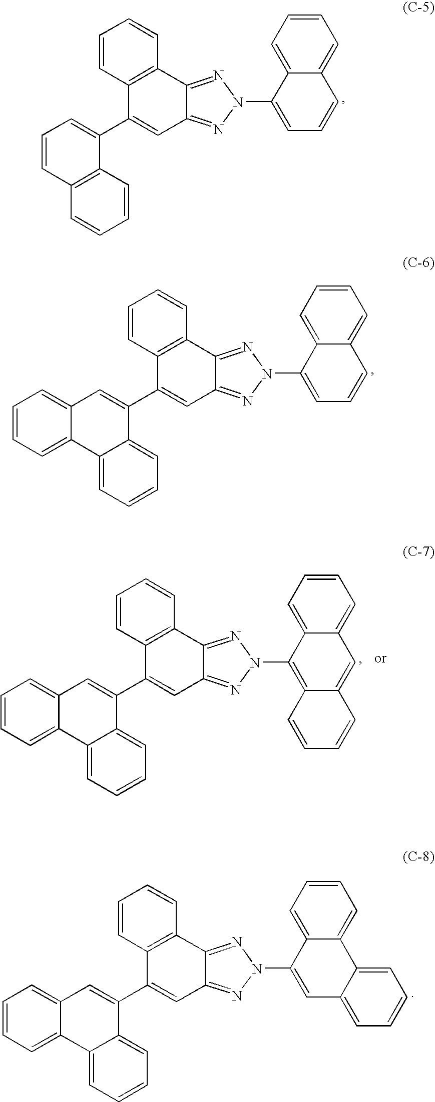

In a further preferred embodiment the present invention relates to compounds of formula

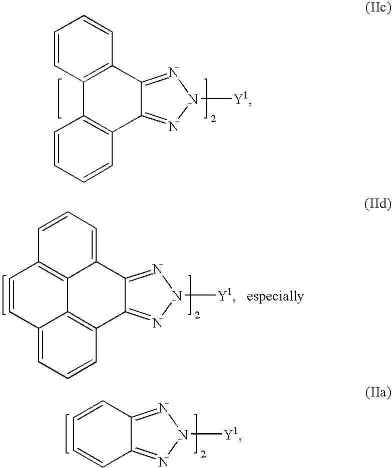

very especially

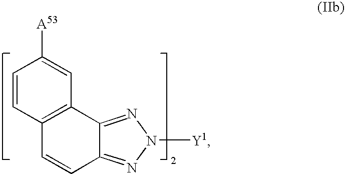

wherein A⁵³ is C₁-C₂₄alkyl, especially C₄-C₁₂alkyl, in particular H, Y¹ is a group of formula

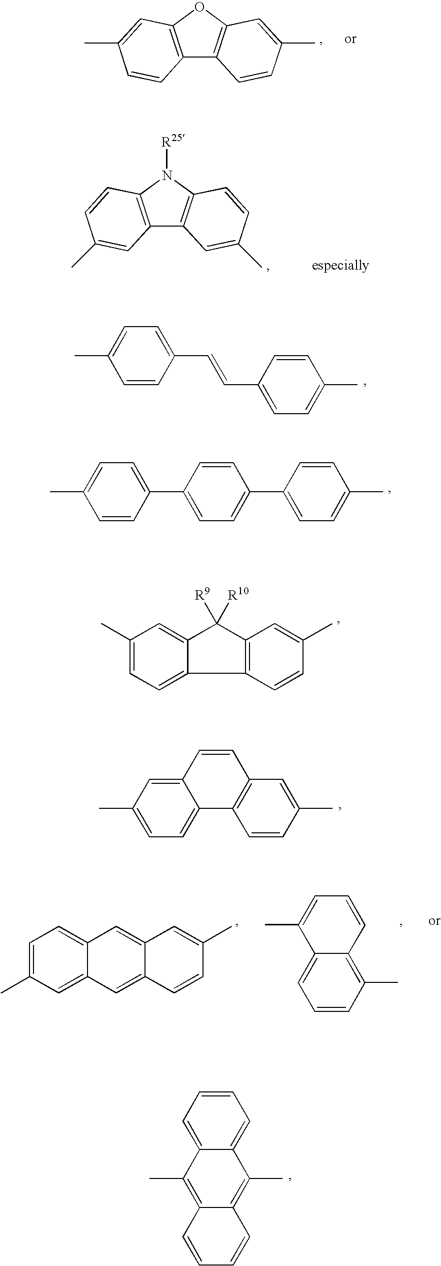

wherein R⁹ and R¹⁰ are independently of each other C₁-C₂₄alkyl, especially C₄-C₁₂alkyl, which can be interrupted by one or two oxygen atoms, and R^{25′} is C₁-C₂₄alkyl, especially C₄-C₁₂alkyl. Examples of especially preferred 2H-benzotriazole compounds are shown below:

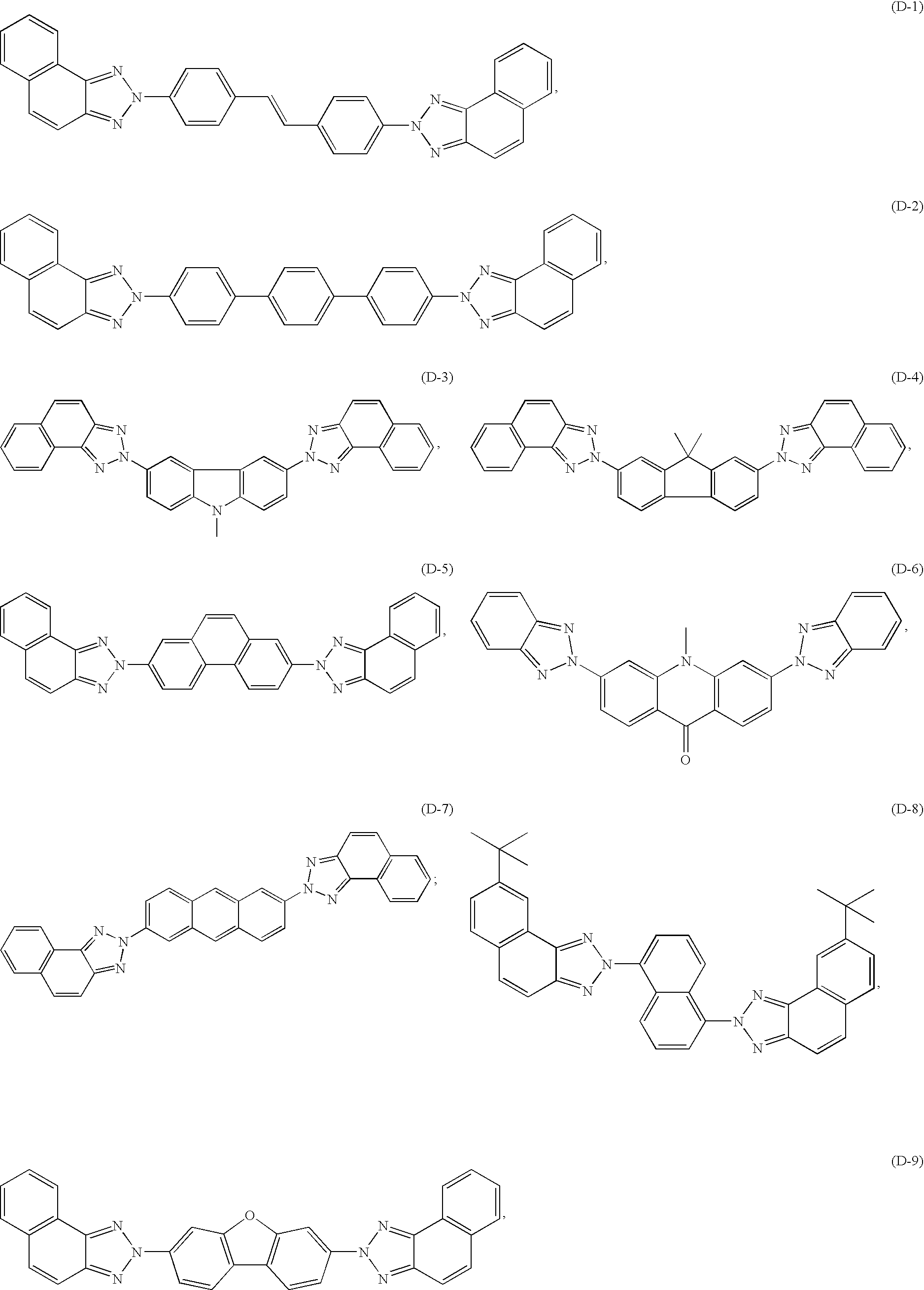

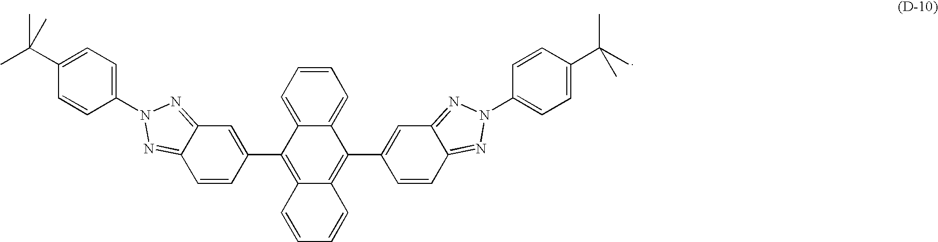

In a further preferred embodiment the present invention relates to compounds of formula Ia, Ib, Ic, or Id, especially

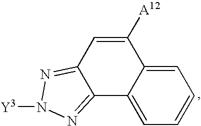

wherein A¹² is H, a group of formula

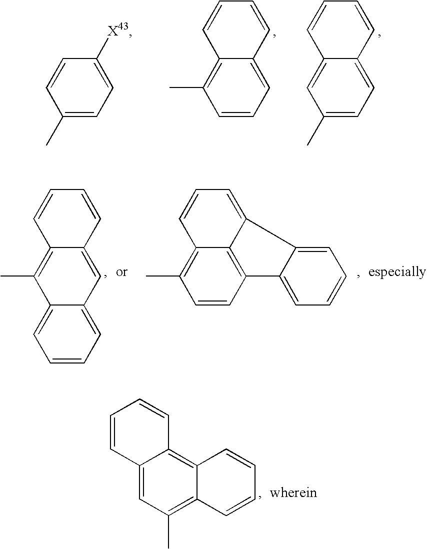

X⁴³ is C₁-C₂₄alkyl, especially C₁-C₁₂alkyl, ³ is a group of formula

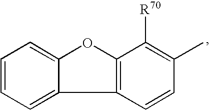

wherein R⁷⁰ is C₁-C₂₄alkyl, especially C₁-C₂₄alkoxy. Examples of especially preferred 2H-benzotriazole compounds are shown below:

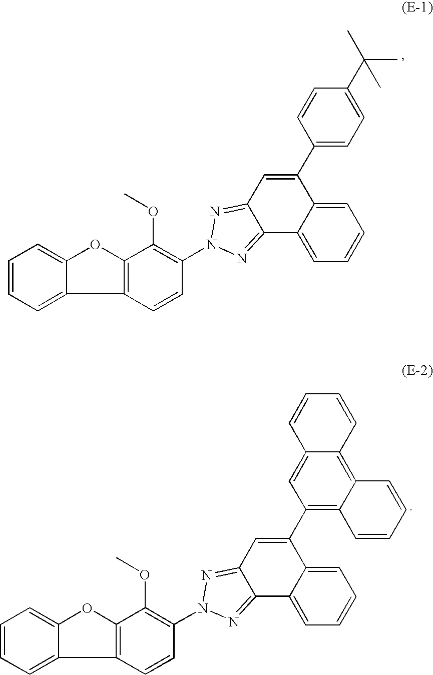

In a further preferred embodiment the present invention relates to compounds of formula Ic, especially

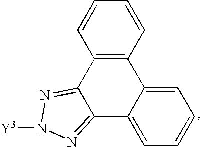

wherein Y³ is a group of formula

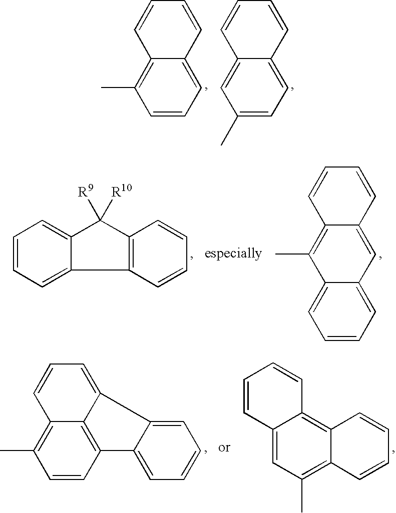

wherein R⁹ and R¹⁰ are independently of each other C₁-C₂₄alkyl, especially C₄-C₁₂alkyl, which can be interrupted by one or two oxygen atoms. Examples of especially preferred 2H-benzotriazole compounds are shown below:

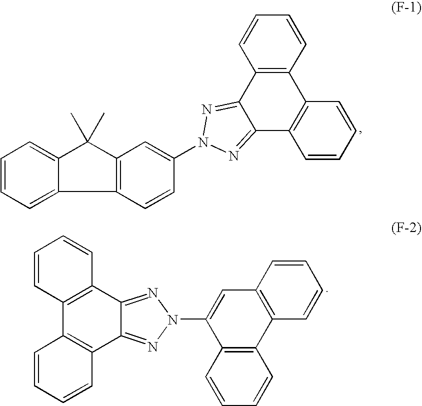

The 2H-benzotriazole compounds of formula IIIa, IIIb and IIIc, especially compounds A-1 to A-12, are preferably used as host compounds, whereas the 2H-benzotriazole compounds of formula IVa, IVb and IVc, especially compounds B-1 to B-10, as well as 2H-benzotriazole compounds of formula IIa, IIb, IIc and IId, especially compounds D-1 to D-9, are preferably used as guest compounds in the light emitting layer of EL devices.

If the 2H-benzotriazole compounds of formula IIIa, IIIb and IIIc (=III) are used as host and the 2H-benzotriazole compounds of formula IVa, IVb and IVc (=IV), or of formula IIa, IIb, IIc and IId (=II) are used as guest, the weight ratio of the the 2H-benzotriazole compound of the formula III to the 2H-benzotriazole compound of the formula IV, or II is in general 50:50 to 99.99:0.01, preferably 90:10 to 99.99:0.01, more preferably 95:5 to 99.9:0.1.

The inventive 2H-benzotriazole compounds can be synthesized according to or in analogy to methods well known in the art (see, for example, WO03/105538).

The 2H-benzotriazoles of may be prepared by any suitable process, for example, by the condensation reaction of an aromatic boronate and a bromide, commonly referred to as the “Suzuki reaction”, which is tolerant of the presence of a variety of organic functional groups as reported by N. Miyaua and A. Suzuki in Chemical Reviews, Vol. 95, pp. 457-2483 (1995).

To prepare 2H-benzotriazoles corresponding to formula (I) 2 equivalents of a bromide of formula

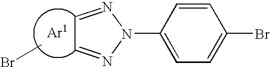

are reacted with two equivalents boronate corresponding to formula X¹¹-A²³ or a mixture thereof, wherein X¹¹ is independently in each occurrence a —B(OH)₂, —B(OY¹¹)₂ or

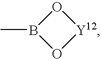

wherein Y¹¹ is independently in each occurrence a C₁-C₁₀alkyl group and Y¹² is independently in each occurrence a C₂-C₁₀alkylene group, such as —CY¹³Y¹⁴—CY⁵Y⁶—, or —CY⁷Y⁸—CY⁹Y¹⁰—CY¹⁵Y¹⁶—, wherein Y⁵, Y⁶, Y⁷, Y⁸, Y⁹, Y¹⁰, Y¹³, Y¹⁴, Y¹⁵ and Y¹⁶ are independently of each other hydrogen, or a C₁-C₁₀alkyl group, especially —C(CH₃)₂C(CH₃)₂—, or —C(CH₃)₂CH₂C(CH₃)₂—, under the catalytic action of Pd and triphenylphosphine. The reaction is typically conducted at about 70° C. to 120° C. in an aromatic hydrocarbon solvent such as toluene. Other solvents such as dimethylformamide and tetrahydrofuran can also be used alone, or in mixtures with an aromatic hydrocarbon. An aqueous base, preferably sodium carbonate or bicarbonate, is used as the HBr scavenger. Depending on the reactivities of the reactants, a reaction may take 2 to 100 hours. Organic bases, such as, for example, tetraalkylammonium hydroxide, and phase transfer catalysts, such as, for example TBAB, can promote the activity of the boron (see, for example, Leadbeater & Marco; Angew. Chem. Int. Ed., 2003, 42, 1407 and references cited therein).

Halogen is fluorine, chlorine, bromine and iodine.

C₁-C₂₄alkyl is a branched or unbranched radical such as for example methyl, ethyl, propyl, isopropyl, n-butyl, sec-butyl, isobutyl, tert-butyl, 2-ethylbutyl; n-pentyl, isopentyl, 1-methylpentyl, 1,3-dimethylbutyl, n-hexyl, 1-methylhexyl, n-heptyl, isoheptyl, 1,1,3,3-tetramethylbutyl, 1-methylheptyl, 3-methylheptyl, n-octyl, 2-ethylhexyl, 1,1,3-trimethylhexyl, 1,1,3,3-tetramethylpentyl, nonyl, decyl, undecyl, 1-methylundecyl, dodecyl, 1,1,3,3,5,5-hexamethylhexyl, tridecyl, tetradecyl, pentadecyl, hexadecyl, heptadecyl, octadecyl, icosyl or docosyl.

C₁-C₂₄perfluoroalkyl is a branched or unbranched radical such as for example —CF₃, —CF₂CF₃, —CF₂CF₂CF₃, —CF(CF₃)₂, —(CF₂)₃CF₃, and —C(CF₃)₃.

C₁-C₂₄alkoxy radicals are straight-chain or branched alkoxy radicals, e.g. methoxy, ethoxy, n-propoxy, isopropoxy, n-butoxy, sec-butoxy, tert-butoxy, amyloxy, isoamyloxy or tert-amyloxy, heptyloxy, octyloxy, isooctyloxy, nonyloxy, decyloxy, undecyloxy, dodecyloxy, tetradecyloxy, pentadecyloxy, hexadecyloxy, heptadecyloxy and octadecyloxy.

C₂-C₂₄alkenyl radicals are straight-chain or branched alkenyl radicals, such as e.g. vinyl, allyl, methallyl, isopropenyl, 2-butenyl, 3-butenyl, isobutenyl, n-penta-2,4-dienyl, 3-methyl-but-2-enyl, n-oct-2-enyl, n-dodec-2-enyl, isododecenyl, n-dodec-2-enyl or n-octadec-4-enyl.

C₂-₂₄alkynyl is straight-chain or branched and preferably C_{2 8}alkynyl, which may be unsubstituted or substituted, such as, for example, ethynyl, 1-propyn-3-yl, 1butyn-4-yl, 1-pentyn-5-yl, 2-methyl-3-butyn-2-yl, 1,4-pentadiyn-3-yl, 1,3-pentadiyn-5-yl, 1-hexyn-6-yl, cis-3-methyl-2-penten-4-yn-1-yl, trans-3-methyl-2-penten4-yn-1-yl, 1,3-hexadiyn-5-yl, 1-octyn-8-yl, 1-nonyn-9-yl, 1-decyn-10-yl, or 1-tetracosyn-24-yl.

C₄-C₁₈cycloalkyl, especially C₅-C₁₂cycloalkyl, is preferably C₅-C₁₂cycloalkyl or said cycloalkyl substituted by one to three C₁-C₄alkyl groups, such as, for example, cyclopentyl, methylcyclopentyl, dimethylcyclopentyl, cyclohexyl, methylcyclohexyl, dimethylcyclohexyl, trimethylcyclohexyl, tert-butylcyclohexyl, cycloheptyl, cyclooctyl, cyclononyl, cyclodecyl, cyclododecyl, 1-adamantyl, or 2-adamantyl. Cyclohexyl, 1-adamantyl and cyclopentyl are most preferred.

Examples of C₄-C₁₈cycloalkyl, which is interrupted by S, O, or NR²⁵, are piperidyl, piperazinyl and morpholinyl.

Aryl is usually C₆-C₃₀aryl, preferably C₆-C₂₄aryl, which optionally can be substituted, such as, for example, phenyl, 4-methylphenyl, 4-methoxyphenyl, naphthyl, biphenylyl, 2-fluorenyl, phenanthryl, anthryl, tetracyl, pentacyl, hexacyl, terphenylyl or quadphenylyl; or phenyl substituted by one to three C₁-C₄alkyl groups, for example o-, m- or p-methylphenyl, 2,3-dimethylphenyl, 2,4-dimethylphenyl, 2,5-dimethylphenyl, 2,6-dimethylphenyl, 3,4-dimethylphenyl, 3,5-dimethylphenyl, 2-methyl-6-ethylphenyl, 4-tert-butylphenyl, 2-ethylphenyl or 2,6-diethylphenyl.

C₇-C₂₄aralkyl radicals are preferably C₇-C₁₅aralkyl radicals, which may be substituted, such as, for example, benzyl, 2-benzyl-2-propyl, β-phenethyl, α-methylbenzyl, α,α-dimethylbenzyl, ω-phenyl-butyl, ω-phenyl-octyl, ω-phenyl-dodecyl; or phenyl-C₁-C₄alkyl substituted on the phenyl ring by one to three C₁-C₄alkyl groups, such as, for example, 2-methylbenzyl, 3-methylbenzyl, 4-methylbenzyl, 2,4-dimethylbenzyl, 2,6-dimethylbenzyl or 4-tert-butylbenzyl. or 3-methyl-5-(1′,1 ′,3′,3′-tetramethyl-butyl)-benzyl.

Heteroaryl is typically C₂-C₂₆heteroaryl, i.e. a ring with five to seven ring atoms or a condensed ring system, wherein nitrogen, oxygen or sulfur are the possible hetero atoms, and is typically an unsaturated heterocyclic radical with five to 30 atoms having at least six conjugated π-electrons such as thienyl, benzo[b]thienyl, dibenzo[b,d]thienyl, thianthrenyl, furyl, furfuryl, 2H-pyranyl, benzofuranyl, isobenzofuranyl, dibenzofuranyl, phenoxythienyl, pyrrolyl, imidazolyl, pyrazolyl, pyridyl, bipyridyl, triazinyl, pyrimidinyl, pyrazinyl, pyridazinyl, indolizinyl, isoindolyl, indolyl, indazolyl, purinyl, quinolizinyl, chinolyl, isochinolyl, phthalazinyl, naphthyridinyl, chinoxalinyl, chinazolinyl, cinnolinyl, pteridinyl, carbazolyl, carbolinyl, benzotriazolyl, benzoxazolyl, phenanthridinyl, acridinyl, perimidinyl, phenanthrolinyl, phenazinyl, isothiazolyl, phenothiazinyl, isoxazolyl, furazanyl or phenoxazinyl, which can be unsubstituted or substituted.

C₆-C₁₈cycloalkoxy is, for example, cyclopentyloxy, cyclohexyloxy, cycloheptyloxy or cyclooctyloxy, or said cycloalkoxy substituted by one to three C₁-C₄alkyl, for example, methylcyclopentyloxy, dimethylcyclopentyloxy, methylcyclohexyloxy, dimethylcyclohexyloxy, trimethylcyclohexyloxy, or tert-butylcyclohexyloxy.

C₆-C₂₄aryloxy is typically phenoxy or phenoxy substituted by one to three C₁-C₄alkyl groups, such as, for example o-, m- or p-methylphenoxy, 2,3-dimethylphenoxy, 2,4-dimethylphenoxy, 2,5-dimethylphenoxy, 2,6-dimethylphenoxy, 3,4-dimethylphenoxy, 3,5-dimethylphenoxy, 2-methyl-6-ethylphenoxy, 4-tert-butylphenoxy, 2-ethylphenoxy or 2,6-diethylphenoxy.

C₆-C₂₄aralkoxy is typically phenyl-C₁-C₉alkoxy, such as, for example, benzyloxy, α-methylbenzyloxy, α,α-dimethylbenzyloxy or 2-phenylethoxy.

C₁-C₂₄alkylthio radicals are straight-chain or branched alkylthio radicals, such as e.g. methylthio, ethylthio, propylthio, isopropylthio, n-butylthio, isobutylthio, pentylthio, isopentylthio, hexylthio, heptylthio, octylthio, decylthio, tetradecylthio, hexadecylthio or octadecylthio.

Examples of a five or six membered ring formed by R⁹ and R¹⁰ and R²⁵ and R²⁶, respectively are heterocycloalkanes or heterocycloalkenes having from 3 to 5 carbon atoms which can have one additional hetero atom selected from nitrogen, oxygen and sulfur, for example

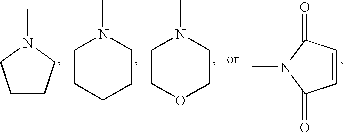

which can be part of a bicyclic system, for example

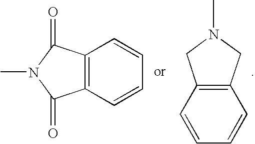

Possible substituents of the above-mentioned groups are C₁-C₈alkyl, a hydroxyl group, a mercapto group, C₁-C₈alkoxy, C₁-C₈alkylthio, halogen, halo-C₁-C₈alkyl, a cyano group, an aldehyde group, a ketone group, a carboxyl group, an ester group, a carbamoyl group, an amino group, a nitro group or a silyl group.

The term “haloalkyl” means groups given by partially or wholly substituting the above-mentioned alkyl group with halogen, such as trifluoromethyl etc. The “aldehyde group, ketone group, ester group, carbamoyl group and amino group” include those substituted by an C₁-C₂₄alkyl group, a C₄-C₁₈cycloalkyl group, an C₆-C₃₀aryl group, an C₇-C₂₄aralkyl group or a heterocyclic group, wherein the alkyl group, the cycloalkyl group, the aryl group, the aralkyl group and the heterocyclic group may be unsubstituted or substituted. The term “silyl group” means a group of formula —SiR¹⁰⁵R¹⁰⁶R¹⁰⁷, wherein R¹⁰⁵, R¹⁰⁶ and R¹⁰⁷ are independently of each other a C₁-C₈alkyl group, in particular a C₁-C₄alkyl group, a C₆-C₂₄aryl group or a C₇-C₁₂aralkyl group, such as a trimethylsilyl group.

If a substituent, such as, for example R⁶ and R⁷, occurs more than one time in a group, it can be different in each occurrence.

As described above, the aforementioned radicals may be substituted by E and/or, if desired, interrupted by D. Interruptions are of course possible only in the case of radicals containing at least 2 carbon atoms connected to one another by single bonds; C₆-C₁₈aryl is not interrupted; interrupted arylalkyl or alkylaryl contains the unit D in the alkyl moiety. C₁-C₂₄alkyl substituted by one or more E and/or interrupted by one or more units D is, for example, (CH₂CH₂O)₁₋₉—R^{x}, where R^{x} is H or C₁-C₁₀alkyl or C₂-C₁₀alkanoyl (e.g. CO—CH(C₂H₅)C₄H₉), CH₂—CH(OR^{y′})—CH₂—O—R^{y}, where R^{y} is C₁-C₂₄alkyl, C₅-C₁₂cycloalkyl, phenyl, C₇-C₁₅phenylalkyl, and R^{y′} embraces the same definitions as R^{y} or is H; C₁-C₈alkylene-COO—R^{z}, e.g. CH₂COOR^{z}, CH(CH₃)COOR^{z}, C(CH₃)₂COOR^{z}, where R^{z} is H, C₁-C₂₄alkyl, (CH₂CH₂O)₁₋₉—R^{x}, and R^{x} embraces the definitions indicated above;

CH₂CH₂—O—CO—CH═CH₂; CH₂CH(OH)CH₂—O—CO—C(CH₃)═CH₂.

The blue-emitting 2H-benzotriazoles of this invention emit light below about 520 nm, for example between about 380 nm and about 520 nm. For example, the blue-emitting 2H-benzotriazoles of this invention have a NTSC coordinate of about (0.14, 0.08), where the first coordinate is between about 0.12 and about 0.16, and the second coordinate is between about 0.05 and about 0.10.

The present compounds of formula I, II, III, or IV may also function as other than a blue-emitting organic compound, for example they may also function as a hole-injecting, hole-transporting, and electron-injecting or an electron-transporting material. The organic EL device of the present invention has significant industrial values since it can be adapted for a flat panel display of an on-wall television set, a flat light-emitting device, a light source for a copying machine or a printer, a light source for a liquid crystal display or counter, a display signboard and a signal light.

The material of the present invention can be used in the fields of an organic EL device, an electrophotographic photoreceptor, a photoelectric converter, a solar cell, and an image sensor.

To obtain organic layers of this invention with the proper T_{g}, or glass transition temperature, it is advantageous that the present 2H-benzotriazoles have a melting point greater than about 150° C., for example greater than about 200° C., for example greater than about 250° C., for instance greater than about 300° C.

The electroluminescent devices of the present invention are otherwise designed as is known in the art, for example as described in U.S. Pat. Nos. 5,518,824, 6,280,859, 5,629,389, 5,486,406, 5,104,740 and 5,116,708, the relevant disclosures of which are hereby incorporated by reference.

The present invention relates to an electroluminescent device having the 2H-benzotriazoles of of formula I between an anode and a cathode and emitting light by the action of electrical energy.

Typical constitutions of latest organic electroluminescent devices are:
(i) an anode/a hole transporting layer/an electron transporting layer/a cathode, in which 2H-benzotriazoles of of formula I are used either as positive-hole transport compound, which is exploited to form the light emitting and hole transporting layers, or as electron transport compound, which can be exploited to form the light-emitting and electron transporting layers,
(ii) an anode/a hole transporting layer/a light-emitting layer/an electron transporting layer/a cathode, in which the 2H-benzotriazoles of of formula I form the light-emitting layer regardless of whether they exhibit positive-hole or electron transport properties in this constitution,
(iii) an anode/a hole injection layer /a hole transporting layer/a light-emitting layer/an electron transporting layer/a cathode,
(iv) an anode/a hole transporting layer/a light-emitting layer/a positive hole inhibiting layer/an electron transporting layer/a cathode,
(v) an anode/a hole injection layer/a hole transporting layer/a light-emitting layer/a positive hole inhibiting layer/an electron transporting layer/a cathode,
(vi) an anode/a light-emitting layer/an electron transporting layer/a cathode,
(vii) an anode/a light-emitting layer/a positive hole inhibiting layer/an electron transporting layer/a cathode,
(viii) a mono-layer containing a light emitting material alone or a combination of a light emitting material and any of the materials of the hole transporting layer, the hole-blocking layer and/or the electron transporting layer, and
(ix) a multi-layered structure described in (ii) to (vii), wherein a light emitting layer is the mono-layer defined in (viii).

The 2H-benzotriazoles of of formula I can, in principal be used for any organic layer, such as, for example, hole transporting layer, light emitting layer, or electron transporting layer, but are preferably used as the light emitting material in the light emitting layer, optionally as a host or guest component.

The light emitting compounds of this invention exhibit intense fluorescence in the solid state and have excellent electric-field-applied light emission characteristics. Further, the light emitting compounds of this invention are excellent in the injection of holes from a metal electrode and the transportation of holes; as well as being excellent in the injection of electrons from a metal electrode and the transportation of electrons. They are effectively used as light emitting materials and may be used in combination with other hole transporting materials, other electron transporting materials or other dopants.

The 2H-benzotriazoles of the present invention form uniform thin films. The light emitting layers may therefore be formed of the present 2H-benzotriazoles alone. Alternatively, the light-emitting layer may contain a known light-emitting material, a known dopant, a known hole-injecting material or a known electron-injecting material as required. In the organic EL device, a decrease in the brightness and life caused by quenching can be prevented by forming it as a multi-layered structure. The light-emitting material, a dopant, a hole-injecting material and an electron-injecting material may be used in combination as required. Further, a dopant can improve the light emission brightness and the light emission efficiency, and can attain red, green or blue light emission. Further, each of the hole-injecting zone, the light-emitting layer and the electron-injecting zone may have the layer structure of at least two layers. In the hole-injecting zone in this case, a layer to which holes are injected from an electrode is called “hole-injecting layer”, and a layer which receives holes from the hole-injecting layer and transport the holes to a light-emitting layer is called “hole-transporting layer”. In the electron-injecting zone, a layer to which electrons are injected from an electrode is called “electron-injecting layer”, and a layer which receives electrons from the electron-injecting layer and transports the electrons to a light-emitting layer is called “electron-transporting layer”. These layers are selected and used depending upon factors such as the energy level and heat resistance of materials and adhesion to an organic layer or metal electrode.

The light-emitting material or the dopant which may be used in the light-emitting layer together with the 2H-benzotriazoles of the present invention includes for example anthracene, naphthalene, phenanthrene, pyrene, tetracene, coronene, chrysene, fluorescein, perylene, phthaloperylene, naphthaloperylene, perinone, phthaoperinone, naphthaloperinone, diphenylbutadiene, tetraphenylbutadiene, coumarine, oxadiazole, aldazine, bisbenzoxazoline, bisstyryl, pyrazine, cyclopentadiene, quinoline metal complex, aminoquinoline metal complex, benzoquinoline metal complex, imine, diphenylethylene, vinyl anthracene, diaminocarbazole, pyran, thiopyran, polymethine, merocyanine, an imidazole-chelated oxynoid compound, quinacridone, rubrene, and fluorescent dyestuffs for a dyestuff laser or for brightening.

The 2H-benzotriazoles of the present invention and the above compound or compounds that can be used in a light-emitting layer may be used in any mixing ratio for forming a light-emitting layer. That is, 2H-benzotriazoles of the present invention may provide a main component for forming a light-emitting layer, or they may be a doping material in another main material, depending upon a combination of the above compounds with the present 2H-benzotriazoles of the present invention. Good results are, for example, achieved, when DPVBI (4,4′-bis-(2,2-diphenyl-1-vinyl)biphenyl) is used as host and compounds A-1 to A-12 are used as guest.

Thin film type electroluminescent devices usually consist essentially of a pair of electrodes and at least one charge transporting layer in between. Usually two charge transporting layers, a hole transporting layer (next to the anode) and an electron transporting layer (next to the cathode) are present. Either one of them contains—depending on its properties as hole-transporting or electron-transporting material—an inorganic or organic fluorescence substance as light-emitting material. It is also common, that a light-emitting material is used as an additional layer between the hole-transporting and the electron-transporting layer. In the above mentioned device structure, a hole injection layer can be constructed between an anode and a hole transporting layer and/or a positive hole inhibiting layer can be constructed between a light emitting layer and an electron transporting layer to maximise hole and electron population in the light emitting layer, reaching large efficiency in charge recombination and intensive light emission.

The devices can be prepared in several ways. Usually, vacuum evaporation is used for the preparation. Preferably, the organic layers are laminated in the above order on a commercially available indium-tin-oxide (“ITO”) glass substrate held at room temperature, which works as the anode in the above constitutions. The membrane thickness is preferably in the range of 1 to 10,000 nm, more preferably 1 to 5,000 nm, more preferably 1 to 1,000 nm, more preferably 1 to 500 nm. The cathode metal, such as a Mg/Ag alloy, a binary Li—Al or LiF—Al system with an thickness in the range of 50-200 nm is laminated on the top of the organic layers. The vacuum during the deposition is preferably less than 0.1333 Pa (1×10⁻³ Torr), more preferably less than 1.333×10⁻³ Pa (1×10⁻⁵ Torr), more preferably less than 1.333×10⁻⁴ Pa (1×10⁻⁶ Torr).

As anode usual anode materials which possess high work function such as metals like gold, silver, copper, aluminum, indium, iron, zinc, tin, chromium, titanium, vanadium, cobalt, nickel, lead, manganese, tungsten and the like, metallic alloys such as magnesium/copper, magnesium/silver, magnesium/aluminum, aluminum/indium and the like, semiconductors such as Si, Ge, GaAs and the like, metallic oxides such as indium-tin-oxide (“ITO”), ZnO and the like, metallic compounds such as Cul and the like, and furthermore, electroconducting polymers, such as polyacetylene, polyaniline, polythiophene, polypyrrole, polyparaphenylene and the like, preferably ITO, most preferably ITO on glass as substrate can be used. Of these electrode materials, metals, metallic alloys, metallic oxides and metallic compounds can be transformed into electrodes, for example, by means of the sputtering method. In the case of using a metal or a metallic alloy as a material for an electrode, the electrode can be formed also by the vacuum deposition method. In the case of using a metal or a metallic alloy as a material forming an electrode, the electrode can be formed, furthermore, by the chemical plating method (see for example, Handbook of Electrochemistry, pp 383-387, Mazuren, 1985). In the case of using an electroconducting polymer, an electrode can be made by forming it into a film by means of anodic oxidation polymerization method onto a substrate which is previously provided with an electroconducting coating. The thickness of an electrode to be formed on a substrate is not limited to a particular value, but, when the substrate is used as a light emitting plane, the thickness of the electrode is preferably within the range of from 1 nm to 300 nm, more preferably, within the range of from 5 to 200 nm so as to ensure transparency.

In a preferred embodiment ITO is used on a substrate having an ITO film thickness in the range of from 10 nm (100 Å) to 1μ (10000 Å), preferably from 20 nm (200 Å) to 500 nm (5000 Å). Generally, the sheet resistance of the ITO film is chosen in the range of not more than 100 Ω/cm², preferably not more than 50 Ω/cm².

Such anodes are commercially available from Japanese manufacturers, such as Geomatech Co. Ltd., Sanyo Vacuum Co. Ltd., Nippon Sheet Glass Co. Ltd.

As substrate either an electronconducting or electrically insulating material can be used. In case of using an electroconducting substrate, a light emitting layer or a positive hole transporting layer is directly formed thereupon, while in case of using an electrically insulating substrate, an electrode is firstly formed thereupon and then a light emitting layer or a positive hole transporting layer is superposed.

The substrate may be either transparent, semi-transparent or opaque. However, in case of using a substrate as an indicating plane, the substrate must be transparent or semi-transparent.

Transparent electrically insulating substrates are, for example, inorganic compounds such as glass, quartz and the like, organic polymeric compounds such as polyethylene, polypropylene, polymethylmethacrylate, polyacrylonitrile, polyester, polycarbonate, polyvinylchloride, polyvinylalcohol, polyvinylacetate and the like. Each of these substrates can be transformed into a transparent electroconducting substrate by providing it with an electrode according to one of the methods described above.

Examples of semi-transparent electrically insulating substrates are inorganic compounds such as alumina, YSZ (yttrium stabilized zirconia) and the like, organic polymeric compounds such as polyethylene, polypropylene, polystyrene, epoxy resins and the like. Each of these substrates can be transformed into a semi-transparent electroconducting substrate by providing it with an electrode according to one of the abovementioned methods.

Examples of opaque electroconducting substrates are metals such as aluminum, indium, iron, nickel, zinc, tin, chromium, titanium, copper, silver, gold, platinum and the like, various electroplated metals, metallic alloys such as bronze, stainless steel and the like, semiconductors such as Si, Ge, GaAs, and the like, electroconducting polymers such as polyaniline, polythiophene, polypyrrole, polyacetylene, polyparaphenylene and the like.

A substrate can be obtained by forming one of the above listed substrate materials to a desired dimension. It is preferred that the substrate has a smooth surface. Even, if it has a rough surface, it will not cause any problem for practical use, provided that it has round unevenness having a curvature of not less than 20 μm. As for the thickness of the substrate, there is no restriction as far as it ensures sufficient mechanical strength.

As cathode usual cathode materials which possess low work function such as alkali metals, earth alkaline metals, group 13 elements, silver, and copper as well as alloys or mixtures thereof such as sodium, lithium, potassium, calcium, lithium fluoride (LiF), sodium-potassium alloy, magnesium, magnesium-silver alloy, magnesium-copper alloy, magnesium-aluminum alloy, magnesium-indium alloy, aluminum, aluminum-aluminum oxide alloy, aluminum-lithium alloy, indium, calcium, and materials exemplified in EP-A 499,011 such as electroconducting polymers e.g. polypyrrole, polythiophene, polyaniline, polyacetylene etc., preferably Mg/Ag alloys, LiF—Al or Li—Al compositions can be used.

In a preferred embodiment a magnesium-silver alloy or a mixture of magnesium and silver, or a lithium-aluminum alloy, lithium fluoride-aluminum alloy or a mixture of lithium and aluminum can be used in a film thickness in the range of from 10 nm (100 Å) to 1 μm (10000 Å), preferably from 20 nm (200 Å) to 500 nm (5000 Å).

Such cathodes can be deposited on the foregoing electron transporting layer by known vacuum deposition techniques described above.

In a preferred embodiment of this invention a light-emitting layer can be used between the hole transporting layer and the electron transporting layer. Usually the light-emitting layer is prepared by forming a thin film on the hole transporting layer.

As methods for forming said thin film, there are, for example, the vacuum deposition method, the spin-coating method, the casting method, the Langmuir-Blodgett (“LB”) method and the like. Among these methods, the vacuum deposition method, the spin-coating method and the casting method are particularly preferred in view of ease of operation and cost.

In case of forming a thin film using a composition by means of the vacuum deposition method, the conditions under which the vacuum deposition is carried out are usually strongly dependent on the properties, shape and crystalline state of the compound(s). However, optimum conditions are usually as follows: temperature of the heating boat: 100 to 400° C.; substrate temperature: −100 to 350° C.; pressure: 1.33×10⁴ Pa (1×10² Torr) to 1.33×10⁻⁴ Pa (1×10⁻⁶ Torr) and deposition rate: 1 pm to 6 nm/sec.

In an organic EL element, the thickness of the light emitting layer is one of the factors determining its light emission properties. For example, if a light emitting layer is not sufficiently thick, a short circuit can occur quite easily between two electrodes sandwiching said light emitting layer, and therefor, no EL emission is obtained. On the other hand, if the light emitting layer is excessively thick, a large potential drop occurs inside the light emitting layer because of its high electrical resistance, so that the threshold voltage for EL emission increases. Accordingly, the thickness of the organic light emitting layer is limited to the range of from 5 nm to 5 μm, preferably to the range of from 10 nm to 500 nm.

In the case of forming a light emitting layer by using the spin-coating method and the casting method, ink jet printing method, the coating can be carried out using a solution prepared by dissolving the composition in a concentration of from 0.0001 to 90% by weight in an appropriate organic solvent such as benzene, toluene, xylene, tetrahydrofurane, methyltetrahydrofurane, N,N-dimethylformamide, dichloromethane, dimethylsulfoxide and the like. If the concentration exceeds 90% by weight, the solution usually is so viscous that it no longer permits forming a smooth and homogenous film. On the other hand, if the concentration is less than 0.0001% by weight, the efficiency of forming a film is too low to be economical. Accordingly, a preferred concentration of the composition is within the range of from 0.01 to 80% by weight.

In the case of using the above spin-coating or casting method, it is possible to further improve the homogeneity and mechanical strength of the resulting layer by adding a polymer binder to the solution for forming the light emitting layer. In principle, any polymer binder may be used, provided that it is soluble in the solvent in which the composition is dissolved. Examples of such polymer binders are polycarbonate, polyvinylalcohol, polymethacrylate, polymethylmethacrylate, polyester, polyvinylacetate, epoxy resin and the like. However, if the solid content composed of the polymer binder and the composition exceeds 99% by weight, the fluidity of the solution is usually so low that it is impossible to form a light emitting layer excellent in homogeneity. On the other hand, if the content of the composition is substantially smaller than that of the polymer binder, the electrical resistance of said layer is very large, so that it does not emit light unless a high voltage is applied thereto. Accordingly, the preferred ratio of the polymer binder to the composition is chosen within the range of from 10:1 to 1:50 by weight, and the solid content composed of both components in the solution is preferably within the range of from 0.01 to 80% by weight, and more preferably, within the range of 0.1 to 60% by weight.

As hole-transporting layers known organic hole transporting compounds such as polyvinyl carbazole

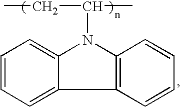

a TPD compound disclosed in J. Amer. Chem. Soc. 90 (1968) 3925:

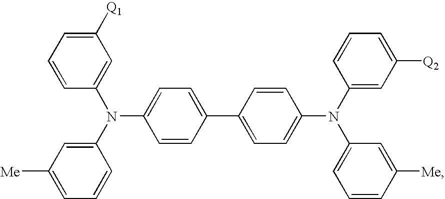

wherein Q₁ and Q₂ each represent a hydrogen atom or a methyl group;
a compound disclosed in J. Appl. Phys. 65(9) (1989) 3610:

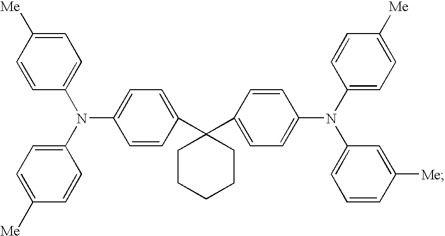

a stilbene based compound

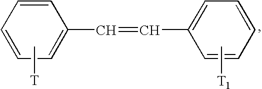

wherein T and T₁ stand for an organic radical;
a hydrazone based compound

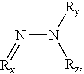

wherein Rx, Ry and Rz stand for an organic radical, and the like can be used.

Compounds to be used as a positive hole transporting material are not restricted to the above listed compounds. Any compound having a property of transporting positive holes can be used as a positive hole transporting material such as triazole derivatives, oxadiazole derivatives, imidazole derivatives, polyarylalkane derivatives, pyrazoline derivative, pyrazolone derivatives, phenylene diamine derivatives, arylamine derivatives, amino substituted chalcone derivatives, oxazole derivatives, stilbenylanthracene derivatives, fluorenone derivatives, hydrazone derivatives, stilbene derivatives, copolymers of aniline derivatives, PEDOT (poly(3,4-ethylenedioxy-thiophene)) and the derivatives thereof, electro-conductive oligomers, particularly thiophene oligomers, porphyrin compounds, aromatic tertiary amine compounds, stilbenyl amine compounds etc.

Particularly, aromatic tertiary amine compounds such as N,N,N′,N′-tetraphenyl-4,4′-diaminobiphenyl, N,N′-diphenyl-N,N′-bis(3-methylphenyl)-4,4′-diaminobiphenyl (TPD), 2,2′-bis(di-p-torylaminophenyl)propane, 1,1′-bis(4-di-torylaminophenyl)-4-phenylcyclohexane, bis(4-dimethylamino-2-methylphenyl)phenylmethane, bis(4-di-p-tolylaminophenyl)phenylmethane, N,N′-diphenyl-N,N′-di(4-methoxyphenyl)-4,4′-diaminobiphenyl, N,N,N′,N′-tetraphenyl-4,4′-diaminodiphenylether, 4,4′-bis(diphenylamino)quaterphenyl, N,N,N-tri(p-tolyl)amine, 4-(di-p-tolylamino)-4′-[4-(di-p-tolylamino)stilyl]stilbene, 4-N,N-diphenylamino-(2-diphenylvinyl)benzene, 3-methoxy-4′-N,N-diphenylaminostilbene, N-phenylcarbazole etc. are used.

Furthermore, 4,4′-bis[N-(1naphtyl)-N-phenylamino]biphenyl disclosed in U.S. Pat. No. 5,061,569 and the compounds disclosed in EP-A 508,562, in which three triphenylamine units are bound to a nitrogen atom, such as 4,4′,4″-tris[N-(3-methylphenyl)-N-phenylamino]triphenylamine, can be used.

A positive hole transporting layer can be formed by preparing an organic film containing at least one positive hole transporting material on the anode. The positive hole transporting layer can be formed by the vacuum deposition method, the spin-coating method, the casting method, the ink jet printing method, the LB method and the like. Of these methods, the vacuum deposition method, the spin-coating method and the casting method are particularly preferred in view of ease and cost.

In the case of using the vacuum deposition method, the conditions for deposition may be chosen in the same manner as described for the formation of a light emitting layer (see above). If it is desired to form a positive hole transporting layer comprising more than one positive hole transporting material, the coevaporation method can be employed using the desired compounds.

In the case of forming a positive hole transporting layer by the spin-coating method or the casting method, the layer can be formed under the conditions described for the formation of the light emitting layer (see above).

As in the case of forming the light emitting layer a smoother and more homogeneous positive hole transporting layer can be formed by using a solution containing a binder and at least one positive hole transporting material. The coating using such a solution can be performed in the same manner as described for the light emitting layer. Any polymer binder may be used, provided that it is soluble in the solvent in which the at least one positive hole transporting material is dissolved. Examples of appropriate polymer binders and of appropriate and preferred concentrations are given above when describing the formation of a light emitting layer.

The thickness of the positive hole transporting layer is preferably chosen in the range of from 0.5 to 1000 nm, preferably from 1 to 100 nm, more preferably from 2 to 50 nm.

As hole injection materials known organic hole transporting compounds such as metal-free phthalocyanine (H₂Pc), copper-phthalocyanine (Cu-Pc) and their derivatives as described, for example, in JP64-7635 can be used. Furthermore, some of the aromatic amines defined as hole transporting materials above, which have a lower ionisation potential than the hole transporting layer, can be used.

A hole injection layer can be formed by preparing an organic film containing at least one hole injection material between the anode layer and the hole transporting layer. The hole injection layer can be formed by the vacuum deposition method, the spin-coating method, the casting method, the LB method and the like. The thickness of the layer is preferably from 5 nm to 5 μm, and more preferably from 10 nm to 100 nm.

The electron transporting materials, which is for example a metal complex compound or a nitrogen-containing five-membered derivative, should have a high electron injection efficiency (from the cathode) and a high electron mobility. The following materials can be exemplified for electron transporting materials: lithium 8-hydroxyquinolinate, zinc bis(8-hydroxyquinolinate), copper bis(8-hydroxyquinolinate), manganese bis(8-hydroxyquinolinate), gallium tris(8-hydroxyquinolinate), tris(8-hydroxyquinolinato)aluminum(III) and its derivatives, such as, for example, aluminum tris(2-methyl-8-hydroxyquinolinate), bis(10-hydroxybenzo[h]quinolinolato)beryllium(II) and its derivatives, zinc bis(10-hydroxybenzo[h]quinolinate), chlorogallium bis(2-methyl-8-quinolinate), gallium bis(2-methyl-8-quinolinate)(o-cresolate), aluminum bis(2-methyl-8-quinolinate)(1-naphtholate), gallium bis(2-methyl-8-quinolinate)(2-naphtholate), gallium bis(2-methyl-8-quinolinate)phenolate, zinc bis(o-(2-benzooxazolyl)phenolate), zinc bis(o-(2-benzothiazolyl)phenolate) and zinc bis(o-(2-benzotrizolyl)phenolate); oxadiazole derivatives, such as 2-(4-biphenyl)-5-(4-tert.-butylphenyl)-1,3,4-oxadiazole and 2,5-bis(1-naphthyl)-1,3,4-oxadiazole, and its dimer systems, such as 1,4-bis[2-(5-phenyloxadiazolyl)]benzene, 1,4-bis[2-(5-phenyloxadiazolyl)-4-tert-butylbenzene], 2,5-bis(1-phenyl)-1,3,4-oxadiazole, 2-(4′-tert-butylphenyl)-5-(4″-biphenyl)1,3,4-oxadiazole, 1,3-bis(4-tert.-butylphenyl-1,3,4)oxadiazolyl)biphenylene and 1,3-bis(4-tert.-butylphenyl-1,3,4-oxadiazolyl)phenylene, oxazole derivatives, dioxazole derivatives, thiazole derivatives, thiadiazole derivatives, triazole derivatives, such as 2,5-bis(1-phenyl)-1,3,4-oxazole, 1,4-bis(2-(4-methyl-5-phenyloxazolyl)benzene, 2,5-bis(1-phenyl)-1,3,4-thiazole, 2-(4′-tert-butylphenyl)-5-(4″-biphenyl)-1,3,4-thiadiazole, 2,5-bis(1-naphthyl)-1,3,4-thiadiazole, 1,4-bis[2-(5-phenylthiazolyl)]benzene, 2-(4′-tert-butylphenyl)-5-(4″-biphenyl)-1,3,4-triazole, or 2,5-bis(1-naphthyl)-1,3,4-triazole and 1,4-bis[2-(5-phenyltriazolyl)]benzene, coumarine derivatives, imidazopyridine derivatives, phenanthroline derivatives or perylene tetracarboxylic acid derivatives disclosed in Appl. Phys. Lett. 48 (2) (1986) 183.

An electron transporting layer can be formed by preparing an organic film containing at least one electron transporting material on the hole transporting layer or on the light-emitting layer. The electron transporting layer can be formed by the vacuum deposition method, the spin-coating method, the casting method, the LB method and the like.

It is preferred that the positive hole inhibiting materials for a positive hole inhibiting layer have high electron injection/transporting efficiency from the electron transporting layer to the light emission layer and also have higher ionisation potential than the light emitting layer to prevent the flowing out of positive holes from the light emitting layer to avoid a drop in luminescence efficiency.

As the positive hole inhibiting material known materials, such as Balq, TAZ and phenanthroline derivatives, e.g. bathocuproine (BCP), can be used:

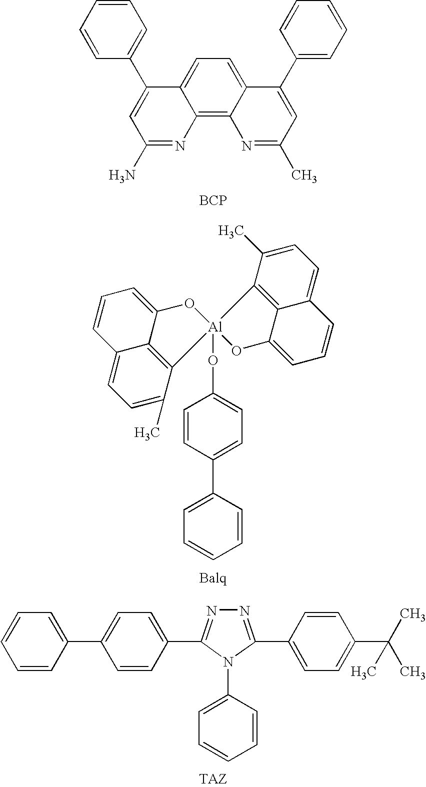

The positive hole inhibiting layer can be formed by preparing an organic film containing at least one positive hole inhibiting material between the electron transporting layer and the light-emitting layer. The positive hole inhibiting layer can be formed by the vacuum deposition method, the spin-coating method, the casting method, ink jet printing method, the LB method and the like. The thickness of the layer preferably is chosen within the range of from 5 nm to 2 μm, and more preferably, within the range of from 10 nm to 100 nm.

As in the case of forming a light emitting layer or a positive hole transporting layer, a smoother and more homogeneous electron transporting layer can be formed by using a solution containing a binder and at least one electron transporting material.

The thickness of an electron transporting layer is preferably chosen in the range of from 0.5 to 1000 nm, preferably from 1 to 100 nm, more preferably from 2 to 50 nm.

The hole-injecting material may be sensitivity-increased by incorporating an electron-accepting material, and the electron-injecting material may be sensitivity-increased by incorporating an electron-donating material.

In the organic EL device of the present invention, the light-emitting layer may contain, in addition to the light-emitting 2H-benzotriazole material of the present invention, at least one of other light-emitting material, other dopant, other hole-injecting material and other electron-injecting material. For improving the organic EL device of the present invention in the stability against temperature, humidity and ambient atmosphere, a protective layer may be formed on the surface of the device, or the device as a whole may be sealed with a silicone oil, or the like.

The electrically conductive material used for the cathode is suitably selected from those having a work function of smaller than 4 eV. The electrically conductive material includes magnesium, calcium, tin, lead, titanium, yttrium, lithium, ruthenium, manganese, aluminum and alloys of these, while the electrically condutive material shall not be limited to these. Examples of the alloys include magnesium/silver, magnesium/indium and lithium/aluminum, while the alloys shall not be limited to these. Each of the anode and the cathode may have a layer structure formed of two layers or more as required.

For the effective light emission of the organic EL device, at least one of the electrodes is desirably sufficiently transparent in the light emission wavelength region of the device. Further, the substrate is desirably transparent as well. The transparent electrode is produced from the above electrically conductive material by a deposition method or a sputtering method such that a predetermined light transmittance is secured. The electrode on the light emission surface side has for instance a light transmittance of at least 10%. The substrate is not specially limited so long as it has adequate mechanical and thermal strength and has transparency. For example, it is selected from glass substrates and substrates of transparent resins such as a polyethylene substrate, a polyethylene terephthalate substrate, a polyether sulfone substrate and a polypropylene substrate.

In the organic EL device of the present invention, each layer can be formed by any one of dry film forming methods such as a vacuum deposition method, a sputtering method, a plasma method and an ion plating method and wet film forming methods such as a spin coating method, a dipping method and a flow coating method. The thickness of each layer is not specially limited, while each layer is required to have a proper thickness. When the layer thickness is too large, inefficiently, a high voltage is required to achieve predetermined emission of light. When the layer thickness is too small, the layer is liable to have a pinhole, etc., so that sufficient light emission brightness is hard to obtain when an electric field is applied. The thickness of each layer is for example in the range of from about 5 nm to about 10 μm, for instance about 10 nm to about 0.2 μm.

In the wet film forming method, a material for forming an intended layer is dissolved or dispersed in a proper solvent such as ethanol, chloroform, tetrahydrofuran and dioxane, and a thin film is formed from the solution or dispersion. The solvent shall not be limited to the above solvents. For improving the film formability and preventing the occurrence of pinholes in any layer, the above solution or dispersion for forming the layer may contain a proper resin and a proper additive. The resin that can be used includes insulating resins such as polystyrene, polycarbonate, polyarylate, polyester, polyamide, polyurethane, polysulfone, polymethyl methacrylate, polymethyl acrylate and cellulose, copolymers of these, photoconductive resins such as poly-N-vinylcarbozole and polysilane, and electroconducting polymers such as polythiophene and polypyrrole. The above additive includes an antioxidant, an ultraviolet absorbent and a plasticizer.

When the light-emitting benzotriazole material of the present invention is used in a light-emitting layer of an organic EL device, an organic EL device can be improved in organic EL device characteristics such as light emission efficiency and maximum light emission brightness. Further, the organic EL device of the present invention is remarkably stable against heat and electric current and gives a usable light emission brightness at a low actuation voltage. The problematic deterioration of conventional devices can be remarkably decreased.

The following Examples illustrate the invention. In the Examples and throughout this application, the term light emitting material means the present 2H-benzotriazole compounds.

### EXAMPLE 1

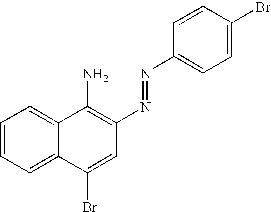

a) 4-Bromoaniline (58.14 mmol) is dissolved in 200 ml water using 174 mmol HCl. The mixture is cooled to 0° C. and sodium nitrite (58.1 mmol) in 30 ml water is added dropwise over 30 minutes. After 45 minutes the diazonium salt is added via cannula to a mixture of 1-amino-4-bromonaphthalene (58.14 mmol) in 300 ml ethanol at 0° C. After 2 hours sodium carbonate (80.2 mmol) in 100 ml water is added dropwise, producing a pH of 7. After an additional 30 minutes the red precipitate is filtered and washed with water (2×300 ml). The brown-red solid was triturated in 100 ml methanol overnight, filtered and dried. The product was dried in vacuo to give a bright red solid (yield: 91%). ¹H NMR (ppm, (CD₃)₂SO): 8.59 (d, 1H), 8.12 (s, 1H), 8.07 (d, 1H), 8.04 (d, 2H), 7.82 (d, 1H), 7.76 (d, 2H), 7.70 (t, 1H).

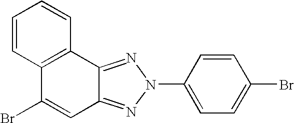

b) The product from step a) (49.37 mmol) and copper(II) acetate (0.49 mmol) are placed in a 250 ml flask with a stir bar. 250 ml tert-amyl alcohol are added and the mixture is heated to 80° C. tert-Butyl hydroperoxide, (98.7 mmol) is slowly added and the reaction is monitored by TLC. The flask is cooled to room temperature and the product is filtered. Washing with tert-amyl alcohol and removal of volatiles in vacuo give a light brown solid. The product is triturated in 30 ml methanol overnight, filtered and dried to give an off-white solid (yield: 77%). ¹H NMR (ppm,, CDCl₃): 8.47 (m, 1H), 8.20 (d, 1H), 8.08 (d, 2H), 8.01 (s, 1H), 7.58 (m, 2H), 7.49 (d, 2H).

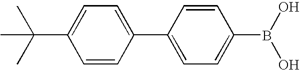

c) Magnesium turnings (68.7 mmol) are dry stirred under argon for one hour. 32 ml ether are added, followed by 2 drops of dibromoethane. 4-Bromo-4′-tert-butylbiphenyl (Murphy, S., et. al. J. Org. Chem. 1995, 60, 2411) (34.6 mmol) in 20 ml ether and 25 ml THF is added dropwise over 1 hour. The mixture is refluxed for 2 hours at 37° C. In a separate flask triisopropylborate (41 mmol) and 30 ml THF are cooled to −78° C. under argon. The above Grignard reagent is added via cannula and the reaction is allowed to stir at −78° C. for 1 hour. The flask is warmed to room temperature and stirred for an additional hour. The mixture is poured into a flask containing HCl/water and is stirred for 2 hours. The beige product is filtered and washed with water. Removal of volatiles in vacuo give an off-white solid (yield: 64%). Tₘ=192° C. ¹H NMR (ppm, (CD₃)₂SO): 7.80 (d, 2H), 7.55 (two overlapping doublets, 4H), 7.41 (d, 2H), 1.25 (s, 9H).

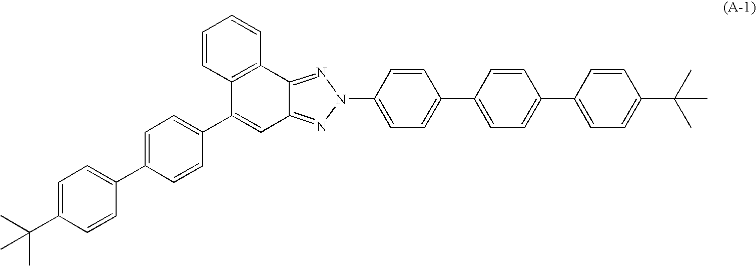

d) The product from step b) (0.94 mmol), the product from step c) (2.83 mmol), palladium tetrakis(triphenylphosphine) (8.6 μmol) and 10 ml N,N-dimethylacetamide are placed in a 100 ml flask and purged with argon for 2 hours. Tetraethylammonium hydroxide (20% in water) is placed in a 50 ml flask and purged with argon for 2 hours. Then 2.0 ml of the base solution (2.8 mmol) are added to the first flask under argon. The mixture is heated to 100° C. overnight and cooled. TLC showed one spot (hexanes:ethyl acetate, 1:1). 20 ml water are added and the product is removed via filtration. Washing with 20 ml water, followed by 20 ml methanol and drying in vacuo give a tan solid (yield: 87%). The product was subsequently purified using zone sublimation. MS (EI): 662 (M+1).

### EXAMPLE 2

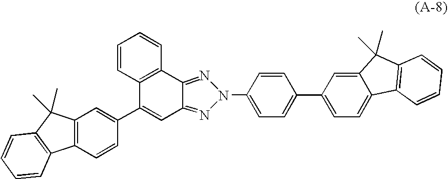

The product from example 1b) (2.48 mmol), 9,9-dimethylfluorene-2-boronic acid (EP-A-1238981, 7.44 mmol), palladium tetrakis(triphenylphosphine) (22 μmol), and 25 ml N,N-dimethylacetamide are placed in a 100 ml flask and purged with argon for 2 hours. Tetraethylammonium hydroxide (20% in water) is placed in a 50 ml flask and purged with argon for 2 hours. Then 5.3 ml of the base solution (7.5 mmol) are added to the first flask under argon. The mixture is heated to 100° C. overnight and cooled. 50 ml water are added and the product is removed via filtration. Washing with 100 ml water, followed by 30 ml methanol and drying in vacuo give a tan solid (yield: 90%). The product is subsequently purified using zone sublimation. MS (EI): 630 (M+1).

### EXAMPLE 3

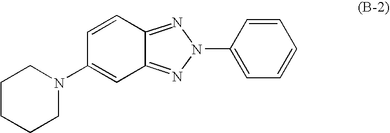

5-Amino-2-phenyl-2H-benzotriazole (Kehrmann, et. al., Chem. Ber. 1892, 25, 899.) (23.8 mmol), and potassium carbonate (52.3 mmole) are placed in a 250 ml flask with 50 ml DMF and a stir bar. While stirring, 1,5-dibromopentane (26.2 mmol) is added via syringe. The mixture is heated to 100° C. for 25 hours. The flask is cooled and the product is extracted using water:dichloromethane. Washing with water and extraction followed by removal of volatiles in vacuo give a dark yellow-green solid. The material is chromatographed using 19:1 hexanes:ethyl acetate (yield: 55%). Tₘ=143° C. ¹H NMR (ppm, CDCl₃): 8.30 (d, 2H), 7.78 (d, 1H), 7.54 (m, 2H), 7.45 (t, 1H), 7.30 (d, 2H), 7.13 (s, 1H), 3.26 (m, 4H), 1.80 (m, 4H), 1.67 (m, 2H). The material has a λₘₐₓ emission of 458 nm in toluene, which corresponds to a color point of CIE (0.139, 0.116).

### EXAMPLE 4

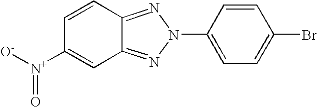

a) Bromophenylhydrazine hydrochloride (0.231 mol), 1-chloro-2,4-dinitrobenzene (0.115 mol) and sodium acetate trihydrate (0.346 mol) are placed in a 1 l reactor. 200 ml Ethanol are added and the mixture is heated to reflux for 6 hours. The mixture is cooled to room temperature and the product is removed by filtration. The material is washed with methanol, water and then methanol again. Volatiles are removed in vacuo to give a light brown solid (yield: 54%). Tₘ=201° C. ¹H NMR (ppm, CDCl₃): 8.94 (d, 1H), 8.30 (overlapping d and dd, 3H), 8.07 (d, 1H), 7.74 (d, 2H).

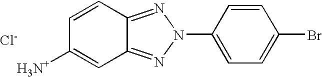

b) The product from example 4a (46.69 mmol) and 200 ml ethanol are placed in a 350 ml reactor. Raney nickel, 8 mL of a 70% slurry, is added. Hydrazine hydrate (0.226 mol) is added in portions over 12 hours with vigorous stirring. After an additional 5 hours at room temperature, the product is filtered and washed with methanol. The material is slurried in 200 ml water and 120 ml concentrated hydrochloric acid (1.44 mol) are added slowly. After stirring for 20 hours, the product is filtered and washed with water. Washing with methanol and removal of volatiles in vacuo give a tan solid (yield: 67%). ¹H NMR (ppm, OS(CD₃)₂): 8.19 (d, 2H), 7.97 (d, 1H), 7.83 (d, 2H), 7.45 (d, 1H), 7.28 (dd, 1H).

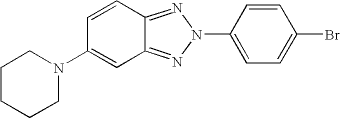

c) The product from example 4b (15.4 mmol) and potassium carbonate (46.5 mmol) are placed in a 250 ml flask with 60 ml DMF. While stirring, 1,5-dibromopentane (19.8 mmol) is added via syringe. The mixture is heated to 100° C. for 2 hours. Additional 1,5-dibromopentane (7.4 mmol) is added via syringe. Heating is continued for 20 hours. The product is extracted using dichloromethane-water and washed with water. The product is dried over silica and chromatographed using 19:1 hexanes:ethyl acetate. The material is isolated as a yellow solid (yield: 46%). ¹H NMR (ppm, CDCl₃): 8.18 (d, 2H), 7.75 (d, 1H), 7.65 (d, 2H), 7.30 (d, 1H), 7.08 (s, 1H), 3.25 (m, 4H), 1.79 (m, 4H), 1.65 (m, 2H).

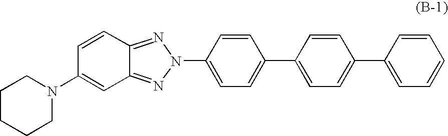

d) The product from step 4c) (4.20 mmol), 4-biphenylboronic acid (2.83 mmol), palladium tetrakis(triphenylphosphine) (35 mmol) and 25 ml N,N-dimethylacetamide are placed in a 100 ml flask and purged with argon for 2 hours. Tetraethylammonium hydroxide, 20% in water, is placed in a 50 mL flask and purged with argon for 2 hours. Then, 4.6 mL of the base solution (6.5 mmol) are added to the first flask under argon. The mixture is heated to 100° C. overnight and cooled. TLC showed two spots (hexanes:ethyl acetate, 1:1). 20 ml Water are added and the product is removed via filtration. Washing with water (20 ml), followed by methanol (20 ml), and drying in vacuo give a yellow solid (yield: 85%). Tₘ=213° C. The product is subsequently purified using zone sublimation. MS (EI): 431 (M+1).

### EXAMPLE 5

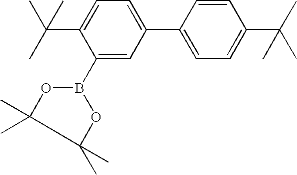

a) 2-Bromo-4,4′-di-tert-butyl-biphenyl (34.8 mmol) is dissolved in THF and the solution is cooled to −75° C. n-Butyl lithium (1.6 mol/l solution, 41.7 mmol) is added to the solution over 10 minutes. The mixture is stirred for 1 hour at −75° C. To the reaction mixture 2-isopropoxy-4,4,5,5-tetramethyl-1,3,2-dioxaborane (69.5 mmol) in THF is added dropwise over 15 minutes at −75° C. The flask is warmed to room temperature and stirred for 2 hours. The mixture is poured into H₂O and extracted with ethylacetate. The organic layer is dried over MgSO₄ and concentrated by evaporation. Column chromatography of crude product with hexane and hexane/ethylacetate (10/1) as eluent gives a white solid (yield: 74.1%). ¹H NMR (ppm, CDCl₃): 7.50 (d, 1H), 7.47 (dd, 1H), 7.34 (d, 2H), 7.27 (d, 1H), 7.17 (d, 2H), 3.28 (s, 12H), 1.23, (s, 9H), 1.06 (s, 9H).

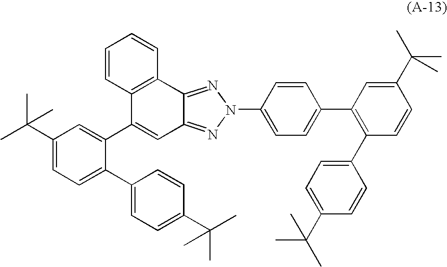

b) The product from example 5b (5.1 mmol), 2-(4,4′-di-tert-butyl biphenyl-2-yl)-4,4,5,5-tetramethyl-1,3,2-dioxaborane (12.7 mmol), palladium tetrakis(triphenylphosphine) (0.25 mmol), tetraethylammonium hydroxide (20% in water, 15.3 mmol) and 60 ml of N,N-dimethylacetamide are placed in a 200 ml flask. The mixture is stirred at 110° C. overnight and cooled. The mixture is then poured into H₂O and extracted with ethylacetate. The organic layer is dried over MgSO₄ and concentrated by evaporation. Column chromatography of the crude product with hexane as eluent gives a greenish black solid (yield: 63.5%). The product is subsequently purified using zone sublimation. ¹H NMR (ppm, CDCl₃): 8.60 (d, 1H), 8.20 (d, 2H), 7.31-7.62 (m, 12H), 7.25 (d, 2H), 7.09 (d, 2H), 7.03 (d, 2H), 6.99 (d, 2H), 1.40 (d, 18H), 1.26 (s, 9H), 1.11 (s, 9H).

### EXAMPLE 6

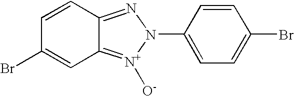

a) 4-Bromophenylhydrazine HCl (0.39 mol), and NaOAc.3 H₂O (0.59 mol) are placed in a 1.5 l flask with EtOH 550 ml. While stirring, 1-fluoro-4-bromo-2-nitrobenzene (0.20 mol) is added (by pouring). The slurry becomes slightly orange. The mixture is heated to reflux overnight (20 hours). After cooling to room temperature, the mixture is filtered and washed with EtOH. Without vacuum on the frit, water is added with stirring to dissolve the NaCl and NaF. Vacuum is applied and the process is repeated. Stirring twice with MeOH in the same manner and applying vacuum give a light yellow, crystalline solid. Volatiles are removed in vacuum. (Yield: 93.4%)

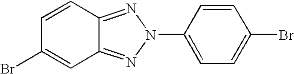

b) The product from example 6a (0.11 mol), and sodium hydrogen sulfite (0.11 mol) are placed in a 250 ml 3 neck round balloon with 200 ml DMF. While stirring, the reaction mixture is heated to 110° C. overnight. After cooling to room temperature, the mixture is poured into 300 ml ice water, and then the precipitate is filtered off and washed with 1000 ml water and 500 ml EtOH. Volatiles are removed in vacuum give a slightly beige powder. (Yield: 93.4%) ¹H NMR (ppm, CDCl₃): 8.21 (dd, 2H), 8.09 (d, 1H), 7.79 (dd, 1H), 7.67 (dd, 2H), 7.49 (dd, 1H).

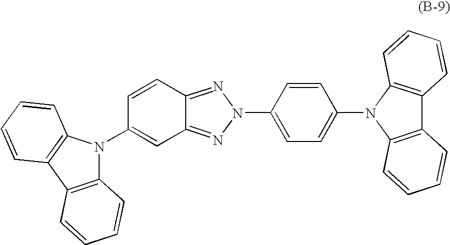

c) The product from example 6b (4.24 mmol), carbazole (8.92 mmol), Cul (9.34 mmol), potassium carbonate (9.34 mmol) and 30 ml of N,N-dimethylacetamide are placed in a 100 ml flask. The mixture is stirred at 170° C. overnight and cooled. Cul is removed by filtration, the mixture is then poured into H₂O and the generated solid is obtained by filtration. The solid is washed with H₂O, EtOH and dried under reduced pressure. Column chromatography of the crude product with hexane as eluent gives a yellow solid (yield: 24.7%). ¹H NMR (ppm, CDCl₃): 8.66 (dd, 2H), 8.19 (m, 6H), 7.83 (dd, 2H), 7.67 (dd, 1H), 7.52 (dd, 4H), 7.46 (td, 4H), 7.34 (td, 4H).

### APPLICATION EXAMPLE 1 (DEVICE)

The following device structure is prepared: ITO/CuPC/NPD/Compound A-1/TPBI/LiF/Al where ITO is indium tin oxide, CuPC is copper phthalocyanine, NPD is 4,4′-bis-(1naphthylphenylamino) biphenyl, and TPBI is 1,3,5-tris-(N-phenyl-benzimidazol-2-yl) benzene. Using this device structure, a maximum brightness of 2200 cd/m² is observed at a maximum efficiency of 0.67 cd/A with an emission λₘₐₓ at 450 nm.

### APPLICATION EXAMPLE 2 (DEVICE)

The following device structure is prepared: ITO/CuPC/NPD/Compound A-8/TPBI/LiF/Al. Using this device structure, a maximum brightness of 3400 cd/m² is observed at a maximum efficiency of 0.83 cd/A with an emission λₘₐₓ at 467 nm.

### APPLICATION EXAMPLE 3 (DEVICE)

The following device structure is prepared: ITO/CuPC/NPD/Compound A-1+Compound B-2 (2.3% by weight)/TPBI/LiF/Al. Using this device structure, a maximum brightness of 6800 cd/m² is observed at a maximum efficiency of 1.6 cd/A with an emission at CIE (0.148, 0.122).

### APPLICATION EXAMPLE 4 (DEVICE)

The following device structure is prepared: ITO/CuPC/NPD/Compound A-1+Compound B-1 (1.6% by weight)/TPBI/LiF/Al. Using this device structure, a maximum brightness of 7600 cd/m² is observed at a maximum efficiency of 1.6 cd/A with an emission at CIE (0.161, 0.131).

### APPLICATION EXAMPLE 5 (DEVICE)

The following device structure is prepared: ITO/CuPC/TCTA/Compound A-13/TPBI/LiF/IA where ITO is indium tin oxide, CuPC is copper phthalocyanine, TCTA is 4,4′,4″-tri-(N-carbazoyl)triphenylamine, and TPBI is 1,3,5-tris-(N-phenyl-benzimidazol-2-yl) benzene. Using this device structure, a brightness of 146 cd/m² is observed with a efficiency of 0.37 cd/A at 12 V with an emission λₘₐₓ at 440 nm.

### APPLICATION EXAMPLE 6 (DEVICE)

The following device structure is prepared: ITO/CuPC/TCTA/Compound A-13+Compound B-9 (1.6% by weight)/TPBI/LiF/Al. Using this device structure, a brightness of 114 cd/m² is observed with a efficiency of 0.53 cd/A at 12 V with an emission λₘₐₓ at 440 nm.

### APPLICATION EXAMPLE 7 (DEVICE)

The following device structure is prepared: ITO/CuPC/TCTA/Compound A-13+Compound D-8 (1.7% by weight)/TPBI/LiF/Al. Using this device structure, a brightness of 161 cd/m² is observed with a efficiency of 0.57 cd/A at 12 V with an emission λₘₐₓ at 437 nm.

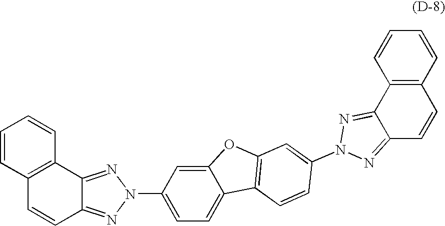

1. A 2H-benzotriazole compound of the formula 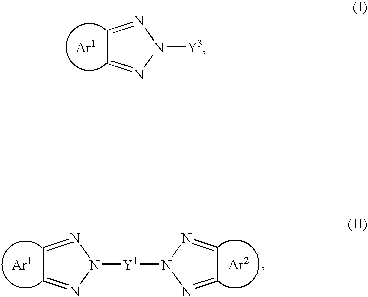
Y¹ is a divalent linking group, and
Y³ is C₁-C₂₅alkyl, C₆-C₃₀aryl, or C₂-C₂₆heteroaryl, which can optionally be substituted, 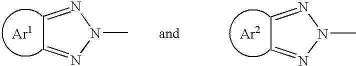
are independently of each other a group of formula 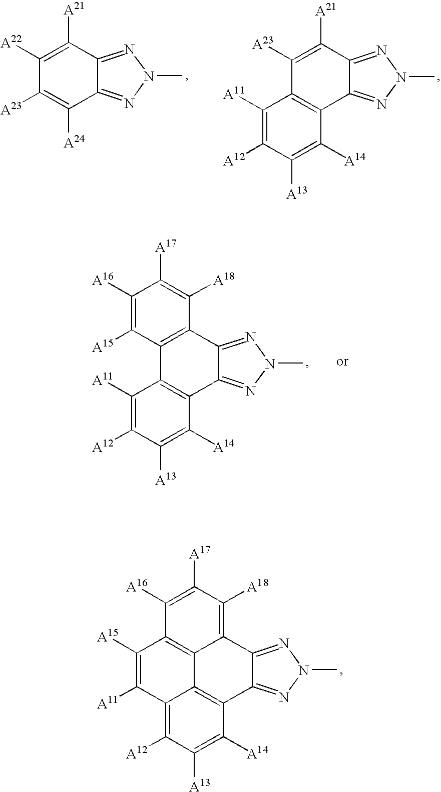
wherein
A²¹, A²², A²³, A²⁴, A¹¹, A¹², A¹³, A¹⁴, A¹⁵, A¹⁶, A¹⁷ and A¹⁸ are independently of each other H, halogen, hydroxy, C₁-C₂₄alkyl, C₁-C₂₄alkyl which is substituted by E and/or interrupted by D, C₁-C₂₄perfluoroalkyl, C₆-C₁₄perfluoroaryl, C₅-C₁₂cycloalkyl, C₅-C₁₂cycloalkyl which is substituted by G and/or interrupted by S—, —O— or —NR²⁵—; —NR²⁵R²⁶, C₁-C₂₄alkylthio, —PR³²R³², C₅-C₁₂cycloalkoxy, C₅-C₁₂cycloalkoxy which is substituted by G, C₆-C₂₄aryl, C₆-C₂₄aryl which is substituted by G, C₁-C₂₄alkyl, C₅-C₁₂cycloalkyl, C₇-C₂₅aralkyl, C₁-C₂₄perfluoroalkyl, C₆-C₁₄perfluoroaryl, or C₁-C₂₄haloalkyl;
C₂-C₂₀heteroaryl, C₂-C₂₀heteroaryl which is substituted by G, fluorine, C₁-C₂₄alkyl, C₅-C₁₂cycloalkyl, C₇-C₂₅aralkyl, C₁-C₂₄perfluoroalkyl, C₆-C₁₄perfluoroaryl, or C₁-C₂₄haloalkyl;
C₂-C₂₄alkenyl, C₂-C₂₄alkynyl, C₁-C₂₄alkoxy, C₁-C₂₄alkoxy which is substituted by E and/or interrupted by D, C₇-C₂₅aralkyl, C₇-C₂₅aralkyl, which is substituted by G, C₇-C₂₅aralkoxy, C₇-C₂₅aralkoxy which is substituted by G, or —CO—R²⁸,
or
A²² and A²³ or A¹¹ and A²³ are a group 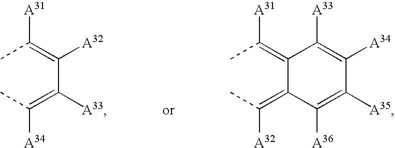
or
two groups A¹¹, A¹², A¹³, A¹⁴ A¹⁵, A¹⁶, A¹⁷ and A¹⁸, which are neighbouring to each other, are a group 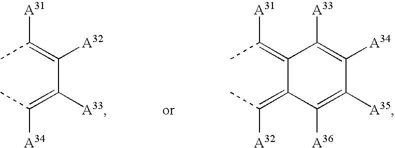
wherein
A³¹, A³², A³³, A³⁴, A³⁵ and A³⁶ are independently of each other H, halogen, hydroxy, C₁-C₂₄alkyl, C₁-C₂₄alkyl which is substituted by E and/or interrupted by D, C₁-C₂₄perfluoroalkyl, C₆-C₁₄perfluoroaryl, C₅-C₁₂cycloalkyl, C₅-C₁₂cycloalkyl which is substituted by G and/or interrupted by S—, —O— or —NR²⁵—; C₅-C₁₂cycloalkoxy, C₅-C₁₂cycloalkoxy which is substituted by G, C₆-C₂₄aryl, C₆-C₂₄aryl which is substituted by G, C₂-C₂₀heteroaryl, C₂-C₂₀heteroaryl which is substituted by G, C₂-C₂₄alkenyl, C₂-C₂₄alkynyl, C₁-C₂₄alkoxy, C₁-C₂₄alkoxy which is substituted by E and/or interrupted by D, C₇-C₂₅aralkyl, C₇-C₂₅aralkyl, which is substituted by G, C₇-C₂₅aralkoxy, C₇-C₂₅aralkoxy which is substituted by G, or —CO—R²⁸;
D is —CO—; —COO—; —S—; —SO—; —SO₂; —O—; —NR²⁵—; —SiR³⁰R³¹—; —POR³²—; —CR²³═CR²⁴—; or —C≡C—; and
E is —OR²⁹; —SR²⁹; —NR²⁵R²⁶; —COR²⁸; —COOR²⁷; —CONR²⁵R²⁶; —CN; —OCOOR²⁷; or halogen;
G is E, or C₁-C₂₄alkyl, wherein
R²³, R²⁴, R²⁵ and R²⁶ are independently of each other H; C₆-C₁₈aryl; C₆-C₁₈aryl which is substituted by C₁-C₂₄alkyl, or C₁-C₂₄alkoxy; C₁-C₂₄alkyl; or C₁-C₂₄alkyl which is interrupted by —O—; or
R²⁵ and R²⁶ together form a five or six membered ring,
R²⁷ and R²⁸ are independently of each other H; C₆-C₁₈aryl; C₆-C₁₈aryl which is substituted by C₁-C₂₄alkyl, or C₁-C₂₄alkoxy; C₁-C₂₄alkyl; or C₁-C₂₄alkyl which is interrupted by —O—,
R²⁹ is H; C₆-C₁₈aryl; C₆-C₁₈aryl, which is substituted by C₁-C₂₄alkyl, or C₁-C₂₄alkoxy; C₁-C₂₄alkyl; or C₁-C₂₄alkyl which is interrupted by —O—,
R³⁰ and R³¹ are independently of each other C₁-C₂₄alkyl, C₆-C₁₈aryl, or C₆-C₁₈aryl, which is substituted by C₁-C₂₄alkyl, and
R³² is C₁-C₂₄alkyl, C₆-C₁₈aryl, or C₆-C₁₈aryl which is substituted by C₁-C₂₄alkyl.

2. A 2H-benzotriazole compound according to claim 1, wherein at least one of the substituents A²¹, A²², A²³, A²⁴, A¹¹, A¹², A¹³, A¹⁴, A¹⁵, A¹⁶, A¹⁷ and A¹⁸ is a group of formula 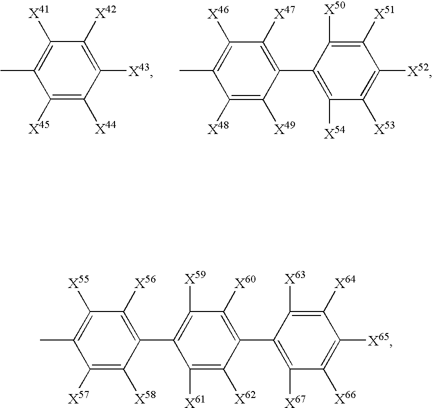
wherein X⁴¹, X⁴², X⁴³, X⁴⁴, X⁴⁵, X⁴⁶, X⁴⁷, X⁴⁸, X⁴⁹, X⁵⁰, X⁵¹, X⁵², X⁵³, X⁵⁴, X⁵⁵, X⁵⁶, X⁵⁷, X⁵⁸, X⁵⁹, X⁶⁰, X⁶¹, X⁶², X⁶³, X⁶⁴, X⁶⁵, X⁶⁶ and X⁶⁷ are independently of each other H, fluorine, —NR²⁵R²⁶, C₁-C₂₄alkyl, C₅-C₁₂cycloalkyl, C₇-C₂₅aralkyl, C₁-C₂₄perfluoroalkyl, C₅-C₁₄perfluoroaryl, or C₁-C₂₄haloalkyl, C₁-C₂₄alkyl, which is optionally substituted by E and/or interrupted by D, C₁-C₂₄alkenyl, which is optionally substituted by E, C₅-C₁₂cycloalkyl, which is optionally substituted by G, C₅-C₁₂cycloalkoxy, which is optionally substituted by G, C₆-C₁₈aryl, which is optionally substituted by G, C₁-C₂₄alkoxy, which is optionally substituted by E and/or interrupted by D, C₆-C₁₈aryloxy, which is optionally substituted by G, C₇-C₁₈arylalkoxy, which is optionally substituted by G, C₁-C₂₄alkylthio, which is optionally substituted by E and/or interrupted by D, C₂-C₂₀heteroaryl which is substituted by G, or C₆-C₁₈aralkyl, which is optionally substituted by G, or
X⁴³, X⁶⁵ or X⁵² are a group of formula 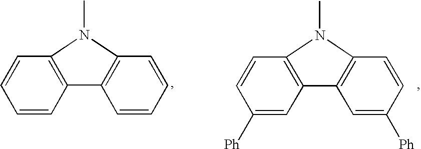
or
two groups X⁴¹, X⁴², X⁴³, X⁴⁴, X⁴⁵, X⁴⁶, X⁴⁷, X⁴⁸, X⁴⁹, X⁵⁰, X⁵¹, X⁵², X⁵³, X⁵⁴, X⁵⁵, X⁵⁶, X⁵⁷, X⁵⁸, X⁵⁹, X⁶⁰, X⁶¹, X⁶², X⁶³, X⁶⁴, X⁶⁵, X⁶⁶ and X⁶⁷, which are neighbouring to each other, are a group 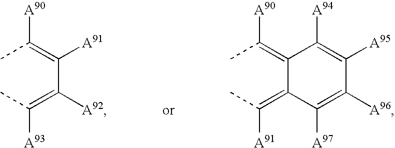
wherein A⁹⁰, A⁹¹, A⁹², A⁹³, A⁹⁴, A⁹⁵, A⁹⁶ and A⁹⁷ are independently of each other H, halogen, hydroxy, C₁-C₂₄alkyl, C₁-C₂₄alkyl which is substituted by E and/or interrupted by D, C₁-C₂₄perfluoroalkyl, C₆-C₁₄perfluoroaryl, C₅-C₁₂cycloalkyl, C₅-C₁₂cycloalkyl which is substituted, by G and/or interrupted by S—, —O—, or —NR²⁵—, C₅-C₁₂cycloalkoxy, C₅-C₁₂cycloalkoxy which is substituted by G, C₆-C₂₄aryl, C₆-C₂₄aryl which is substituted by G, C₂-C₂₀heteroaryl, C₂-C₂₀heteroaryl which is substituted by G, C₂-C₂₄alkenyl, C₂-C₂₄alkynyl, C₁-C₂₄alkoxy, C₁-C₂₄alkoxy which is substituted by E and/or interrupted by D, C₇-C₂₅aralkyl, C₇-C₂₅aralkyl, which is substituted by G, C₇-C₂₅aralkoxy, C₇-C₂₅aralkoxy which is substituted by E, or —CO—R²⁸.

3. A 2H-benzotriazole compound according to claim 1, wherein at least one of the substituents A²¹, A²², A²³, A²⁴, A¹¹, A¹², A¹³, A¹⁴, A¹⁵, A¹⁶, A¹⁷ and A¹⁸ is a group of formula 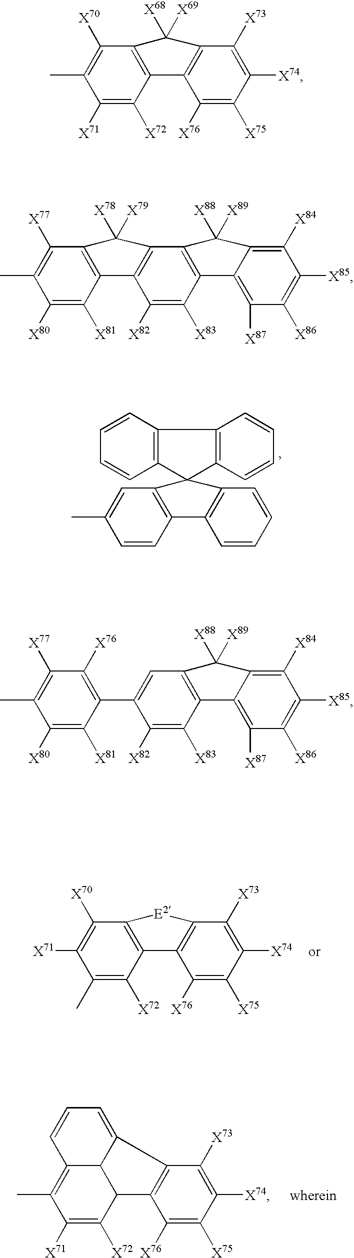
X⁶⁸, X⁶⁹, X⁷⁸, X⁷⁹, X⁸⁸ and X⁸⁹ are independently of each other C₁-C₂₄alkyl which can be interrupted by one or two oxygen atoms,
X⁷⁰, X⁷¹, X⁷², X⁷³, X⁷⁴, X⁷⁵, X⁷⁶, X⁷⁷, X⁸⁰, X⁸¹, X⁸², X⁸³, X⁸⁴, X⁸⁵, X⁸⁶ and X⁸⁷ are independently of each other H, CN, C₁-C₂₄alkyl, C₆-C₁₀aryl, C₁-C₂₄alkoxy, C₁-C₂₄alkylthio, —NR²⁵R²⁶, —CONR²⁵R²⁶, or —COOR²⁷, wherein
R²⁵ and R²⁶ are independently of each other H, C₆-C₁₈aryl, C₇-C₁₈aralkyl, or C₁-C₂₄alkyl, and R²⁷ is C₁-C₂₄alkyl, or
R²⁵ and R²⁶ together form a five or six membered ring, and
E^{2′} is —S—, —O— or —NR^{25′}—, wherein R^{25′} is C₁-C₂₄alkyl, or C₆-C₁₀aryl.

4. A 2H-benzotriazole compound according to claim 1, wherein Y³ is a group of formula 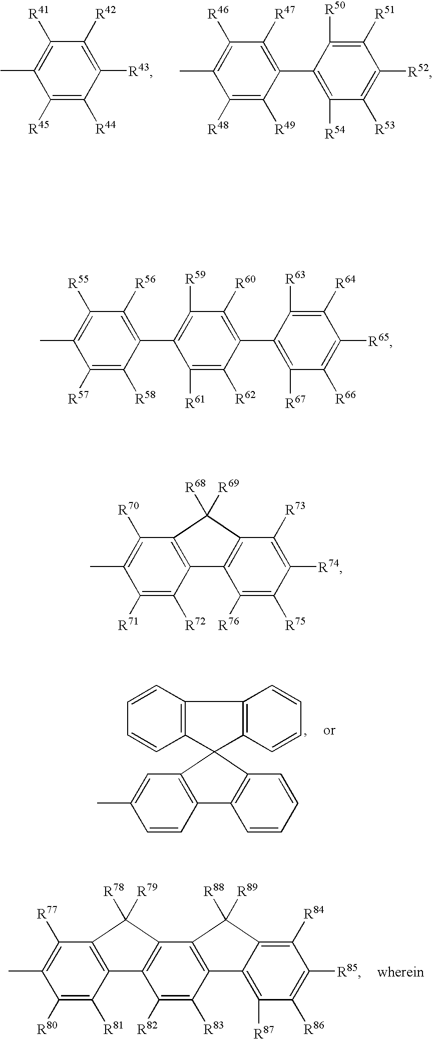
R⁴¹, R⁴², R^{43, R}⁴⁴, R⁴⁵, R⁴⁶, R⁴⁷, R⁴⁸, R⁴⁹, R⁵⁰, R⁵¹, R⁵², R⁵³, R⁵⁴, R⁵⁵, R⁵⁶, R⁵⁷, R⁵⁸, R⁵⁹, R⁶⁰, R⁶¹, R⁶², R⁶³, R⁶⁴, R⁶⁵, R⁶⁶, R⁶⁷, R⁷⁰, R⁷¹, R⁷², R⁷³, R⁷⁴, R⁷⁵, R⁷⁶, R⁷⁷, R⁸⁰, R⁸¹, R⁸², R⁸³, R⁸⁴, R⁸⁵, R⁸⁶, and R⁸⁷ are independently of each other H, fluorine, C₁-C₂₄perfluoroalkyl, C₆-C₁₄perfluoroaryl, —NR²⁵R²⁶, C₁-C₂₄alkyl, which is optionally substituted by E and/or interrupted by D, C₁-C₂₄alkenyl, which is optionally substituted by E, C₅-C₁₂cycloalkyl, which is optionally substituted by G, C₅-C₁₂cycloalkoxy, which is optionally substituted by G, C₆-C₁₈aryl, which is optionally substituted by G, C₁-C₂₄alkoxy, which is optionally substituted by E and/or interrupted by D, C₆-C₁₈aryloxy, which is optionally substituted by G, C₇-C₁₈arylalkoxy, which is optionally substituted by G, C₁-C₂₄alkylthio, which is-optionally substituted by E and/or interrupted by D, C₂-C₂₀heteroaryl which is substituted by G-, or C₆-C₁₈aralkyl, which is optionally substituted by G,
or
R⁴³, R⁶⁵ or R⁵² are a group of formula 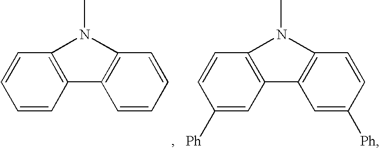
or
two groups R⁴¹, R⁴², R⁴³, R⁴⁴, R⁴⁵, R⁴⁶, R⁴⁷, R⁴⁸, R⁴⁹, R⁵⁰, R⁵¹, R⁵², R⁵³, R⁵⁴, R⁵⁵, R⁵⁶, R⁵⁷, R⁵⁸, R⁵⁹, R⁶⁰, R⁶¹, R⁶², R⁶³, R⁶⁴, R⁶⁵, R⁶⁶, R⁶⁷, R⁷⁰, R⁷¹, R⁷², R⁷³, R⁷⁴, R⁷⁵, R⁷⁶, R⁷⁷, R⁸⁰, R⁸¹, R⁸², R⁸³, R⁸⁴, R⁸⁵, R⁸⁶, and R⁸⁷, which are neighbouring to each other, are a group 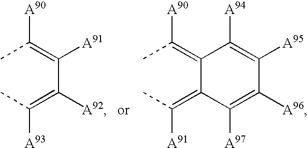
wherein A⁹⁰, A⁹¹, A⁹², A⁹³, A⁹⁴, A⁹⁵, A⁹⁶ and A⁹⁷ are independently of each other H, halogen, —NR²⁵R²⁶, hydroxy, C₁-C₂₄alkyl, C₁-C₂₄alkyl which is substituted by E and/or interrupted by D, C₁-C₂₄perfluoroalkyl, C₆-C₁₄perfluoroaryl, C₅-C₁₂cycloalkyl, C₅-C₁₂cycloalkyl which is substituted by G and/or interrupted by S—, —O— or —NR²⁵—; C₅-C₁₂cycloalkoxy, C₅-C₁₂cycloalkoxy which is substituted by G, C₆-C₂₄aryl, C₆-C₂₄aryl which is substituted by G, C₂-C₂₀heteroaryl, C₂-C₂₀heteroaryl which is substituted by G, C₂-C₂₄alkenyl, C₂-C₂₄alkynyl, C₁-C₂₄alkoxy, C₁-C₂₄alkoxy which is substituted by E and/or interrupted by D, C₇-C₂₅aralkyl, C₇-C₂₅aralkyl, which is substituted by G, C₇-C₂₅aralkoxy, C₇-C₂₅aralkoxy which is substituted by G, or —CO—R²⁸,
R⁶⁸, R⁶⁹, R⁷⁸, R⁷⁹, R⁸⁸ and R⁸⁹ are independently of each other C₁-C₁₈ alkyl, C₁-C₂₄alkyl which is substituted by E and/or interrupted by D, C₆-C₂₄aryl, C₆-C₂₄aryl which is substituted by G, C₂-C₂₀heteroaryl, C₂-C₂₀heteroaryl which is substituted by G, C₂-C₂₄alkenyl, C₂-C₂₄alkynyl, C₁-C₂₄alkoxy, C₁-C₂₄alkoxy which is substituted by E and/or interrupted by D, or C₇-C₂₅aralkyl, or
R⁶⁸ and R⁶⁹, R⁷⁸ and R⁷⁹, and/or R⁸⁸ and R⁸⁹ form a ring, or
R⁶⁸ and R⁷⁰, R⁶⁹ and R⁷³, R⁷⁷ and R⁷⁸ and/or R⁸⁴ and R⁸⁹ are a group 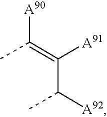
D is —CO—; —COO—; —S—; —SO—; —SO₂—; —O—; —NR²⁵—; —SiR³⁰R³¹—; —POR³²—; —CR²³═CR²⁴—; or —C≡C—; and
E is —OR²⁹; —SR²⁹; —NR²⁵R²⁶; —COR²⁸; —COOR²⁷; —CONR²⁵R²⁶; —CN; —OCOOR²⁷; or halogen;
G is E, or C₁-C₂₄alkyl; wherein
R²³, R²⁴, R²⁵ and R²⁶ are independently of each other H; C₆-C₁₈aryl; C₆-C₁₈aryl which is substituted by C₁-C₂₄alkyl, or C₁-C₂₄alkoxy; C₁-C₂₄alkyl; or C₁-C₂₄alkyl which is interrupted by —O—; or
R²⁵ and R²⁶ together form a five or six membered ring,
R²⁷ and R²⁸ are independently of each other H; C₆-C₁₈aryl; C₆-C₁₈aryl which is substituted by C₁-C₂₄alkyl or C₁-C₂₄alkoxy; C₁-C₂₄alkyl; or C₁-C₂₄alkyl which is interrupted by —O—,
R²⁹ is H; C₆-C₁₈aryl; C₆-C₁₈aryl which is substituted by C₁-C₂₄alkyl or C₁-C₂₄alkoxy; C₁-C₂₄alkyl; or C₁-C₂₄alkyl which is interrupted by —O—,
R³⁰ and R³¹ are independently of each other C₁-C₂₄alkyl, C₆-C₁₈aryl, or C₆-C₁₈aryl, which is substituted by C₁-C₂₄alkyl, and
R³² is C₁-C₂₄alkyl, C₆-C₁₈aryl, or C₆-C₁₈aryl which is substituted by C₁-C₂₄alkyl, or
R⁴³, or R⁵² are a group of formula 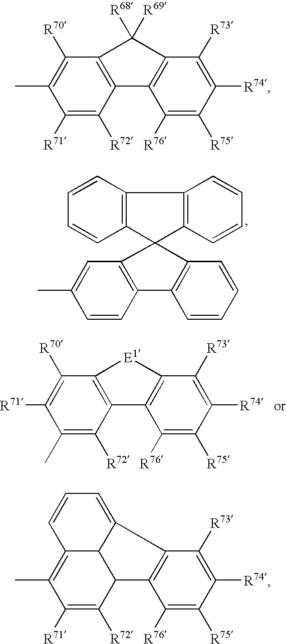
wherein
R^{68′} and R^{69′} are independently of each other C₁-C₂₄alkyl which can be interrupted by one or two oxygen atoms,
R^{70′}, R^{71′}, R^{72′}, R^{73′}, R^{74′}, R^{75′} and R^{76′} are independently of each other H, CN, C₁-C₂₄alkyl, C₆-C₁₀aryl, C₁-C₂₄alkoxy, C₁-C₂₄alkylthio, —NR^{25′}R^{26′}, —CONR^{25′}R^{26′}, or —COOR^{27′},
R^{25′} and R^{26′} are independently of each other H, C₆-C₁₈aryl, C₇-C₁₈aralkyl, or C₁-C₂₄alkyl, and R^{27′} is C₁-C₂₄alkyl; and
E^{1′} is —S—, —O— or —NR^{25′}—, wherein R^{25′} is C₁-C₂₄alkyl, or C₆-C₁₀aryl.

5. A 2H-benzotriazole compound to claim 1, wherein Y¹ is a group of formula 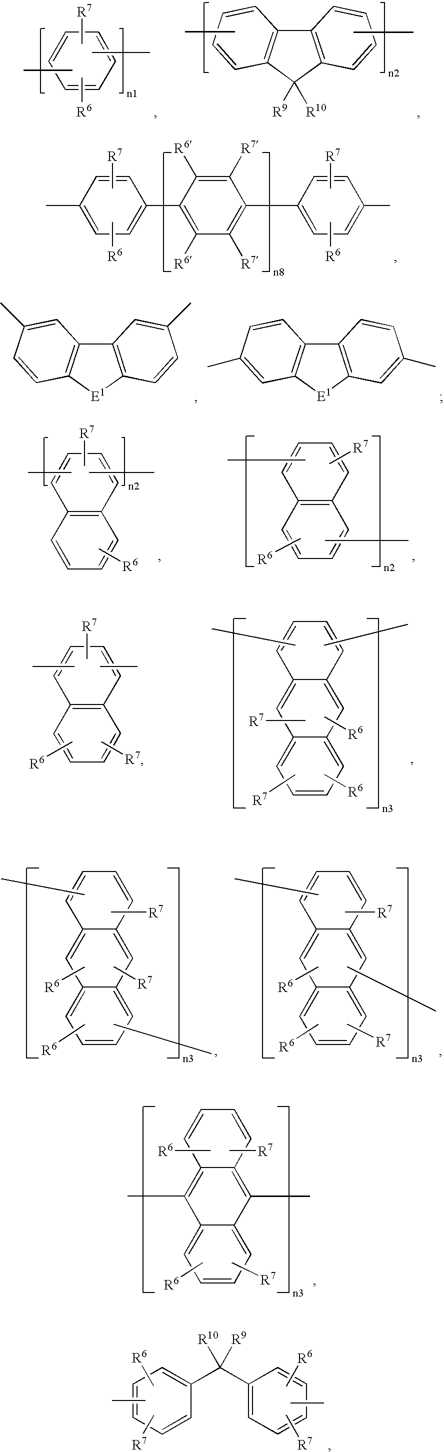 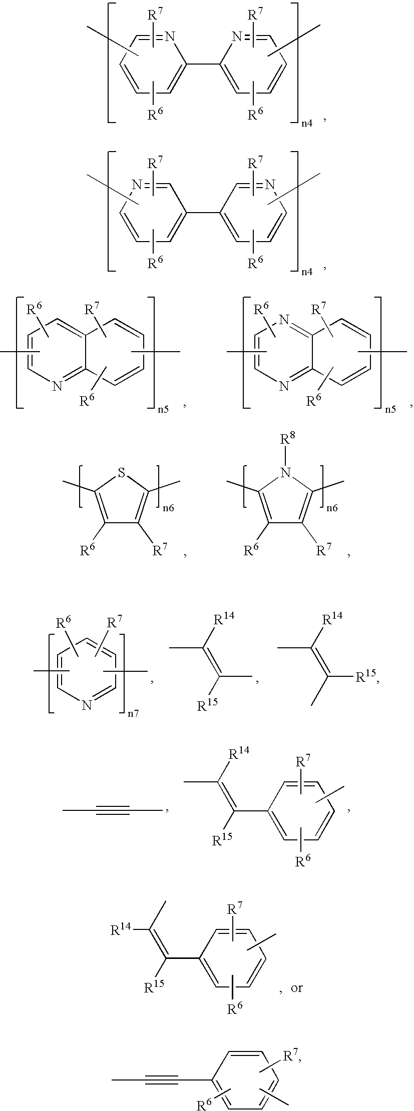
wherein
n1, n2, n3, n4, n5, n6, n7 and n8 are 1, 2, or 3,
E¹ is —S—, —O— or —NR^{25′}—, wherein R^{25′} is C₁-C₂₄alkyl or C₆-C₁₀aryl,
R⁶ and R⁷ are independently of each other H, halogen, —NR²⁵R²⁶, hydroxy, C₁-C₂₄alkyl, C₁-C₂₄alkyl which is substituted by E and/or interrupted by D, C₁-C₂₄perfluoroalkyl, C₆-C₁₄perfluoroaryl, C₅-C₁₂cycloalkyl, C₅-C₁₂cycloalkyl which is substituted by G and/or interrupted by S—, —O— or —NR²⁵—; C₅-C₁₂cycloalkoxy, C₅-C₁₂cycloalkoxy which is substituted by G, C₆-C₂₄aryl, C₆-C₂₄aryl which is substituted by G, C₂-C₂₀heteroaryl, C₂-C₂₀heteroaryl which is substituted by G, C₂-C₂₄alkenyl, C₂-C₂₄alkynyl, C₁-C₂₄alkoxy, C₁-C₂₄alkoxy which is substituted by E and/or interrupted by D, C₇-C₂₅aralkyl, C₇-C₂₅aralkyl, which is substituted by G, C₇-C₂₅aralkoxy, C₇-C₂₅aralkoxy which is substituted by G, or —CO—R²⁸,
R^{6′} and R^{7′} have the meaning of R⁶, or together form a group 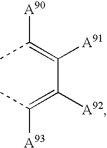
wherein
A⁹⁰, A⁹¹, A⁹², and A⁹³ are independently of each other H, halogen, hydroxy, C₁-C₂₄alkyl, C₁-C₂₄alkyl which is substituted by E and/or interrupted by D, C₁-C₂₄perfluoroalkyl, C₆-C₁₄perfluoroaryl, C₅-C₁₂cycloalkyl, C₅-C₁₂cycloalkyl which is substituted by G and/or interrupted by S—, —O— or —NR²⁵—; C₅-C₁₂cycloalkoxy, C₅-C₁₂cycloalkoxy which is substituted by G, C₆-C₂₄aryl, C₆-C₂₄aryl which is substituted by G, C₂-C₂₀heteroaryl, C₂-C₂₀heteroaryl which is substituted by G, C₂-C₂₄alkenyl, C₂-C₂₄alkynyl, C₁-C₂₄alkoxy, C₁-C₂₄alkoxy which is substituted by E and/or interrupted by D, C₇-C₂₅aralkyl, C₇-C₂₅aralkyl, which is substituted by G, C₇-C₂₅aralkoxy, C₇-C₂₅aralkoxy which is substituted by E, or -CO—R²⁸,
R⁸ is C₁-C₂₄alkyl, C₁-C₂₄alkyl which is substituted by E and/or interrupted by D, C₆-C₂₄ aryl, or C₇-C₂₅aralkyl,
R⁹ and R¹⁰ are independently of each other C₁-C₂₄alkyl, C₁-C₂₄alkyl which is substituted by E and/or interrupted by D, C₆-C₂₄aryl, C₆-C₂₄aryl which is substituted by G, C₂-C₂₀heteroaryl, C₂-C₂₀heteroaryl which is substituted by G, C₂-C₂₄alkenyl, C₂-C₂₄alkynyl, C₁-C₂₄alkoxy, C₁-C₂₄alkoxy which is substituted by E and/or interrupted by D, or C₇-C₂₅aralkyl, or
R⁹ and R¹⁰ form a ring,
R¹⁴ and R¹⁵ are independently of each other H, C₁-C₂₄alkyl, C₁-C₂₄alkyl which is substituted by E and/or interrupted by D, C₆-C₂₄aryl, C₆-C₂₄aryl which is substituted by G, C₂-C₂₀heteroaryl, or C₂-C₂₀heteroaryl which is substituted by G,
D is —CO—, —COO—, —S—, —SO—, —SO₂—, —O—, —NR²⁵—, —SiR³⁰R³¹—, —POR³²—, —CR²³═CR²⁴—, or —C≡C—, G is E or C₁-C₂₄alkyl, and
E is —OR²⁹, SR²⁹, —NR²⁵R²⁶, —COR²⁸, —COOR²⁷, —CONR²⁵R²⁶, —CN, —OCOOR²⁷, or halogen, wherein
R²³, R²⁴, R²⁵ and R²⁶ are independently of each other H, C₆-C₁₈aryl, C₆-C₁₈aryl which is substituted by C₁-C₂₄alkyl, C₁-C₂₄alkoxy, C₁-C₂₄alkyl, or C₁-C₂₄alkyl which is interrupted by —O—,
or
R²⁷ and R²⁸ are independently of each other H, C₆-C₁₈aryl, C₆-C₁₈aryl which is substituted by C₁-C₂₄alkyl, or C₁-C₂₄alkoxy, C₁-C₂₄alkyl, or C₁-C₂₄alkyl which is interrupted by —O—,
R²⁹ is H, C₆-C₁₈aryl, C₆-C₁₈aryl which is substituted by C₁-C₂₄alkyl, C₁-C₂₄alkoxy, C₁-C₂₄alkyl, or C₁-C₂₄alkyl which is interrupted by —O—,
R³⁰ and R³¹ are independently of each other C₁-C₂₄alkyl, C₆-C₁₈aryl, or C₆-C₁₈aryl which is substituted by C₁-C₂₄alkyl, and
R³² is C₁-C₂₄alkyl, C₆-C₁₈aryl, or C₆-C₁₈aryl which is substituted by C₁-C₂₄alkyl.

6. A 2H-benzotriazole compound to claim 1, wherein the 2H-benzotriazole compound is a compound of formula 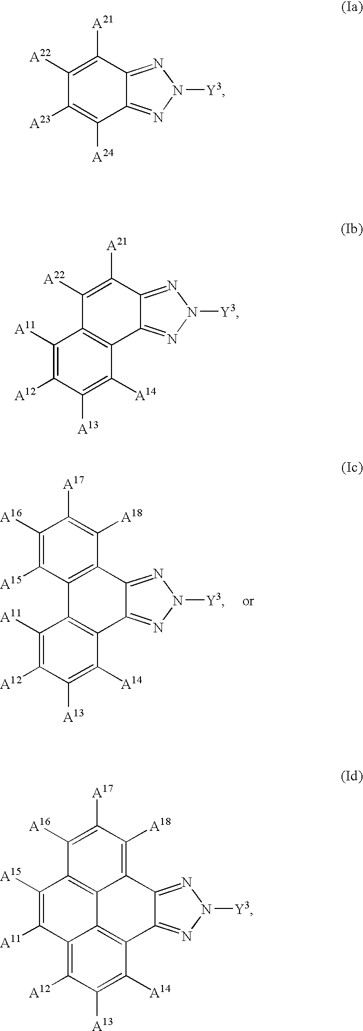
wherein A¹² or A²³ are a group of formula 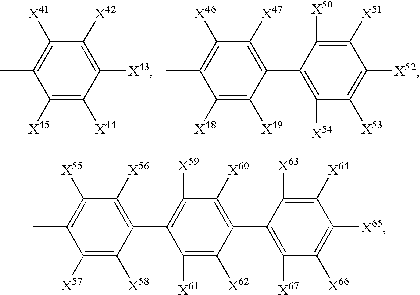
wherein X⁴¹, X⁴², X⁴³, X⁴⁴, X⁴⁵, X⁴⁶, X⁴⁷, X⁴⁸, X⁴⁹, X⁵⁰, X⁵¹, X⁵², X⁵³, X⁵⁴, X⁵⁵, X⁵⁶, X⁵⁷, X⁵⁸, X⁵⁹, X⁶⁰, X⁶¹, X⁶², X⁶³, X⁶⁴, X⁶⁵, X⁶⁶ and X⁶⁷ are independently of each H, CN, fluorine, C₁-C₂₄alkyl, C₅-C₁₂cycloalkyl, C₇-C₂₅aralkyl, C₁-C₂₄perfluoroalkyl, C₆-C₁₄perfluoroaryl, C₁-C₂₄haloalkyl, C₆-C₁₀aryl, which can optionally be substituted by one or more C₁-C₈alkyl or C₁-C₈alkoxy groups; C₁-C₂₄alkoxy, C₁-C₂₄alkylthio, —NR²⁵R²⁶, —CONR²⁵R²⁶, or —COOR²⁷,
or
two groups X⁴¹, X⁴², X⁴³, X⁴⁴, X⁴⁵, X⁴⁶, X⁴⁷, X⁴⁸, X⁴⁹, X⁵⁰, X⁵¹, X⁵², X⁵³, X⁵⁴, X⁵⁵, X⁵⁶, X⁵⁷, X⁵⁸, X⁵⁹, X⁶⁰, X⁶¹, X⁶², X⁶³, X⁶⁴, X⁶⁵, X⁶⁶ and X⁶⁷, which are neighbouring to each other, are a group 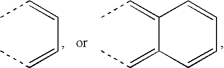
or A¹² and A²³ are a group of formula 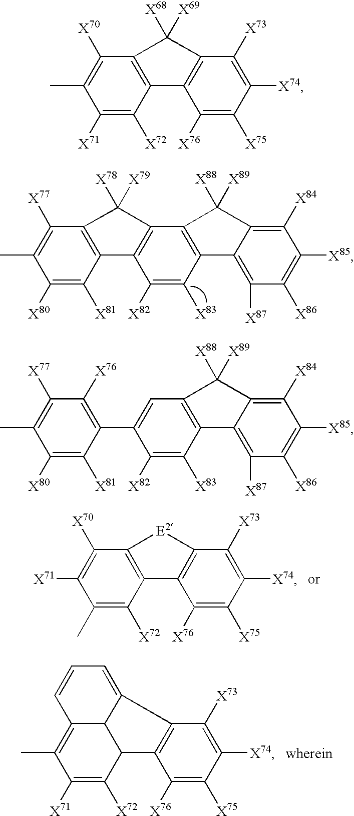
X⁶⁸, X⁶⁹, X⁷⁸, X⁷⁹, X⁸⁸ and X⁸⁹ are independently of each other C₁-C₂₄alkyl which can be interrupted by one or two oxygen atoms,
X⁷⁰, X⁷¹, X⁷², X⁷³, X⁷⁴, X⁷⁵, X⁷⁶, X⁷⁷, X⁸⁰, X⁸¹, X⁸², X⁸³, X⁸⁴, X⁸⁵, X⁸⁶ and X⁸⁷ are independently of each other H, CN, C₁-C₂₄alkyl, C₆-C₁₀aryl which can optionally be substituted by one or more C₁-C₈alkyl or C₁-C₈alkoxy groups; C₁-C₂₄alkoxy, C₁-C₂₄alkylthio, —NR²⁵R²⁶, —CONR²⁵R²⁶, or —COOR²⁷,
E^{2′} is —S—, —O— or —NR^{25′}—, wherein R^{25′} is C₁-C₂₄alkyl, or C₆-C₁₀aryl,
A²¹, A²² and A²⁴ are independently of each other hydrogen, halogen, C₁-C₂₄alkyl, C₁-C₂₄perfluoroalkyl, C₆-C₁₄perfluoroaryl, C₅-C₁₂cycloalkyl, C₇-C₂₅aralkyl, C₁-C₂₄haloalkyl, C₆-C₁₈aryl, which can optionally be substituted by one or more C₁-C₈alkyl or C₁-C₈alkoxy groups; —NR²⁵R²⁶, —CONR²⁵R²⁶, or —COOR²⁷, or C₂-C₁₀heteroaryl or
A²² and A²³ or A¹¹ and A²³ are a group of formula 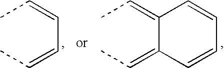
A¹¹, A¹³, A¹⁴, A¹⁵, A¹⁶, A¹⁷, and A¹⁸ are independently of each other H, CN, C₁-C₂₄alkyl, C₅-C₁₂cycloalkyl, C₇-C₂₅aralkyl, C₁-C₂₄perfluoroalkyl, C₆-C₁₄perfluoroaryl, C₁-C₂₄haloalkyl, C₁-C₂₄alkoxy, C₁-C₂₄alkylthio, C₆-C₁₈aryl, —NR²⁵R²⁶, —CONR²⁵R²⁶, or —COOR²⁷, or C₂-C₁₀heteroaryl, wherein
R²⁵ and R²⁶ are independently of each other H, C₆-C₁₈aryl, C₇-C₁₈aralkyl, or C₁-C₂₄alkyl, R²⁷ is C₁-C₂₄alkyl, and
Y³ is a group of formula 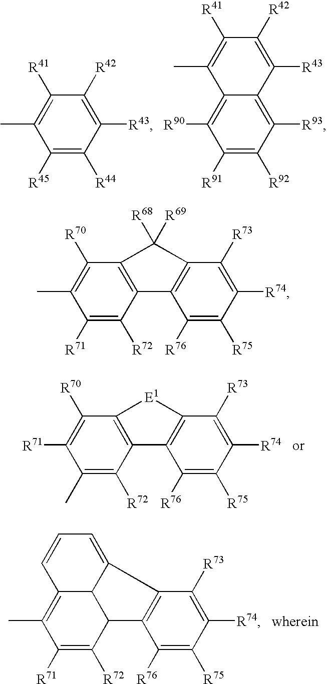
R⁴¹ is hydrogen, C₁-C₂₄alkoxy or —OC₇-C₁₈aralkyl,
R⁴² is hydrogen or C₁-C₂₄alkyl,
R⁴³ is hydrogen, halogen, —CONR²⁵R²⁶, —COOR²⁷, 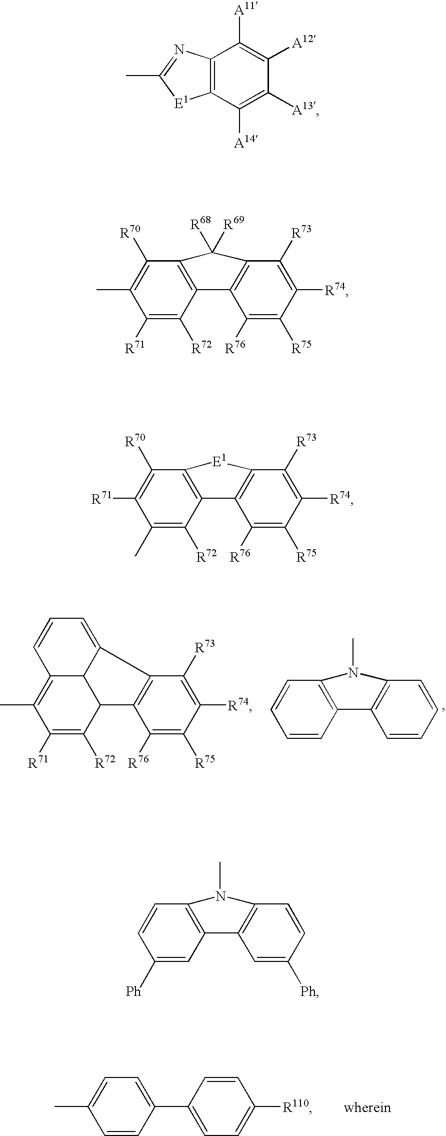
A^{11′}, A^{12′}, A^{13′}, and A^{14′} are independently of each other H, CN, C₁-C₂₄alkyl, C₁-C₂₄alkoxy, C₁-C₂₄alkylthio, —NR²⁵R²⁶, —CONR²⁵R²⁶, or —COOR²⁷,
E¹ is —S—, —O— or —NR^{25′}—, wherein R^{25′} is C₁-C₂₄alkyl or C₆-C₁₀aryl,
R¹¹⁰ is H, CN, C₁-C₂₄alkyl, C₁-C₂₄alkoxy, C₁-C₂₄alkylthio, —NR²⁵R²⁶, —CONR²⁵R²⁶, or —COOR²⁷, or
R⁴² and R⁴³ are a group of formula 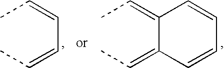
R⁴⁴ is hydrogen, or C₁-C₂₄alkyl,
R⁴⁵ is hydrogen, or C₁-C₂₄alkyl,
R⁶⁸ and R⁶⁹ are independently of each other C₁-C₂₄alkyl, which can be interrupted by one or two oxygen atoms,
R⁷⁰, R⁷¹, R⁷², R⁷³, R⁷⁴, R⁷⁵, R⁷⁶, R⁹⁰, R⁹¹, R⁹², and R⁹³ are independently of each other H, CN, C₁-C₂₄alkyl, C₆-C₁₀aryl, C₁-C₂₄alkoxy, C₁-C₂₄alkylthio, —NR²⁵R²⁶, —CONR²⁵R²⁶, or —COOR²⁷,
R²⁵ and R²⁶ are independently of each other H, C₆-C₁₈aryl, C₇-C₁₈aralkyl, or C₁-C₂₄alkyl, and R²⁷ is C₁-C₂₄alkyl.

7. A 2H-benzotriazole compound according to claim 1, wherein the 2H-benzotriazole compound is a compound of formula 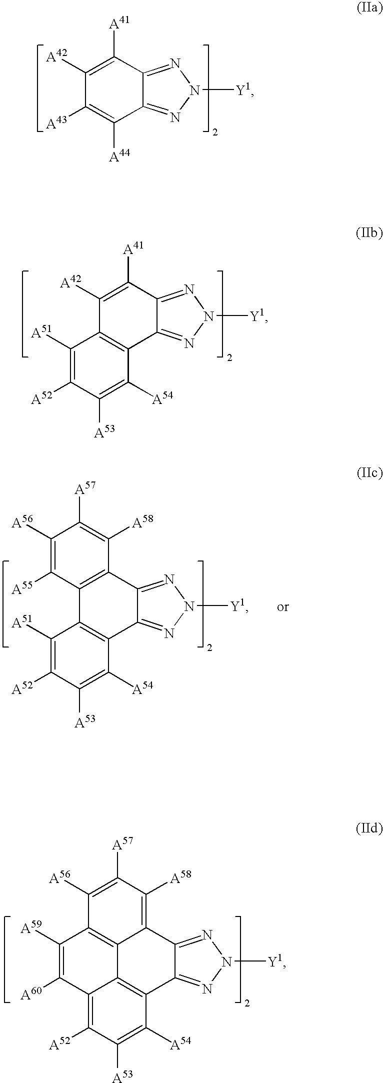
wherein A⁵² and A⁴³ are a group of formula 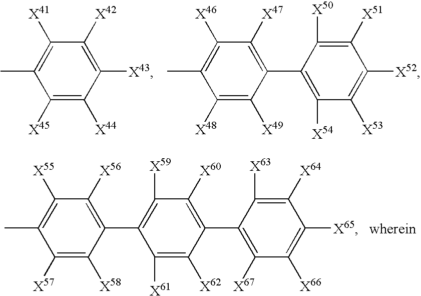
X⁴¹, X⁴², X⁴³, X⁴⁴, X⁴⁵, X⁴⁶, X⁴⁷, X⁴⁸, X⁴⁹, X⁵⁰, X⁵¹, X⁵², X⁵³, X⁵⁴, X⁵⁵, X⁵⁶, X⁵⁷, X⁵⁸, X⁵⁹, X⁶⁰, X⁶¹, X⁶², X⁶³, X⁶⁴, X⁶⁵, X⁶⁵ and X⁶⁷ are independently of each other H, fluorine, CN, C₁-C₂₄alkyl, C₅-C₁₂cycloalkyl, C₇-C₂₅aralkyl, C₁-C₂₄perfluoroalkyl, C₆-C₁₄perfluoroaryl, or C₁-C₂₄haloalkyl, C₆-C₁₀aryl, which can optionally be substituted by one, or more C₁-C₈alkyl, or C₁-C₈alkoxy groups; C₁-C₂₄alkoxy, C₁-C₂₄alkylthio, —NR²⁵R²⁶, —CONR²⁵R²⁶, or —COOR²⁷,
or
two groups X⁴¹, X⁴², X⁴³, X⁴⁴, X⁴⁵, X⁴⁶, X⁴⁷, X⁴⁸, X⁴⁹, X⁵⁰,X⁵¹,X⁵², X⁵³, X⁵⁴, X⁵⁵, X⁵⁶, X⁵⁷, X⁵⁸, X⁵⁹, X⁶⁰, X⁶¹, X⁶², X⁶³, X⁶⁴, X⁶⁵, X⁶⁶ and X⁶⁷, which are neighbouring to each other, are a group 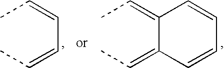
or A⁴³ or A⁵² are a group of formula 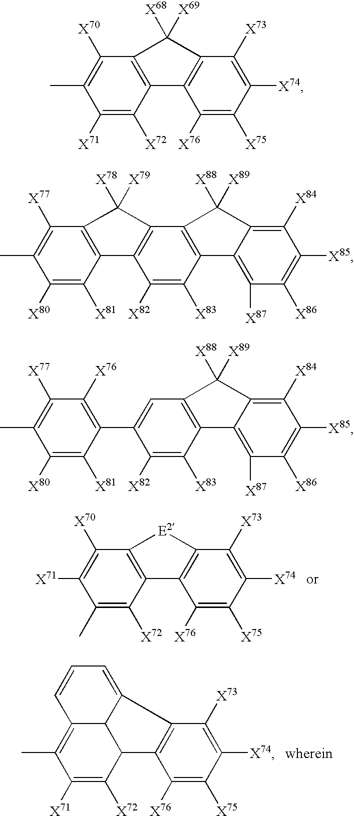
X⁶⁸, X⁶⁹, X⁷⁸, X⁷⁹, X⁸⁸ and X⁸⁹ are independently of each other C₁-C₂₄alkyl, which can be interrupted by one or two oxygen atoms,
X⁷⁰, X⁷¹, X⁷², X⁷³, X⁷⁴, X⁷⁵, X⁷⁶, X⁷⁷, X⁸⁰, X⁸¹, X⁸², X⁸³, X⁸⁴, X⁸⁵, X⁸⁶ and X⁸⁷ are independently of each other H, CN, C₁-C₂₄alkyl, C₆-C₁₀aryl, C₁-C₂₄alkoxy, C₁-C₂₄alkylthio, —NR²⁵R²⁶, —CONR²⁵R²⁶, or —COOR²⁷,
E^{2′} is —S—, —O—, or —NR^{25′}—,
A⁴¹, A⁴² and A⁴⁴ are independently of each other hydrogen, halogen, C₁-C₂₄alkyl, C₁-C₂₄perfluoroalkyl, C₆-C₁₄perfluoroaryl, C₅-C₁₂cycloalkyl, C₇-C₂₅aralkyl, C₁-C₂₄haloalkyl, C₆-C₁₈aryl, —NR²⁵R²⁶, —CONR²⁵R²⁶, or —COOR²⁷, or C₂-C₁₀heteroaryl,
or
A⁵¹, A⁵³, A⁵⁴, A⁵⁵, A⁵⁶, A⁵⁷, A⁵⁸, A⁵⁹ and A⁶⁰ are independently of each other H, fluorine, CN, C₁-C₂₄alkyl, C₁-C₂₄alkoxy, C₁-C₂₄alkylthio, C₅-C₁₂cycloalkyl, C₇-C₂₅aralkyl, C₁-C₂₄perfluoroalkyl, C₆-C₁₄perfluoroaryl, C₁-C₂₄haloalkyl, C₆-C₁₈aryl, —NR²⁵R²⁶, —CONR²⁵R²⁶, or —COOR²⁷, or C₂-C₁₀heteroaryl,
wherein
R²⁵ and R²⁶ are independently of each other H, C₆-C₁₈aryl, C₇-C₁₈aralkyl, or C₁-C₂₄alkyl, or R²⁵ and R²⁶ together form a five or six membered ring,
R²⁷ is C₁-C₂₄alkyl, and
Y¹ is a group of formula 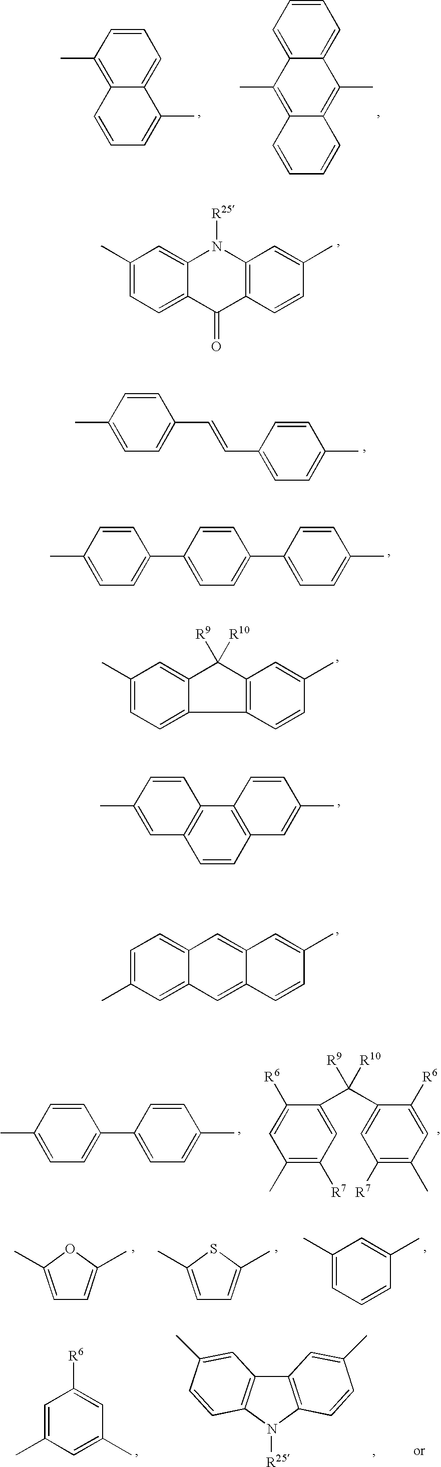 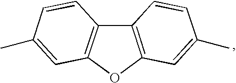
wherein
R⁶ is C₁-C₂₄alkoxy or —O—C₇-C₂₅aralkyl, R⁷ is H, or C₁-C₂₄alkyl, R⁹ and R¹⁰ are independently of each other C₁-C₂₄alkyl, which can be interrupted by one or two oxygen atoms, and
R^{25′} is C₁-C₂₄alkyl or C₆-C₁₀aryl.

8. A 2H-benzotriazole compound according to claim 1, wherein the 2H-benzotriazole is a compound of formula 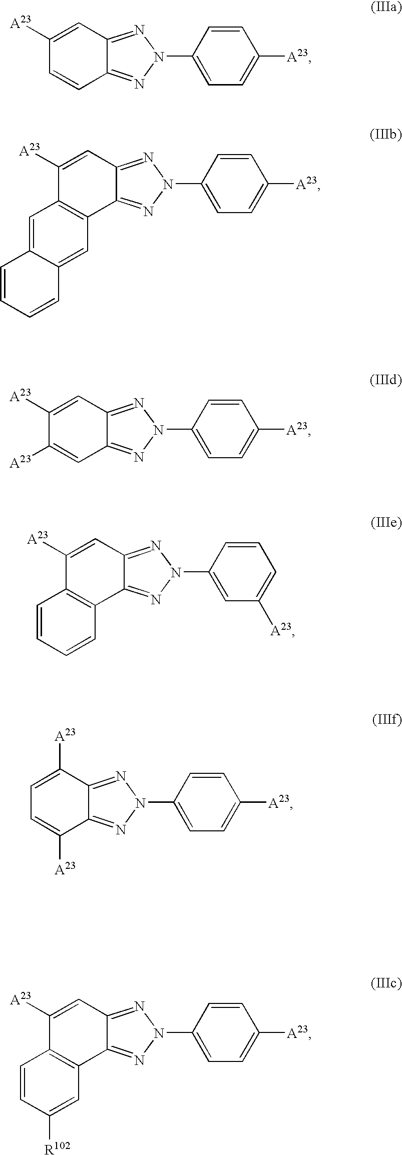
wherein R¹⁰² is C₁-C₂₄alkyl or H,
A²³ is a group of formula 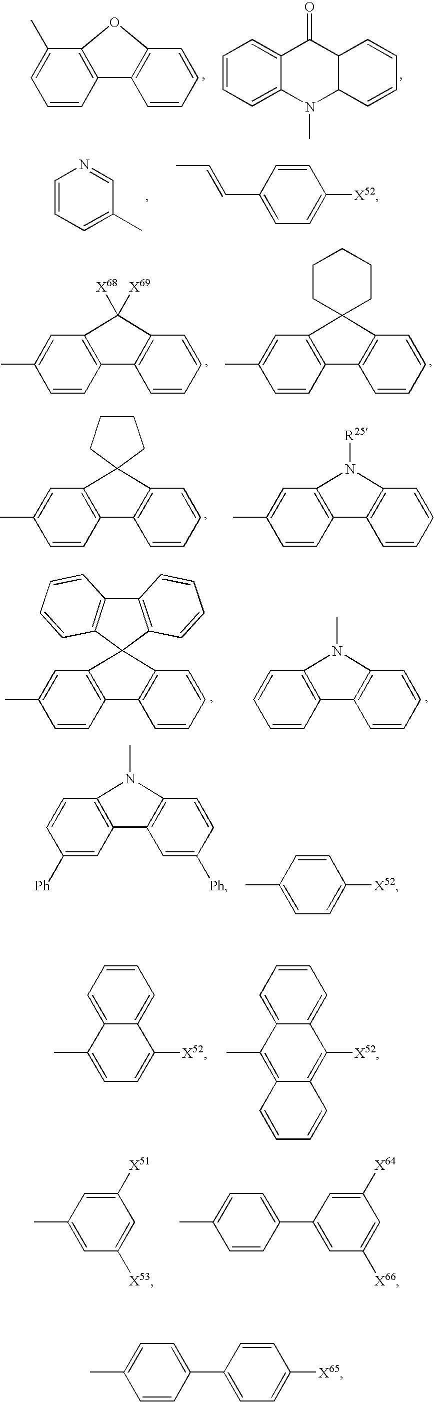 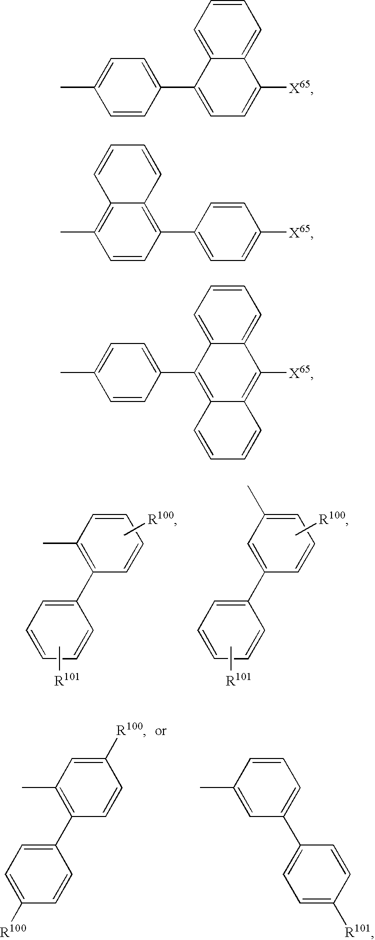
wherein R¹⁰⁰ and R¹⁰¹ are independently of each other H, C₁-C₂₄alkyl, or 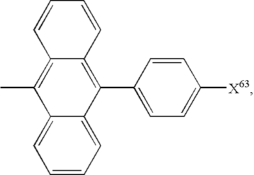
wherein X⁵¹, X⁵², X⁵³, X⁶³, X⁶⁴, X⁶⁵ and X⁶⁶ are independently of each other fluorine, C₁-C₂₄alkyl, C₅-C₁₂cycloalkyl, which can optionally be substituted by one or two C₁-C₈alkyl groups, or 1-adamantyl, C₁-C₂₄perfluoroalkyl, C₆-C₁₄perfluoroaryl, NR²⁵R²⁶, wherein R²⁵ and R²⁶ are C₆-C₁₄aryl, which can be substituted by one or two C₁-C₂₄alkyl groups, or
R²⁵ and R²⁶ together form a five or six membered heterocyclic ring,
or a compound of formula 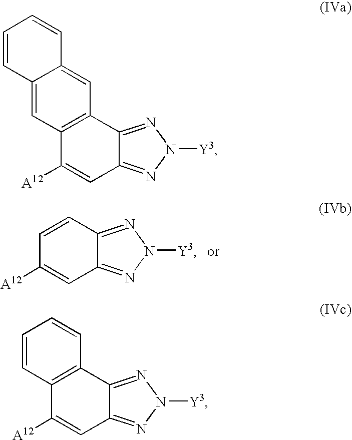
wherein Y³ is as defined above, or is 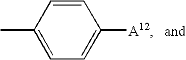
A¹² is NR²⁵R²⁶, 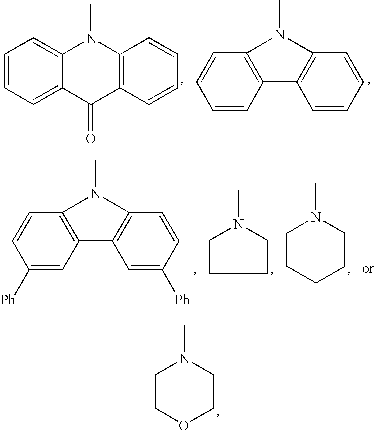
wherein R²⁵ and R²⁶ are C₆-C₁₄aryl, which can optionally be substituted by one or two C₁-C₈alkyl groups or C₁-C₈alkoxy groups, or a compound of formula IVa, IVb, or IVc, wherein A¹² is 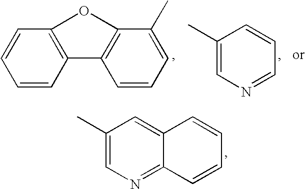
and Y³ is is a compound of formula 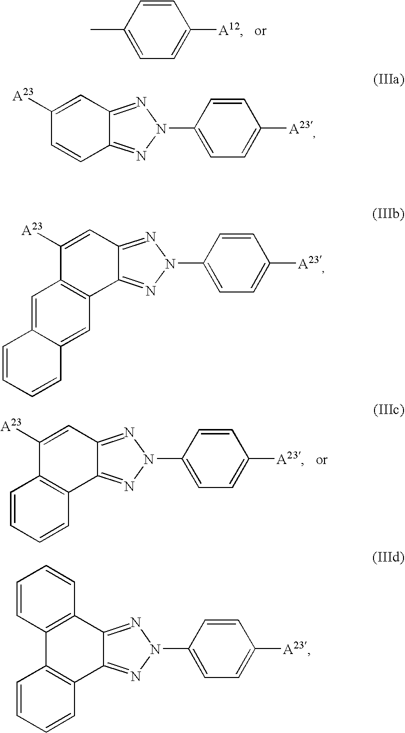
wherein A²³ and A^{23′} are independently of each other a group of formula 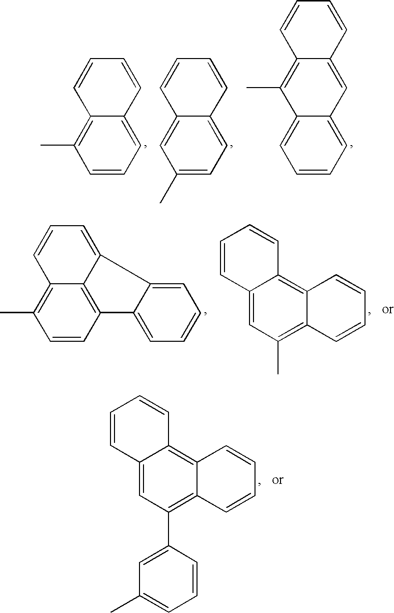
a compound of formula 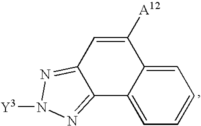
wherein A¹² is H, a group of formula 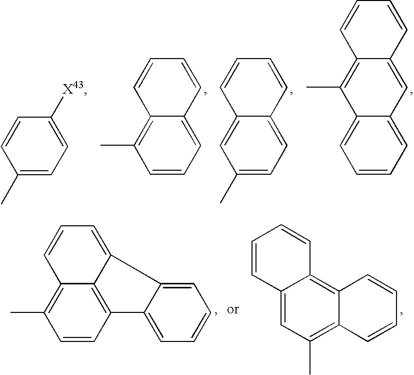
wherein X⁴³ is C₁-C₂₄alkyl, Y³ is a group of formula 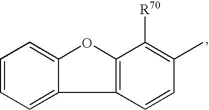
wherein R⁷⁰ is C₁-C₂₄alkyl.

9. A 2H-benzotriazole compound according to claim 8, wherein the 2H-benzotriazole is a compound of formula 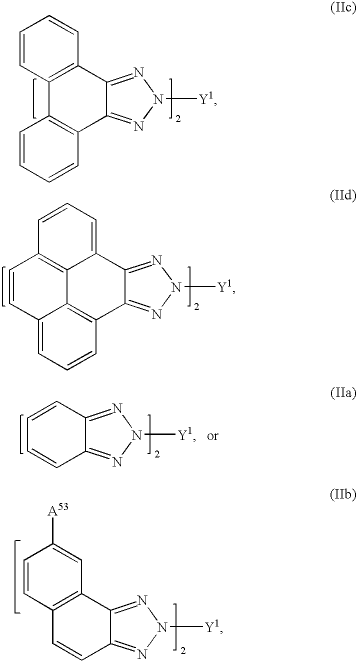
wherein A⁵³ is C₁-C₂₄alkyl, or H,
Y¹ is a group of formula 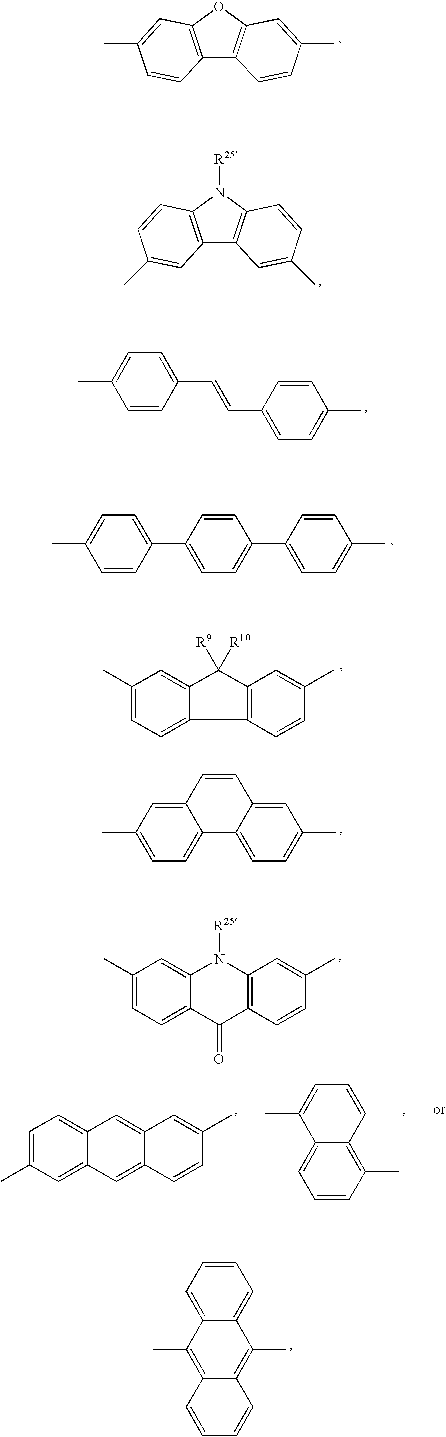
wherein R⁹ and R¹⁰ are independently of each other C₁-C₂₄alkyl which can be interrupted by one or two oxygen atoms, and R^{25′} is C₁-C₂₄alkyl.

10. An electroluminescent device, comprising a 2H-benzotriazole compound according to claim 1.

11. The electroluminescent device according to claim 10, wherein the electroluminescent device comprises in this order
(a) an anode
(b) a hole injecting layer and/or a hole transporting layer
(c) a light-emitting layer
(d) optionally an electron transporting layer and
(e) a cathode.

12. The electroluminescent device according to claim 11, wherein the 2H-benzotriazole compound forms the light-emitting layer.

13. An electrophotographic photoreceptor, photoelectric converter, solar cell, image sensor or dye laser comprising a 2H-benzotriazole compound according to claim 1.

14. A 2H-benzotriazole compound according to claim 1, wherein at least one of the substituents A²¹, A²², A²³, A²⁴, A¹¹, A¹², A¹³, A¹⁴, A¹⁵, A¹⁶, A¹⁷ and A¹⁸ A²¹, A²², A²³, A²⁴, A¹¹, A¹², A¹³, A¹⁴, A¹⁵, A¹⁶, A¹⁷ and A¹⁸ is C₆-C₂₄aryl which is substituted by fluorine, C₁-C₂₄alkyl, C₅-C₁₂cycloalkyl, C₇-C₂₅aralkyl, C₁-C₂₄perfluoroalkyl, C₆-C₁₄perfluoroaryl or C₁-C₂₄haloalkyl; thiophenyl, pyrrolyl, furanyl, benzoxazolyl or benzothiazolyl which is substituted by fluorine, C₁-C₂₄alkyl, C₅-C₁₂cycloalkyl, C₇-C₂₅aralkyl, C₁-C₂₄perfluoroalkyl, C₆-C₁₄perfluoroaryl or C₁-C₂₄haloalkyl,
or a group of formula 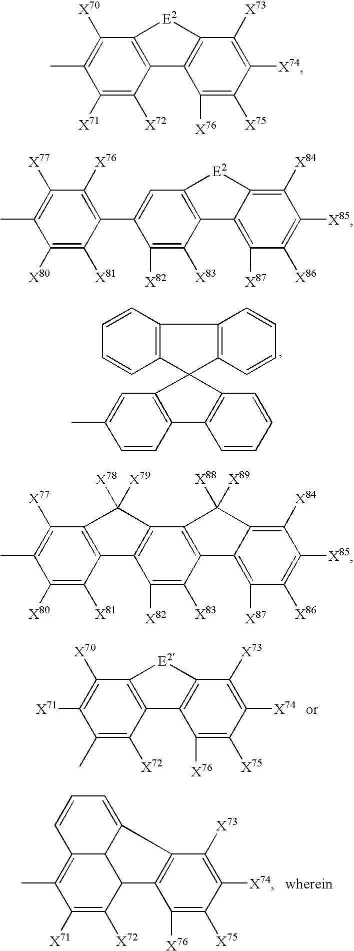
X⁷⁰, X⁷¹, X⁷², X⁷³, X⁷⁴, X⁷⁵, X⁷⁶, X⁷⁷, X⁸⁰, X⁸¹, X⁸², X⁸³, X⁸⁴, X⁸⁵, X⁸⁶, and X⁸⁷ are independently of each other E and/or interrupted by D, C₁-C₂₄perfluoroalkyl, C₆-C₁₄perfluoroaryl, C₅-C₁₂cycloalkyl, C₅-C₁₂cycloalkyl which is substituted by G and/or interrupted by S—, —O— or —NR²⁵—; —NR²⁵R²⁶, C₁-C₂₄alkylthio, —PR³, R³², C₅-C₁₂cycloalkoxy, C₅-C₁₂cycloalkoxy which is substituted by G, C₆-C₂₄aryl, C₆-C₂₄aryl which is substituted by G, C₁-C₂₄alkyl, C₅-C₁₂cycloalkyl, C₇-C₂₅aralkyl, C₁-C₂₄perfluoroalkyl, C₆-C₁₄perfluoroaryl, or C₁-C₂₄haloalkyl; C₂-C₂₀heteroaryl, C₂-C₂₀heteroaryl which is substituted by G, fluorine, C₁-C₂₄alkyl, C₅-C₁₂cycloalkyl, C₇-C₂₅aralkyl, C₁-C₂₄perfluoroalkyl, C₆-C₁₄perfluoroaryl, or C₁-C₂₄haloalkyl; C₂-C₂₄alkenyl, C₂-C₂₄alkynyl, C₁-C₂₄alkoxy, C₁-C₂₄alkoxy which is substituted by E and/or interrupted by D, C₇-C₂₅aralkyl, C₇-C₂₅aralkyl, which is substituted by G, C₇-C₂₅aralkoxy, C₇-C₂₅aralkoxy which is substituted by G, or —CO—R²⁸,
or
two groups X⁷⁰, X⁷¹, X⁷², X⁷³, X⁷⁴, X⁷⁵, X⁷⁶, X⁷⁷, X⁸⁰, X⁸¹, X⁸², X⁸³, X⁸⁴, X⁸⁵, X⁸⁶, and X⁸⁷, which are neighbouring to each other, are a group 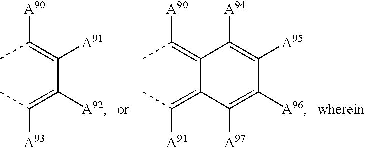
A⁹⁰, A⁹¹, A⁹², A⁹³, A⁹⁴, A⁹⁵, A⁹⁶ and A⁹⁷ are independently of each other H, halogen, hydroxy, C₁-C₂₄alkyl, C₁-C₂₄alkyl which is substituted by E and/or interrupted by D, C₁-C₂₄perfluoroalkyl, C₆-C₁₄perfluoroaryl, C₅-C₁₂cycloalkyl, C₅-C₁₂cycloalkyl which is substituted by G and/or interrupted by S—, —O— or —NR²⁵—; C₅-C₁₂cycloalkoxy, C₅-C₁₂cycloalkoxy which is substituted by G, C₆-C₂₄aryl, C₆-C₂₄aryl which is substituted by G, C₂-C₂₀heteroaryl, C₂-C₂₀heteroaryl which is substituted by G, C₂-C₂₄alkenyl, C₂-C₂₄alkynyl, C₁-C₂₄alkoxy, C₁-C₂₄alkoxy which is substituted by E and/or interrupted by D, C₇-C₂₅aralkyl, C₇-C₂₅aralkyl, which is substituted by G, C₇-C₂₅aralkoxy, C₇-C₂₅aralkoxy which is substituted by G, or —CO—R²⁸,
E² is —CR²³═CR²⁴— or —CX⁶⁸X⁶⁹—,
E^{2′} is —SiR³⁰R³¹—; —POR³²—; —S—, —O—, or —NR^{25′}—, wherein R^{25′} is C₁-C₂₄alkyl, or C₆-C₁₀aryl,
X⁶⁸, X⁶⁹, X⁷⁸, X⁷⁹, X⁸⁸ and X⁸⁹ are independently of each other C₁-C₁₈ alkyl, C₁-C₂₄alkyl which is substituted by E and/or interrupted by D, C₆-C₂₄aryl, C₆-C₂₄aryl which is substituted by G, C₂-C₂₀heteroaryl, C₂-C₂₀heteroaryl which is substituted by G, C₂-C₂₄alkenyl, C₂-C₂₄alkynyl, C₁-C₂₄alkoxy, C₁-C₂₄alkoxy which is substituted by E and/or interrupted by D, or C₇-C₂₅aralkyl, or
X⁷⁸ and X⁷⁹, and/or X⁸⁸ and X⁸⁹ form a ring, or
X⁶⁸ and X⁷⁰, X⁶⁹ and X⁷³, X⁷⁷ and X⁷⁸ and/or X⁸⁴ and X⁸⁹ are a group 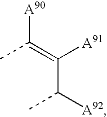
D is —CO—; —COO—; —S—; —SO—; —SO₂—; —O—; —NR²⁵—; —SiR³⁰R³¹; —POR³²—; —CR²³═CR²⁴—; or —C≡C—; and
E is —OR²⁹; SR²⁹; NR²⁵R²⁶; —COR²⁸; —COOR²⁷; —CONR²⁵R²⁶; —CN; —OCOOR²⁷; or halogen;
G is E, or C₁-C₂₄alkyl, wherein
R²³, R²⁴, R²⁵ and R²⁶ are independently of each other H; C₆-C₁₈aryl; C₆-C₁₈aryl which is substituted by C₁-C₂₄alkyl, or C₁-C₂₄alkoxy; C₁-C₂₄alkyl; or C₁-C₂₄alkyl which is interrupted by —O—; or
R²⁵ and R²⁶ together form a five or six membered ring, R²⁷ and R²⁸ are independently of each other H; C₆-C₁₈aryl; C₆-C₁₈aryl which is substituted by C₁-C₂₄alkyl, or C₁-C₂₄alkoxy; C₁-C₂₄alkyl; or C₁-C₂₄alkyl which is interrupted by —O—,
R²⁹ is H; C₆-C₁₈aryl; C₆-C₁₈aryl, which is substituted by C₁-C₂₄alkyl, or C₁-C₂₄alkoxy; C₁-C₂₄alkyl; or C₁-C₂₄alkyl which is interrupted by —O—,
R³⁰ and R³¹ are independently of each other C₁-C₂₄alkyl, C₆-C₁₈aryl, or C₆-C₁₈aryl, which is substituted by C₁-C₂₄alkyl, and
R³² is C₁-C₂₄alkyl, C₆-C₁₈aryl, or C₆-C₁₈aryl which is substituted by C₁-C₂₄alkyl.

15. A 2H-benzotriazole compound according to claim 2, wherein at least one of the substituents X⁴¹, X⁴², X⁴³, X⁴⁴, X⁴⁵, X⁴⁶, X⁴⁷, X⁴⁸, X⁴⁹, X⁵⁰, X⁵¹, X⁵², X⁵³, X⁵⁴, X⁵⁵, X⁵⁶, X⁵⁷, X⁵⁸, X⁵⁹, X⁶⁰, X⁶¹, X⁶², X⁶³, X⁶⁴, X⁶⁵, X⁶⁶ and X⁶⁷ is fluorine, —NR²⁵R²⁶, C₁-C₂₄alkyl, C₅-C₁₂cycloalkyl, C₇-C₂₅aralkyl, C₁-C₂₄perfluoroalkyl, C₆-C₁₄perfluoroaryl or C₁-C₂₄haloalkyl.

16. A 2H-benzotriazole compound to claim 5, wherein Y¹ is a group of formula

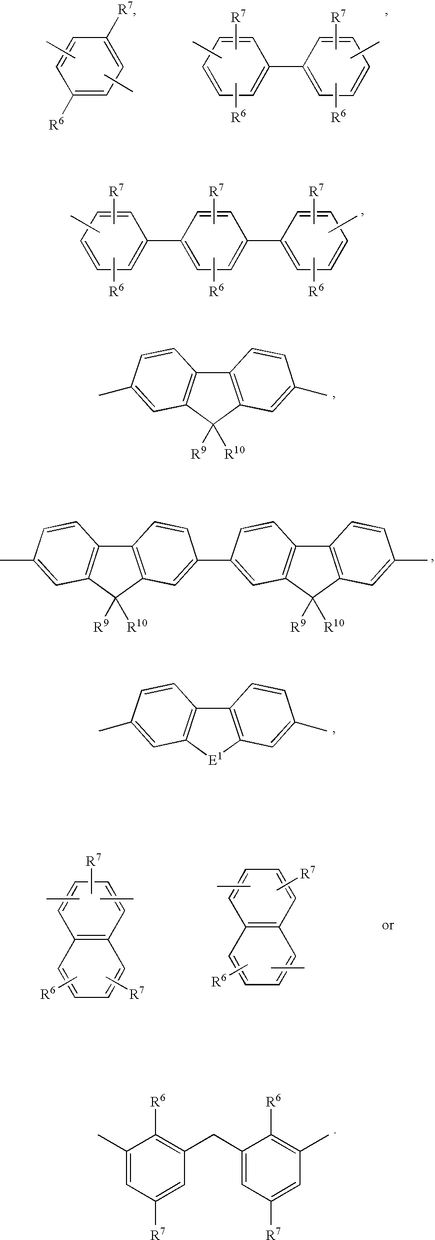

17. A 2H-benzotriazole compound to claim 6, wherein at least one of the substituents X⁴¹, X⁴², X⁴³, X⁴⁴, X⁴⁵, X⁴⁶, X⁴⁷, X⁴⁸, X⁴⁹, X⁵⁰, X⁵¹, X⁵², X⁵³, X⁵⁴, X⁵⁵, X⁵⁶, X⁵⁷, X⁵⁸, X⁵⁹, X⁶⁰, X⁶¹, X⁶², X⁶³, X⁶⁴, X⁶⁵, X⁶⁶ and X⁶⁷ is fluorine, —NR²⁵R²⁶, C₁-C₂₄alkyl, C₅-C₁₂cycloalkyl, C₇-C₂₅aralkyl, C₁-C₂₄perfluoroalkyl, C₆-C₁₄perfluoroaryl, especially pentafluorophenyl, or C₁-C₂₄haloalkyl,
and when A²¹, A²² or A²⁴ is C₂-C₁₀heteroaryl, said C₂-C₁₀heteroaryl is a group of formula

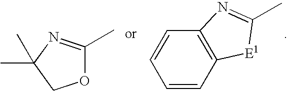

18. A 2H-benzotriazole compound to claim 7, wherein at least one of the substituents X⁴¹, X⁴², X⁴³, X⁴⁴, X⁴⁵, X⁴⁶, X⁴⁷, X⁴⁸, X⁴⁹, X⁵⁰, X⁵¹, X⁵², X⁵³, X⁵⁴, X⁵⁵, X⁵⁶, X⁵⁷, X⁵⁸, X⁵⁹, X⁶⁰, X⁶¹, X⁶², X⁶³, X⁶⁴, X⁶⁵, X⁶⁶ and X⁶⁷ is fluorine, —NR²⁵R²⁶, C₁-C₂₄alkyl, C₅-C₁₂cycloalkyl, C₇-C₂₅aralkyl, C₁-C₂₄perfluoroalkyl, C₆-C₁₄perfluoroaryl, especially pentafluorophenyl, or C₁-C₂₄haloalkyl,
and when A²¹, A²² or A²⁴ is C₂-C₁₀heteroaryl, said C₂-C₁₀heteroaryl is a group of formula [chemical structure]
